(19) United States
(12) Patent Application Publication
(10) Pub. No.: US 2026/0096885 A1
(43) Pub. Date: Apr. 9, 2026

(54) DEVICES AND SYSTEMS FOR REPAIRING A HEART VALVE

(71) Applicant: Evalve, Inc., Santa Clara, CA (US)

(72) Inventors: Jarad Waisblatt, San Jose, CA (US); Michael C. Nasr, Benicia, CA (US); Thomas R. Yssels, Mountain View, CA (US); Luke Thompson, Redwood City, CA (US); Daniel Davis, Campbell, CA (US); Matthew G. Jew, San Francisco, CA (US); Ariel B. Reid, Redwood City, CA (US); Cindy Chou, San Mateo, CA (US); Parisa Pourattar, Newark, CA (US); Grant Glaze, San Francisco, CA (US); Luke R. Mathson, San Francisco, CA (US); Anna E. Wing, Campbell, CA (US); Philip Crompton, San Jose, CA (US); Chenzhe Xu, San Jose, CA (US); Karen J. Wang, Sunnyvale, CA (US); Stephanie A. Kozuma, San Mateo, CA (US); Joanna M. Ignacio, Dublin, CA (US); Jill Mccoy, Los Altos, CA (US)

(73) Assignee: Evalve, Inc., Santa Clara, CA (US)

(21) Appl. No.: 19/228,859

(22) Filed: Jun. 5, 2025

Related U.S. Application Data
(60) Provisional application No. 63/656,690, filed on Jun. 6, 2024.

(51) Int. Cl. A61F 2/24

(52) U.S. Cl. CPC A61F 2/2466; A61F 2/246

(57) ABSTRACT

A delivery system includes a delivery catheter that is releasably coupled to a fixation device and a delivery handle that is coupled to the delivery catheter. A lock control assembly of the delivery handle has a releasable lock line handle operable to lock and unlock components of the fixation device. The lock line handle has a shaft body and a lever arm that respectively define first and second openings. A lock line extending from the fixation device and through the delivery catheter has a first end portion fixedly coupled to the lock line handle and a second end portion that extends through the first and second openings. The lock line is free to move through the openings when the lever arm is in a first position and is secured when the lever arm is in a second position.

### CROSS-REFERENCE TO RELATED APPLICATIONS

This application claims the benefit of the filing date of U.S. Provisional Application No. 63/656,690, filed Jun. 6, 2024, the disclosure of which is hereby incorporated by reference.

### BACKGROUND

The cardiac cycle is divided into two phases—diastole and systole. Diastole is generally characterized by the muscular relaxation of the heart and the filling of its chambers with blood. On the other hand, systole is generally characterized by the muscular contraction of the ventricles which pumps blood from the ventricles to the arteries. During ventricular systole, ventricular pressure increases relative to atrial pressure resulting in the closure of the mitral valve and the tricuspid valve. The mitral valve separates the left atrium from the left ventricle, and the tricuspid valve separates the right atrium from the right ventricle. These valves operate as check valves preventing blood from flowing back into the atria during ventricular contraction. However, valvular insufficiency may appear in one or both of these valves which may result in a regurgitative flow back into the atrium across the effected valve. Such regurgitative flow can be in the form of mitral valve regurgitation (“MVR”) and/or tricuspid valve regurgitation (“TVR”). Left untreated, MVR and TVR can lead to severe health consequences, such as progressive heart failure, cardiac arrhythmias, pulmonary hypertension, stroke, and endocarditis, to name a few.

MVR and TVR can have a variety of etiologies which typically fall into the categories of degenerative (primary) and functional (secondary) regurgitation. Degenerative valve regurgitation principally occurs due to abnormalities or degeneration of the valve apparatus, such as the valve leaflets, valve annulus, chordae tendineae, and/or papillary muscles. One example of a degenerative valve condition is mitral valve prolapse. Functional valve regurgitation is often a secondary condition that arises from underlying heart conditions or diseases that affect the structure or function of the heart. Examples of conditions that can result in functional regurgitation include dilated cardiomyopathy, ischemic heart disease, pulmonary hypertension, and heart failure. Regardless of the underlying condition precipitating the regurgitative flow, the primary mechanism by which regurgitation occurs is the failure of the valve leaflets to properly and completely seal or coapt during systole which allows a jet of blood to flow back into the atrium between the effected leaflets.

Treatment options for MVR and TVR generally include Guideline-Directed Medical Therapy (“GDMT”), valve replacement, and valve repair. GDMT usually involves the administration of a combination of drugs that treat an underlying heart condition. Valve replacement and repair may include open-heart surgical options and catheter-based options. Catheter-based repair procedures are sometimes referred to as transcatheter edge-to-edge repair (“TEER”).

### BRIEF SUMMARY OF THE DISCLOSURE

In a first aspect of the present disclosure, a delivery system for delivering a fixation device to a heart valve may include a delivery catheter and a delivery handle. The delivery catheter may have a proximal end portion and a distal end portion and may be configured to releasably couple to the fixation device. The delivery handle may be coupled to the proximal end portion of the delivery catheter and may have a lock control assembly. The lock control assembly may have a lock line handle that may be releasably coupled to the delivery handle and actuatable between a locked position and an unlocked position. The lock line handle may have a handle shaft. The handle shaft may have a shaft body that may define a first opening and a lever arm that may define a second opening. The lever arm may be connected to the shaft body and may be rotatable relative to the shaft body between a first position in which the first and second openings are axially aligned and a second position in which the first and second openings are angled relative to each other. The delivery system may also include a lock line for locking and unlocking the fixation device. The lock line may have a first end portion that may be fixedly coupled to the lock line handle and a second end portion that may extend into the first and second openings of the lock line handle. The second end portion of the lock line may be free to travel through the first and second openings when the lever arm is in the first position, and when the lever arm is in the second position, the second end portion of the lock line may be secured to the handle shaft such that it is prevented from moving through the first and second openings.

Additionally, the lever arm may include a lever body and a post that may extend from the lever body. The post may define the second opening through which the lock line extends, and the shaft body may define a post opening that may be configured to receive the post of the lever arm. The first opening may intersect the post opening. Further, the lever body may include a third opening. The third opening may be axially aligned with the second opening such that the second end portion of the lock line extends into the first, second, and third openings. Alternatively, the shaft body may include a post extending from the shaft body. The post may define the first opening through which the lock line extends, and the lever arm may define a post opening configured to receive the post of the shaft body and the second opening may intersect the post opening.

Also, the lock line handle may include a lock knob, and the handle shaft may extend from the lock knob. The lock knob may have an opening that may extend therethrough. The handle shaft may extend through the opening of the lock knob such that the lever arm is positioned at one side of the lock knob and the shaft body is at least partially positioned at another side of the lock knob. The lock knob may have a pear shape, and the lock line handle may include a cylindrical body that may extend from the lock knob and at least partially about the handle shaft. The cylindrical body may have a plurality of detents that may extend into an exterior thereof. The lock control assembly may include a locking system that may have a lock body coupled to the delivery handle. The lock body may have a pin configured to independently engage the detents in the cylindrical body. The detents may have a first detent representing an unlocked position in which the lock line is tensioned to lock the fixation device, and a second detent representing a locked position in which tension on the lock line is released to lock the fixation device. The cylindrical body may include a circumferential channel intersecting the plurality of detents. The pin may be configured to engage the circumferential channel when not engaged with any of the detents, and the lock body may be moveable between a locked position in which the pin engages one of the channel and the detents and an unlocked position in which the pin is disengaged from the channel and the detents which permits the lock line handle from being released from the delivery handle.

Further, the lock control assembly may include a lock handle housing. The lock handle housing may have a lock handle extension that may be configured to receive the cylindrical body of the lock knob assembly, and the lock body may be coupled to the lock handle extension. The locking system may also include a biasing member positioned between the lock body and the lock handle extension. The biasing member may urge the lock body toward the locked position thereof. The lock handle housing may include a fluid volume and an inlet/outlet port in fluid communication with the fluid volume. The lock control assembly may further include a spool. The spool may be received within the fluid volume of the lock handle housing and may have a spool track along which the first end portion of the lock line extends. The spool may be releasably coupled to the lock handle shaft and may be configured to rotate with the lock line handle such that rotation in a second direction releases tension on the lock line.

Additionally, the spool may include a first lock line opening and a second lock line opening. The first end portion of the lock line may extend through the first lock line opening of the spool and into the first opening of the shaft body of the lock handle shaft where the first end portion of the lock line may be fixedly secured. The second end portion of the lock line may extend through the second lock line opening in the spool, the first opening in the shaft body, and the second opening in the lever arm. The delivery handle may also include an actuator rod for moving the fixation device between a plurality of positions. The actuator rod may through the lock handle housing and into the delivery catheter.

In another aspect of the present disclosure, an interventional system for repairing a heart valve may include a multi-catheter system that may comprise a plurality of catheters, a plurality of catheter handles coupled to respective ones of the plurality of catheters, a first attachment assembly, and a second attachment assembly. The first attachment assembly may have a support body coupled to one of the catheter handles and first and second engagement members connected to the support body. At least one of the first and second members may be moveable relative to the support body such that the first and second engagement members may have a first configuration and a second configuration. The first and second engagement members may be further apart in the first configuration than in the second configuration. The interventional system may also include a stabilizer for supporting the multi-catheter system. The stabilizer may have a base, a first attachment coupled to the base, and a second attachment coupled to the base. The first attachment may be configured to attach to the first attachment assembly and may have a first rail and a second rail. The first and second rails at least partially define a slot disposed therebetween. The second attachment may be configured to attach to the second attachment. In this regard, the first and second engagement members may be receivable within the slot while in the second configuration and engage the rails when in the second configuration so as to retain the first attachment assembly within the slot.

Additionally, the first attachment assembly may be slidable along the first and second rails when the first and second engagement members are disposed within the slot and in the first configuration. The first attachment assembly may include a biasing member. The biasing member may urge the first and second engagement members toward the first configuration. The first and second engagement members may each include a capture member. The capture member of the first engagement member may have a shoulder at least partially defining a first channel, and the capture member of the second engagement member may have a shoulder at least partially defining a second channel. The first and second channels may be configured to respectively receive the first and second rails when the first and second engagement members are in the first configuration. Each capture member may include a sloping surface that slopes outwardly in a direction extending from a free end of the capture member toward the shoulder.

Also, the support body may include a first end wall and a second end wall. Each of the end walls may define a first width and may be receivable within the slot. The first and second engagement members may be disposed between the end walls and may collectively define a second width when in the first configuration and a third width when in the second configuration. The second width may be greater than the first width, and the third width may be equal to or less than the first width. The support frame may further include a boss, a central member, and a transverse member. The boss may have a first opening that may be configured to receive one of the catheters of the multi-catheter system. The central member may extend downwardly from the boss and may be positioned between the first and second engagement members. The transverse member may extend from the central member in a direction transverse to the central member and may, at least partially, define the first and second channels. The transverse member may be a plate that may have a planar surface. The rails of the first attachment may have a corresponding planar surface upon which the transverse member may sit when the first attachment assembly is received within the slot.

Further, the first and second members may be pivotably connected via a hinge. The hinge be may at least partially disposed within the support body. The support body may include a mount member connected to one of the catheter handles and a boss extending from the mount member. The boss may at least partially house the hinge. The multi-catheter system may include a first catheter, a first handle coupled to proximal end of the first catheter, and a brake shaft extending from the first handle. The first catheter may be slidably disposed within the brake shaft, and the delivery handle may have a fastening assembly. The fastening assembly may have a static member and a dynamic member. The dynamic member may be moveable relative to the dynamic member in first and second directions such that moving the dynamic member in the first direction arrests movement of the first handle relative to the brake shaft, and moving the dynamic member in the second direction permits first handle to slide along the brake shaft. The support body may include a boss that may include a shaft opening configured to receive the brake shaft.

Continuing with this aspect, the multi-catheter system may include a second catheter and a second catheter handle coupled to the second catheter. The brake shaft may be coupled to the second catheter handle, and the first catheter may extend through the second catheter handle and into the second catheter. The multi-catheter system may also include a third catheter and a third catheter handle coupled to the second catheter. The second catheter may extend through the third catheter handle and into the third catheter. The second attachment assembly may be connected to the third catheter handle. The second and third catheter handles may include at least one actuator configured to bend the second and third catheters, respectively, upon actuation of the actuator. The first catheter may be configured to releasably couple to an implantable device, and the first catheter handle may include a plurality of controls for controlling components of the implantable device.

Additionally, the second attachment assembly may include a first engagement member and a second engagement member. The second attachment of the stabilizer may include a first hook that may be configured to capture the first engagement member of the second attachment assembly and a second hook configured to capture the second engagement member of the second attachment assembly. The first engagement member of the second attachment assembly may be a dynamic pin, and the second engagement member of the attachment assembly may be a static pin. The dynamic pin may be moveable relative to the static pin between a first position and a second position and may be further from the static pin when in the first position than in the second position. The second attachment assembly may include a biasing member engaged to the dynamic pin. The biasing member may urge the dynamic pin towards a first position. Also, the first hook may include a cam surface configured to urge the dynamic pin toward the second position when the static pin is captured by the second hook.

Also, the second attachment assembly may include a bushing, a housing disposed about the bushing and rotatable relative to the bushing, and an O-ring positioned between the bushing and the housing. The O-ring may be in direct contact with the bushing and the housing to resist relative rotation therewith. The bushing may be configured to receive at least one of the plurality of catheters. The first and second engagement members of the second attachment assembly may be coupled to the housing. The first attachment may include a first end wall and a second end wall each at least partially defining the slot. The slot may have a slot length extending from the first end wall to the second end wall. The multi-catheter system may include a first catheter that may have a distal end and a second catheter that may have a distal end. The first catheter may be slidably received within the second catheter such that the distal end of the first catheter may be extendable from the distal end of the second catheter a maximum distance therefrom that is equal to the slot length when the first attachment assembly is coupled to the first attachment.

In a further aspect of the present disclosure, an interventional system for repairing a heart valve may include a multi-catheter system. The multi-catheter system may include a plurality of catheters, a plurality of catheter handles coupled to respective ones of the plurality of catheters, a first attachment assembly, and a second attachment assembly. The second attachment assembly may include a support body coupled to one of the catheter handles and first and second engagement members that may be connected to the support body. The first engagement member may be moveable relative to the second engagement member between a first position and a second position. The interventional system may also include a stabilizer for supporting the multi-catheter system which may have a base, a first attachment coupled to the base, and a second attachment coupled to the base. The first attachment may be configured to couple to the first attachment assembly, and the second attachment may have a first capture member that may define a first recess configured to receive the first engagement member and a second capture member that may define a second recess configured to receive the second engagement member. When the second engagement member is received within the second recess, moving the first engagement member from the first position to the second position allows it to be received within the first recess.

Additionally, the first engagement member of the second attachment assembly may be a dynamic pin, and the second engagement member of the attachment assembly may be a static pin. The first capture member may be a first hook that may extend in a distal direction, and the second capture member may be a second hook that may extend in a proximal direction. The second attachment assembly may include a biasing member that may be engaged to the dynamic pin. The biasing member may urge the dynamic pin towards the first position. The first hook may include a cam surface configured to urge the dynamic pin toward the second position when the static pin is captured by the second hook. The dynamic pin may move back toward the first position when received within the first recess. The second attachment assembly may include a bushing, a housing disposed about the bushing and rotatable relative to the bushing, and an O-ring positioned between the bushing and the housing. The O-ring may be in direct contact with the bushing and the housing to resist relative rotation therewith. The bushing may be configured to receive at least one of the plurality of catheters. Also, the first and second engagement members may be coupled to the housing.

### BRIEF DESCRIPTION OF THE DRAWINGS

FIG. 1A is a cross-sectional representation of a heart illustrating its four valves.

FIG. 1B is a cross-sectional representation of a heart illustrating the left ventricle and left atrium during systole.

FIG. 2A is a schematic view of a mitral valve during normal coaptation.

FIG. 2B is a schematic view of a mitral valve during regurgitate coaptation.

FIGS. 3A and 3B are schematic views of a fixation device according to an embodiment of the present disclosure grasping leaflets of a mitral valve.

FIG. 4A is a perspective view of a fixation device according to another embodiment of the present disclosure.

FIG. 4B is a perspective view of the fixation device of FIG. 4A including a covering.

FIG. 5A is a perspective view of a gripping device of the of the fixation device of FIG. 4A according to an embodiment of the present disclosure.

FIG. 5B is an elevational view of the gripping device of FIG. 5A.

FIG. 6A is a perspective view of a gripping device according to another embodiment of the present disclosure.

FIG. 6B is a partial schematic view of the gripping device of FIG. 6A coupled to a distal element of the fixation device of FIG. 4A.

FIG. 6C is a partial schematic view of a gripping device according to an alternative embodiment of the present disclosure coupled to a distal element according to an alternative embodiment of the present disclosure.

FIG. 7A is an elevational view of a coupling system according to an embodiment of the present disclosure for coupling the fixation device of FIG. 4A and a delivery system.

FIGS. 7B and 7C are schematic views of the coupling system of FIG. 7A in respective first and second configurations.

FIGS. 8A and 8B are schematic cross-sectional views of a coupling system according to another embodiment of the present disclosure for coupling a fixation device, such as the fixation device of FIG. 4A, and a delivery system.

FIGS. 9A-9B, 10A-10B, 11A-11B, 12A-12B and 13A-13C illustrate the fixation device of FIG. 4A in various possible positions during introduction and placement of the device within a mammalian body to perform a therapeutic procedure.

FIG. 14 is a perspective view of the fixation device of FIG. 4A including a locking mechanism according to an embodiment of the present disclosure and illustrating a plurality of proximal element lines and a lock line coupled to the fixation device.

FIG. 15 is an elevational view of the locking mechanism and proximal elements of the fixation device of FIG. 14 and illustrating a lock line and single proximal element line respectively coupled thereto.

FIG. 16 is a schematic view of the fixation device of FIG. 4A coupled to a delivery system and illustrating a plurality of proximal element lines coupled to a shaft of the delivery system.

FIGS. 17A and 17B are partial enlarged views of a distal end portion of the delivery system shaft of FIG. 16 according to an embodiment of the present disclosure.

FIG. 17C is a cross-sectional view of the delivery system shaft taken along line C-C of FIG. 17B.

FIG. 17D is a partial perspective view of a distal end portion of one of the proximal element lines of FIG. 16 including a catch element according to an embodiment of the present disclosure.

FIG. 17E is a partial elevational view of the delivery system shaft of FIG. 17A having holes configured to receive the catch element of FIG. 17D.

FIG. 17F is a partial elevational view of the delivery system shaft of FIG. 17A and an actuator rod disposed therein intersecting the holes of the delivery system shaft.

FIG. 17G is a partial elevational view of a distal end portion of one of the proximal element lines of FIG. 16 including a catch element according to another embodiment of the present disclosure.

FIG. 18A is an enlarged cross-sectional view of the locking mechanism of FIG. 14 taken along a midline thereof and in an unlocked configuration.

FIG. 18B is an enlarged elevational view of the locking mechanism of FIG. 14 and in a locked configuration.

FIG. 18C is a perspective view of a release harness of the locking mechanism of FIG. 14.

FIG. 19A is an elevational view of a locking mechanism of the fixation device of FIG. 4A according to another embodiment of the present disclosure.

FIG. 19B is a transparent perspective view of a binding plate of the locking mechanism of FIG. 19A.

FIG. 19C is an enlarged elevational view of the locking mechanism of FIG. 19A.

FIG. 20 is a partial perspective view of an interventional system according to an embodiment of the present disclosure and including an exemplary delivery system, an exemplary steerable guide system, and an exemplary stabilizer.

FIG. 21A is a partial perspective view of the delivery system of FIG. 20.

FIG. 21B is a partial cutaway view of the delivery system of FIG. 20 and the fixation device of FIG. 4A coupled thereto.

FIG. 22A is a cross-sectional view of a positioner assembly of the delivery system of FIG. 20.

FIG. 22B is a partial perspective view of the positioner assembly of FIG. 22A including a rotation control system according to an embodiment of the present disclosure and in an example of a first configuration.

FIGS. 22C and 22D are partial perspective views of the positioner assembly and rotation control system of FIG. 22B with the rotation control system in an example of a second configuration.

FIG. 23A is a partial perspective view of a gripper control assembly and a fluid management assembly of the delivery system of FIG. 20 and each according to an embodiment of the present disclosure.

FIG. 23B is a partial elevational view of the gripper control assembly of FIG. 23A.

FIG. 23C is a partial cross-sectional view of the gripper control assembly of FIG. 23A taken along a midline thereof.

FIG. 24A is a partial transparent perspective view of a lock control assembly of the delivery system of FIG. 20 according to an embodiment of the present disclosure.

FIG. 24B is another partial transparent perspective view of the lock control assembly of FIG. 24A.

FIG. 24C is an exploded view of the lock control assembly of FIG. 24A.

FIGS. 24D and 24E are perspective views of a lock handle shaft of the lock control assembly of FIG. 24A in an example of an unlocked configuration.

FIGS. 24F and 24G are perspective views of a lock handle shaft of the lock control assembly of FIG. 24A in an example of an locked configuration.

FIG. 24H is a perspective view of a post of lever of lock handle shaft of the lock control assembly of FIG. 24A.

FIG. 24I is a partial transparent perspective view of the lock control assembly of FIG. 24A with a lock knob thereof in an example of a lock configuration.

FIG. 24J is a partial transparent perspective view of the lock control assembly of FIG. 24A with a lock knob thereof in an example of an unlocked configuration.

FIG. 24K is a partial transparent perspective view of the lock control assembly of FIG. 24A with a lock knob thereof in an example of a third configuration.

FIGS. 24L and 24M are partial transparent perspective views of the lock control assembly of FIG. 24A with a release slider thereof in an example of a release position.

FIG. 24N is a partial transparent perspective view of the lock control assembly of FIG. 24A including lock knob thereof being removed.

FIG. 24O is a perspective view of the lock control assembly of FIG. 24A partially disassembled.

FIG. 25A is a cross-sectional view of a delivery catheter fastening assembly of the delivery system of FIG. 20 according to an embodiment of the present disclosure.

FIG. 25B illustrates the delivery catheter fastening system of FIG. 25A being rotated in first and second directions.

FIGS. 25C and 25D illustrate the delivery system of FIG. 20 translating between first and second positions along a shaft of the delivery catheter fastening assembly of FIG. 25A.

FIG. 26 is a partial elevational view of the interventional system of FIG. 20 including a proximal attachment assembly thereof.

FIG. 27A is a perspective view of the proximal attachment assembly of FIG. 26.

FIGS. 27B and 7C are elevational views of the proximal attachment assembly of FIG. 27A in an example of a first configuration and an example of a second configuration, respectively.

FIGS. 27C and 27D are elevational views of the proximal attachment assembly of FIG. 27A in the first configuration and the second configuration, respectively.

FIG. 28 is partial perspective view of the interventional system of FIG. 20 including an exemplary distal attachment assembly thereof.

FIG. 29A illustrates the distal attachment connected to an outer guide catheter handle of the interventional system shown in cutaway.

FIG. 29B is a partial cross-sectional view of the distal attachment assembly of FIG. 29A taken along a midline thereof.

FIG. 30A is a perspective view of the stabilizer of FIG. 20.

FIG. 30B is a partial perspective view of a distal attachment of the stabilizer of FIG. 30A.

FIG. 30C is a partial perspective view of a proximal attachment of the stabilizer of FIG. 30A.

FIGS. 31A and 31B illustrate the distal attachment assembly of FIG. 29A being attached to the distal attachment FIG. 30A.

FIGS. 32A and 32B illustrate the proximal attachment assembly of FIG. 27A attached to the proximal attachment of FIG. 30A.

FIGS. 33A and 33B illustrate the proximal attachment assembly of FIG. 27A being moved relative to the proximal attachment of the stabilizer of FIG. 30A.

FIGS. 34A and 34B illustrate an example of manipulation of the fixation device during a procedure using the interventional system and stabilizer of FIG. 20.

### DETAILED DESCRIPTION

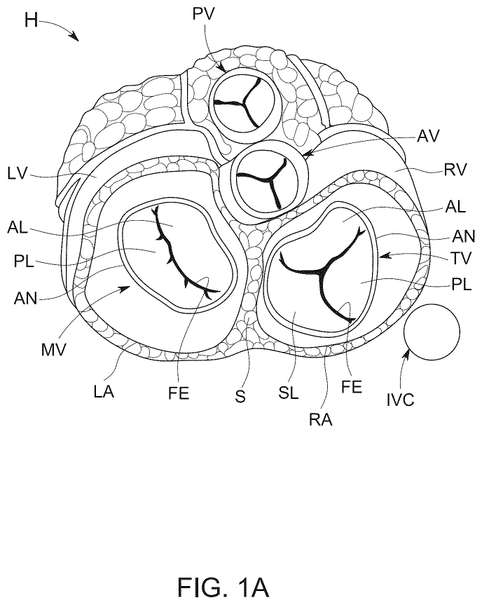

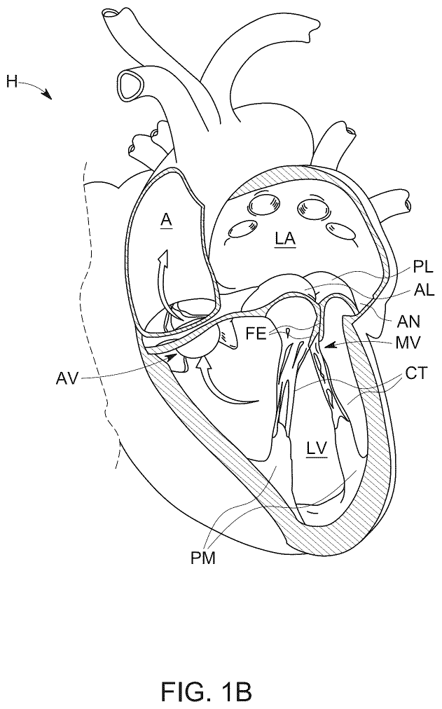

The valves of a normal heart H are illustrated in FIGS. 1A and 1B. These valves include the mitral valve MV, the tricuspid valve TV, the aortic valve AV, and the pulmonary valve PV. The mitral valve MV separates the left atrium LA and the left ventricle LV, and the tricuspid valve TV separates the right atrium RA and the right ventricle RV. The mitral valve MV and the tricuspid valve TV are sometimes referred to as the atrioventricular valves. The mitral valve MV is a bicuspid valve in that it has two leaflets referred to as the posterior leaflet PL and the anterior leaflet AL. The tricuspid valve TV, as the name suggests, has three leaflets referred to as the anterior leaflet AL, the posterior leaflet PL, and the septal leaflet SL.

As illustrated in FIG. 1B, the anterior leaflet AL and posterior leaflet PL of the mitral valve MV extend from a valve annulus AN to respective free edges FE. The free edges FE are secured to the lower portions of the left ventricle LV through chordae tendineae CT (referred to hereinafter as the chordae). The chordae CT include a plurality of branching tendons that are attached to papillary muscles PM at the lower portions of the left ventricle LV and extend upwardly to the lower surfaces of each of the valve leaflets where they are attached. The three leaflets of the tricuspid valve TV similarly extend from a valve annulus AN to respective free edges FE which are secured via chordae to the papillary muscles of the right ventricle RV.

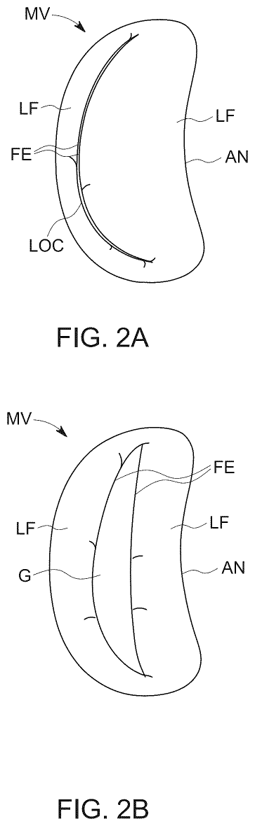

The mitral valve MV depicted in FIGS. 1B and 2A illustrate the proper functioning of an atrioventricular valve during ventricular systole. As the ventricles contract, the free edges FE of adjacent leaflets LF meet along a line of coaptation LOC. The joinder of the leaflets LF at this line of coaptation LOC seals off the ventricle from the atrium and prevents the back flow of blood or “regurgitation” from entering into the atrium. Thus, with the right atrium RA and left atrium LA respectively sealed off by the mitral valve MV and tricuspid valve TV, blood in the left ventricle LV can only flow through the aortic valve AV to the body, and blood in the right ventricle RV can only flow through the pulmonary valve PV to the lungs.

A number of structural defects in the heart H can cause mitral valve regurgitation (“MVR”) and/or tricuspid valve regurgitation (“TVR”). MVR and TVR occur when their respective leaflets LF do not close properly allowing leakage from the ventricle into the atrium. The mitral valve MV depicted in FIG. 2B illustrates valvular insufficiency of an atrioventricular valve resulting in regurgitation. In the depicted example, an enlargement of the heart H may cause the valve annulus AN to become enlarged, making it impossible for the free edges FE of the valve leaflets LF to meet during systole. This may result in a gap G between the leaflets LF which allows blood to leak through the valve. In another example, ruptured or elongated chordae CT can cause a valve leaflet LF to prolapse at least due to inadequate tension transmitted to the leaflet via the chordae CT. While an adjacent leaflet LF may maintain a normal profile, the prolapsing leaflet LF may flail about preventing the proper joinder between the leaflets LF resulting in leakage into the atrium. In a further example, regurgitation can occur in patients who have suffered ischemic heart disease which may result in weak ventricular contractions insufficient to effect proper closure.

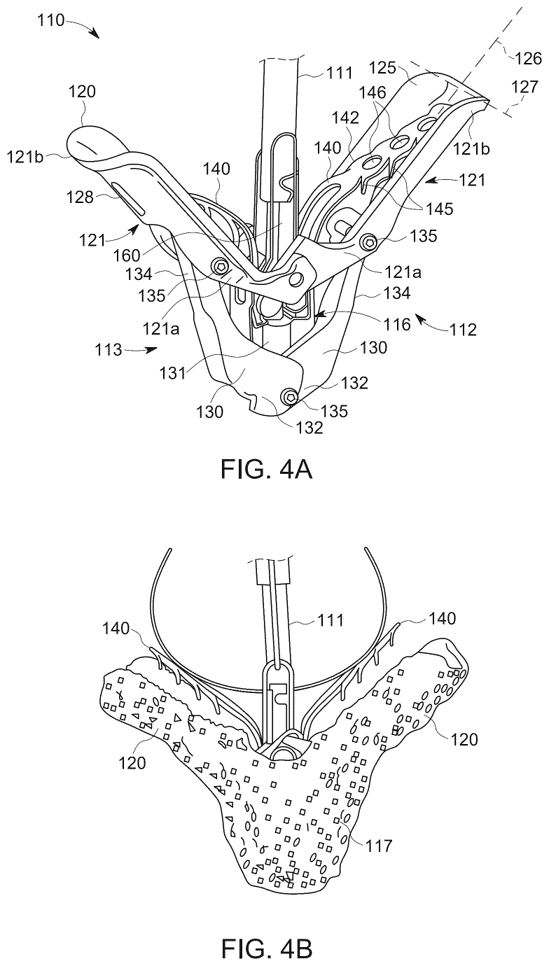

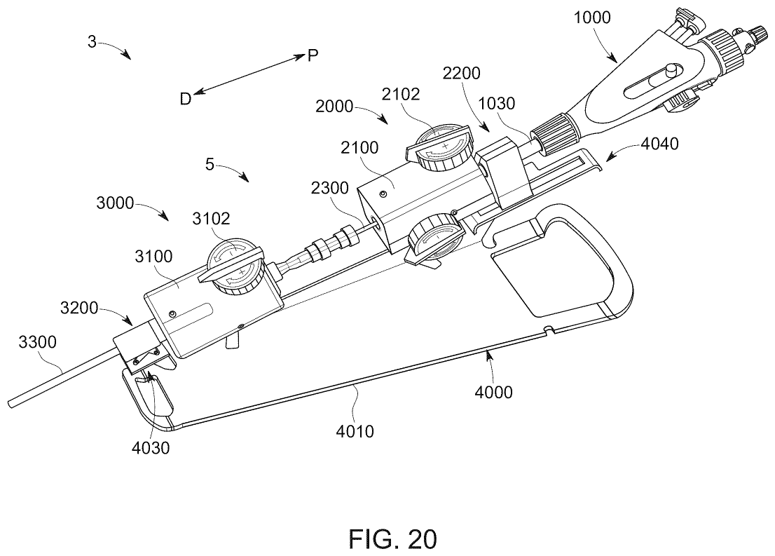

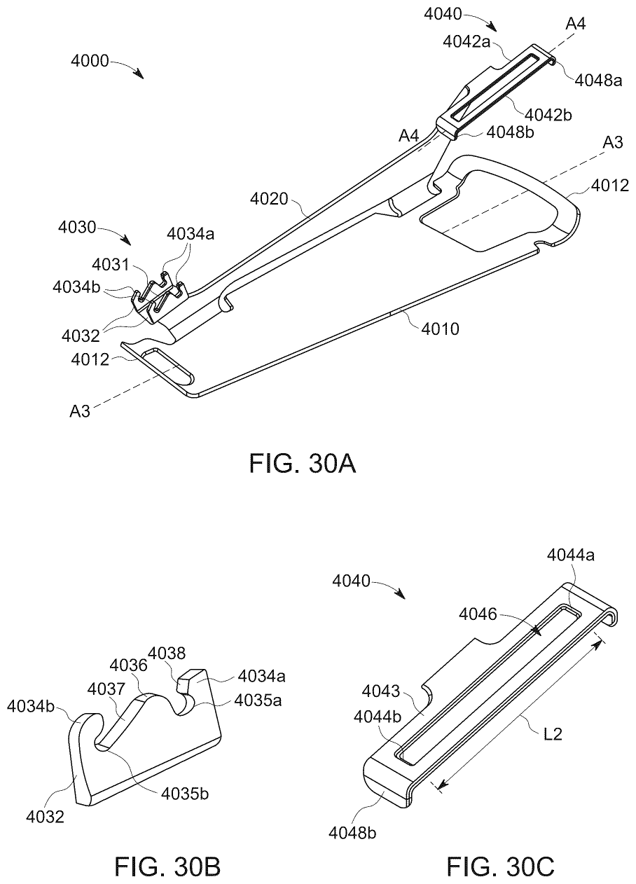

The present disclosure describes exemplary systems, devices, and methods for percutaneously repairing a valve to treat cardiac valve regurgitation, particularly MVR and TVR. For example, an interventional system 3, according to an embodiment of the present disclosure, may include a fixation device 112 (see e.g., FIG. 4A), a delivery system 1000 (see e.g., FIG. 20) for delivery to and deployment of a fixation device (e.g., fixation device 112) within a heart valve, a steerable guide system 5 (see e.g., FIG. 20) for providing a conduit to guide delivery system 1000 and fixation device 112 to the heart valve, and a stabilizer 4000 (see e.g., FIG. 30A) for stabilizing and supporting the use of delivery system 1000 and the steerable guide system 5. The delivery system 1000 and steerable guide system 5 are collectively referred to herein as a multi-catheter system.

When referring to such disclosed systems, devices, and methods, the term “proximal” (P) shall mean closer to the user or in a direction toward a device to be manipulated by the user outside the patient's body (e.g., a delivery handle 1010 of delivery system 1000), and the term “distal” (D) shall mean more distant from the user or in a direction toward a device that is positioned at the treatment site within the patient's body (e.g., fixation device 112). With respect to the mitral valve and tricuspid valve, “proximal” shall refer to the atrial or upstream side of the valve leaflets, and “distal” shall refer to the ventricular or downstream side of the valve leaflets.

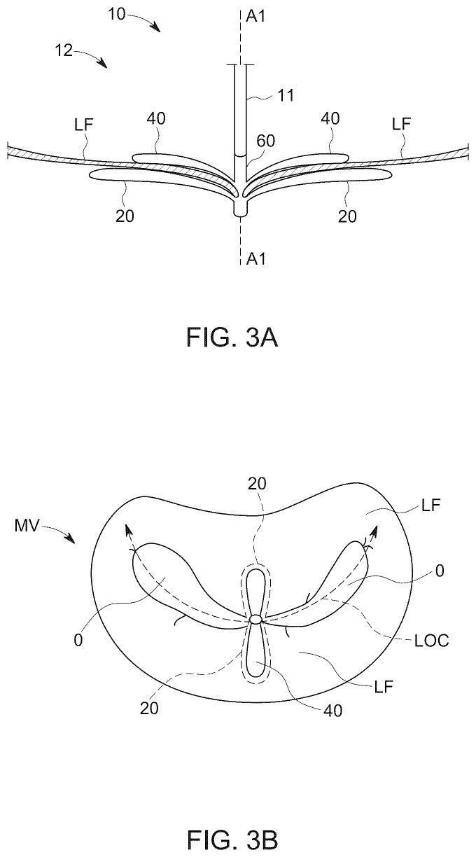

FIGS. 3A and 3B depict a fixation device 12, according to an embodiment of the present disclosure, grasping leaflets LF of an atrioventricular valve, which is illustrated as a mitral valve MV. Fixation device 12 may be releasably coupled to a distal end of a shaft 11 of a delivery system (e.g., delivery system 100) to form an interventional tool 10. Fixation device 12 may include distal elements 20 (also referred to herein as fixation elements) and proximal elements 40 (also referred to herein as gripping elements). Distal and proximal elements 20, 40 may be moveable relative to each other and may protrude radially outward relative to a longitudinal axis A1 of fixation device 12. As shown in FIG. 3A, fixation device 12 may be positionable on opposite sides of adjacent leaflets LF of the valve so as to capture or retain the leaflets LF therebetween. In this regard, proximal elements 40 may be positioned at a proximal side of the valve leaflets LF, and distal elements 20 may be positioned on a distal side of the valve leaflets LF. Proximal elements 40 may be made from cobalt chromium, nitinol, or stainless steel, for example, and distal elements 20 may be made from cobalt chromium or stainless steel, for example.

Fixation device 12 may be releasably coupled to shaft 11 such that it can be detached and left behind as an implant to hold the leaflets LF together in the coapted position. In this regard, fixation device 12 may be delivered to a target valve percutaneously using any one of a number of different approaches, such as via a transfemoral, a transapical, or a transjugular approach, for example. Thus, in one example of treating MVR, fixation device 12 may be delivered to the deficient mitral valve MV using a transfemoral approach in which fixation device 12 is guided through the inferior vena cava IVC (see FIG. 1A), across the interatrial septum S, and into left atrium LA where fixation device 12 is advanced into the mitral valve MV. Also, in one example of treating TVR, fixation device 12 may be guided transfemorally through the inferior vena cava IVC to the right atrium RA where fixation device 12 is advanced to a desired position within the tricuspid valve TV.

FIG. 3B is an atrial-side view of fixation device 12 in one example of a desired orientation in relation to adjacent leaflets LF of an atrioventricular valve, such as the depicted mitral valve MV. The distal and proximal elements 20, 40 are positioned to be substantially perpendicular to the line of coaptation LOC. Thus, in the case of a mitral valve MV, fixation device 12 may be oriented perpendicular (±5 degrees) to a line of coaptation LOC between the posterior leaflet PL and anterior leaflet AL, and in the case of a tricuspid valve TV, fixation device 12 may be positioned perpendicular (±5 degrees) to a line of coaptation between the septal leaflet SL and the anterior leaflet AL, the septal leaflet SL and the posterior leaflet PL, or the anterior leaflet AL and the posterior leaflet PL, for example. Fixation device 12 may be moved roughly along the line of coaptation LOC to the location of regurgitation. The leaflets LF may be held in place so that, during diastole, the leaflets LF remain in position between elements 20, 40 surrounded by openings O (also referred to herein as orifices) which result from the diastolic pressure gradient. Advantageously, leaflets LF are coapted such that their proximal or upstream surfaces face each other in a vertical orientation, parallel to the direction of blood flow through the valve. The upstream surfaces may be brought together so as to be in contact with one another or may be held slightly apart but will preferably be maintained in the vertical orientation in which the upstream surfaces face each other at the point of coaptation. This simulates the double orifice geometry of a standard surgical bow-tie repair. Color Doppler echo will show if the regurgitation of the valve has been reduced. If the resulting flow pattern is satisfactory, the leaflets LF may be fixed together in this orientation. If the resulting color Doppler image shows insufficient improvement in valve regurgitation, fixation device 112 may be repositioned. This may be repeated until an optimal result is produced wherein the leaflets LF are held in place.

FIGS. 4A-19C depict a fixation device 112 according to another embodiment of the present disclosure. Fixation device 112 may generally include a pair of distal elements 120, a pair of proximal elements 140, a coupling member 160, an actuation mechanism 113, and a stud 131. Distal elements 120 may include elongate arms 121 in which each arm has a proximal end portion 121a, which may be rotatably connected to the coupling member 160, and a free end 121b, as best shown in FIG. 4A. Free ends 121b may each have a rounded shape to minimize interference with and trauma to surrounding tissue structures according to one example. In one example, each free end 121b defines a curvature extending about two axes 126, 127. The first axis 126 may be a longitudinal axis of each respective arm 121. Additionally, arms 121 may each include an engagement surface 125 that may also be curved about first axis 126 and may extend at least partially along a length of arm 121 to the free end 121b. Thus, in some examples, engagement surfaces 125 may each have a cupped or concave shape which may maximize contact area engagement with tissue and may assist in grasping and holding valve leaflets. Such cupped or concave shape may further allow arms 121 to nest around shaft 111 of interventional tool 110 while in the closed position to minimize the profile of device 112. Thus, arms 121 may be at least partially cupped or curved inwardly about their longitudinal axes 126 which may form a concavity extending along axis 126 which may nest proximal elements 140 when in a lowered position thereof. The second axis 127 about which each free end 121b may be curved may extend perpendicular to first axis 126, as is also shown in FIG. 4A. The curvature about this second axis 127 may be a reverse curvature located at the most distal portion of free ends 121b. In addition to the dual curvature, free ends 121b may flare outwardly at their respective longitudinal edges. It is believed that both the reverse curvature and flare help create an atraumatic configuration that minimizes trauma to the tissue engaged therewith.

In the nonlimiting embodiment depicted, a transverse width across engagement surfaces 125 (which is in the direction of second axis 127 and determines the width of tissue engaged) may be at least about 2 mm, 3-10 mm in some examples, and about 4-6 mm in some examples. In some embodiments, a wider engagement may be desired wherein the engagement surfaces 125 are larger, for example about 2 cm, or multiple fixation devices 112 may be used adjacent to each other. Arms 121 may also have a length of about 6-12 mm (defined along first axis 126), and engagement surfaces 125 may be configured to engage a length of tissue of about 4-10 mm along the longitudinal axis 126 of arms 121 according to some examples. Also, as shown in the illustrated example, each arm 121 may include a plurality of openings 128 to enhance grip and to promote tissue ingrowth following implantation.

In one example, actuation mechanism 113 may include two link members or legs 130. Legs 130 may be comprised of a rigid or semi-rigid metal or polymer such as Elgiloy®, cobalt chromium or stainless steel, however any suitable material may be used. Each leg 130 may have a first end 132, which may be rotatably joined with one of the distal elements 120 at a riveted joint 135, and a second end 134, which may be rotatably joined with stud 131, as shown in FIG. 4A. Although the depicted embodiment shows both legs 130 pinned to stud 131 by a single rivet 135, it is also contemplated that each leg 130 may be individually attached to the stud 131 by a separate rivet, pin or the like. In other embodiments of actuation mechanism 113, actuation mechanism 113 may include a base 139, and second ends 134 of legs 130 may be rotatably joined with base 169, such as by one or more riveted joints 135, as best shown in FIG. 10B. An actuator rod 170 of delivery system 1000 may be joinable with actuation mechanism 113 directly, such as via direct connection with base 139, or indirectly, such as via connection with stud 131, which itself may extend from base 139. In either of these embodiments, actuator rod 170 may be axially extendable and retractable in a proximal-distal direction to actuate actuation mechanism 113 and consequently rotate distal elements 120 between open, closed, and inverted positions, which are described further below. Additionally, coupling member 160, stud 131, and/or base 169 may comprise a center portion or center body of fixation device, for example.

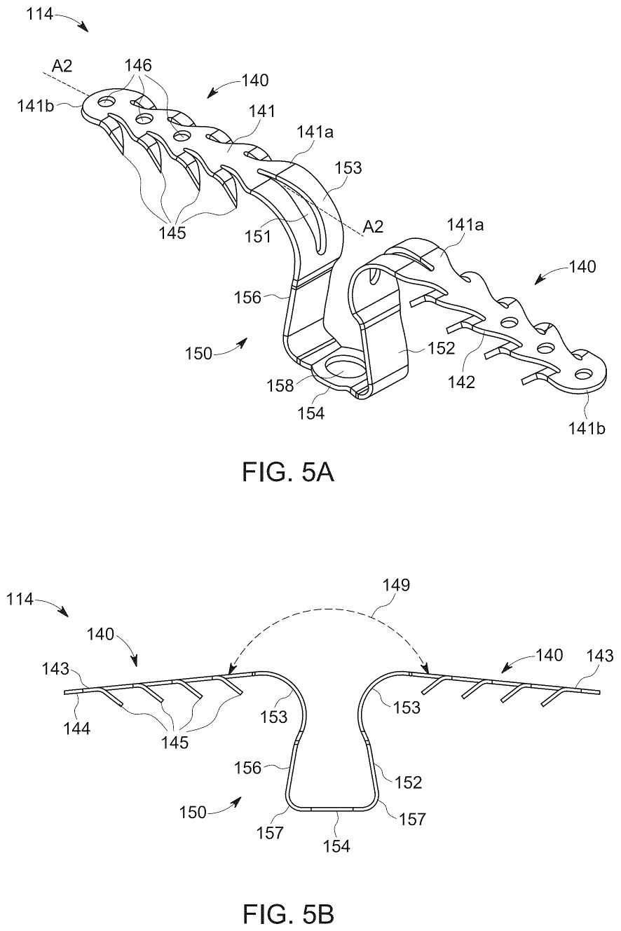

Proximal elements 140 may, in some examples, be flexible, resilient, and cantilevered from a center of fixation device 112. For example, FIGS. 5A and 5B depict a gripping device 114 according to an embodiment of the present disclosure that may generally include a pair of proximal elements 140, a base section 150, and a pair of arm bend features 153 partitioning proximal elements 140 from base section 150.

Proximal elements 140 may be in the form of elongate arms 141 that each extend along a longitudinal axis A2 from a first end portion or fixed end 141a to a second end portion or free end 141b, as shown in FIG. 5A. Each proximal element 140 may also have opposed side edges 142 that define a width transverse to the longitudinal axis A2. Such width may be less than the width of a corresponding distal element 120 such that proximal element 140 may be recessed within the concavity formed by engagement surface 125 of distal element 120 when proximal element 140 is moved into a lowered position, as described in more detail below.

Proximal elements 140 may also each have a first side or proximal side 143 and a second side or distal side 144. In one example, proximal elements 140 may include a plurality of openings 146 that may extend from proximal side 143 to distal side 144, as shown in FIG. 5A. Such openings 146 may be used to couple a proximal element line, which is discussed further below, to a proximal element 140 for raising and lowering proximal element 140. Each proximal element 140 may also include one or more frictional elements 145 extending from distal side 144. For example, each proximal element 140 may include one or more rows of frictional elements 145 where frictional elements 145 in each row may be aligned in a direction transverse to longitudinal axis A2. Frictional elements 145 in such rows may also be aligned with frictional elements 145 in other rows in a lengthwise direction thereby forming columns of frictional elements 145. For example, in the embodiment depicted in FIGS. 5A and 5B, each proximal element 145 may include four rows of two frictional elements 145. In other words, two columns of four frictional elements 145. In other embodiments, proximal elements 140 may include one to six rows of two to six frictional elements 145 per row, for example. However, in other embodiments, frictional elements 145 may be arranged in an offset relationship in a lengthwise and/or transverse direction such that at least some frictional elements 145 are not aligned with another frictional element 145 in such directions.

Frictional elements 145 may comprise frictional protrusions or tines having tapering pointed tips extending from distal side 144 of proximal elements 140. Frictional elements 145 may also be angled toward fixed end 141a of proximal element 140 which may help prevent frictional elements 145 from inadvertently snaring tissue during repositioning of fixation device 112. In one example, frictional elements 145 may be integral with or connected to a distal surface 144 of a proximal element 140 and protrude therefrom. In another example, as shown in FIG. 5A, frictional elements 145 may be formed from side edges 142, such as by cutting and bending the base material forming proximal elements 140, for example. It may be appreciated that any suitable frictional elements may be used, such as prongs, windings, bands, barbs, grooves, channels, bumps, surface roughening, sintering, high-friction pads, coverings, coatings, or a combination of these. However, it should be noted that some types of frictional elements that can be utilized may permanently alter or cause some trauma to the tissue engaged. Thus, it is preferable that frictional elements 145 be atraumatic and generally frictional rather than penetrative so as to not injure or otherwise affect the tissue in a clinically significant way.

Base section 150 may be connected to a center portion of fixation device 112 such that proximal elements 140 extend outwardly therefrom. For example, base section 150 may be coupled to coupling member 160. In the embodiment depicted, base section 150 may include a first member 152, a second member 154, and a third member 156. First and third members 152, 156 may be connected to second member 154 to form a generally U-shaped or box-shaped structure which may allow a locking mechanism (discussed below) to be positioned between first and third members 152, 156. However, other shapes may be formed, such as a V-shape, a crescent shape, or semicircular, for example. In some embodiments, first and third members 152, 156 may be connected to second member 154 via base bend features 157, for example. Also, second member 154 may include an opening 158 extending therethrough for receipt of stud 131 and/or actuator rod 170, as shown in FIG. 5A.

Arm bend features 153 may couple a respective proximal element 140 and base section 150. For example, an arm bend feature 153 can couple a proximal element 140 to first member 152 of base section 150, and another arm bend feature 153 can coupled the other proximal element 140 to third member 156 of base section 150. As shown, arm bend features 153 may form a living hinge about which proximal elements 140 may bend relative to base section 150. In this regard, arm bend features 153 may be integral with proximal elements 140 and base section 150 and may bias proximal elements 140 to a relaxed position. As illustrated in FIG. 5B, proximal elements 140 may form a relaxed angle 149 formed between proximal sides 143 of each proximal element 140. Such relaxed angle 149 is formed when proximal elements 140 are in the relaxed position and may form an angle of about 85 degrees to 200 degrees (±5 degrees). For example, proximal elements 140 may form a relaxed angle of 180 degrees in the relaxed position. In another example, proximal elements 140 may form a relaxed angle of 185 degrees in the relaxed position. Although the embodiment depicted illustrates bend features 153 as living hinges, in other embodiments bend features 153 may comprise a biased hinge that modularly connects proximal elements 140 to base section 150. For example, proximal elements 140 may be separately formed from base section 150 and modularly connected to base section 150 via arm bend features 153 which may each comprise a spring biased hinge biasing a respective proximal element 140 to the relaxed position, for example.

Arm bend features 153 may also each include an elongate opening extending 151 along the longitudinal axis A2 which may furcate each arm bend feature 153, as illustrated in FIG. 5A. Such an elongated opening 151 may have a uniform width extending along axis A2. However, in some embodiments, such as the embodiment depicted, elongate opening 151 may form a bowling-pin shape such that a width of opening 151 is narrower at one end (e.g., the end closest to free end 141b) than the other end (e.g., the end furthers from free end 141b) and is wider somewhere in between. Elongate opening 151 may also not be relegated to just arm bend feature 153 but may also extend from arm bend feature 153 to proximal element 140 and/or base body 150. The elongate opening 151 and corresponding furcation of arm bend features 153 may be configured (e.g., in size, shape, spacing, position, etc.) so as to provide the desired resiliency, fatigue resistance, and/or flexibility at the coinciding arm bend features 153.

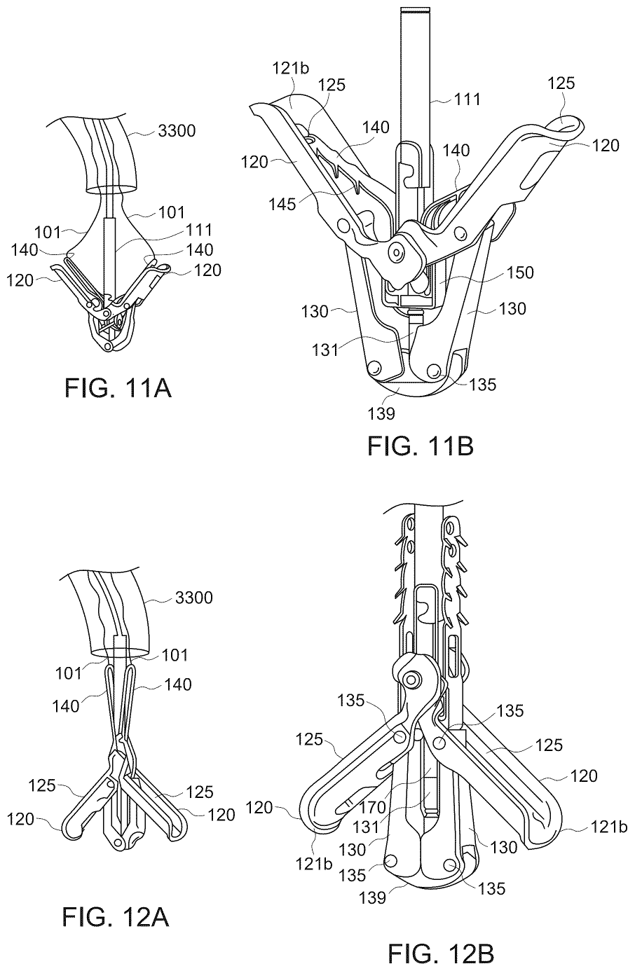

Base bend features 110 and arm bend features 112 may be configured to give gripping device 114 a bent configuration when gripping device is in a relaxed state (i.e., when proximal elements are in the relaxed position), such that when gripping device 114 is forced into a stressed state (e.g., by bending proximal elements at one or more of the base and/or arm bend features 110 and 112), gripping device 114 is resiliently biased toward the relaxed state. In the exemplary embodiment depicted, gripping device 114 may be formed from a metallic sheet of a spring-like material, such as a shape-memory metal (e.g., Nitinol) which may provide the bias of proximal elements 140 toward the relaxed position. Alternatively, elements 140 gripping device 114 could be molded from a biocompatible polymer. Each proximal element 112 may, in one example, be configured to be at least partially recessed within the concavity of the distal element 120 when no tissue is present. When fixation device 112 is in the open position, each proximal element 140 may be separated from the engagement surface 125 near free end 121b of arm 121 and may slope toward engagement surface 125 near free end 121b with the free end 141b of proximal element 140 contacting engagement surface 125, as illustrated in FIGS. 4A and 11B. This arrangement may be facilitated by the dimensions of base section 150. For example, increasing or decreasing the respective lengths of first, second, and third members 152, 154, 156 of base section 150 may increase or decrease the separation distance between a proximal element 140 and corresponding distal element 120 which may help accommodate a valve leaflet or other tissues of varying thicknesses. Further examples of gripping devices that may be utilized in fixation device 112 are described in more detail in U.S. Pat. No. 11,096,691, the disclosure of which is incorporated by reference herein in its entirety.

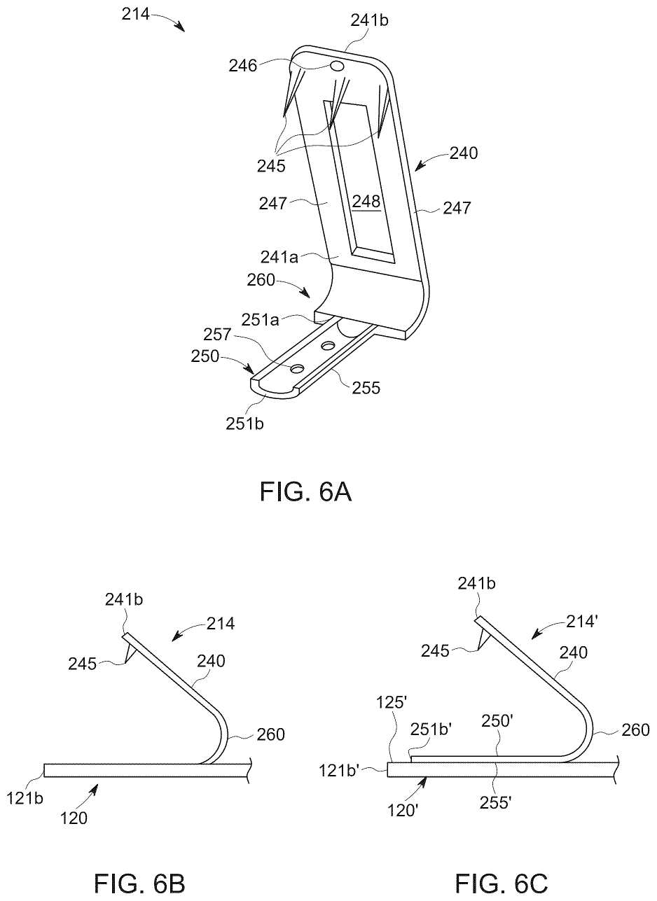

In other embodiments proximal elements may be connected to or otherwise extend from distal elements rather than from a center of fixation device, like that of fixation device 112. For example, FIGS. 6A and 6B depict a gripping device 214 according to another embodiment of the present disclosure that may generally include a first arm 240, a second arm 250, and an arm bend feature 260 partitioning first arm 240 from second arm 250. Gripping device 214 may be made from a memory-metal material, such as Nitinol, for example.

First arm 240 may constitute a proximal element of fixation device 112, like that of and as an alternative to proximal element 140 and may include one or more frictional elements 245 which may be similar to frictional elements 145 discussed above. Thus, a plurality of frictional elements 245 may extend from a distal side of first arm 240 such as in one or more rows and/or columns. In the embodiment depicted, a single row of three frictional elements 245 may be provided near a free end 241b of first arm 240. But, as mentioned above, first arm 240 may have any number of frictional elements 245, such as two, four, or six, for example. First arm 240 may also include a pair of elongate members 247 offset from each other to form a space 248 therebetween. Such space 248 may be configured to receive second arm 250, for example. Additionally, first arm 240 may include one or more openings 246, such as near free end 241b, as shown in FIG. 6A. Such opening 246 may be configured to receive a proximal element line for raising and lowering first arm 240.

Second arm 250 may be in the form of a beam or other elongate structure. Second arm 250 (also referred to herein as base section 250) may be configured to couple to a distal element 120. For example, in the embodiment depicted in FIGS. 6A and 6B, second arm 250 may be curved in a plane transverse to its longitudinal axis. For example, second arm 250 may be semi-cylindrical such that it may have a semi-circular profile. Thus, second arm 250 may have a convex surface 255 configured to conform to the cupped curvature of engagement surface 125 of a corresponding distal element 120. FIG. 6B illustrates second arm 250 coupled to proximal engagement surface 125 of distal element 120 such that it is generally recessed within distal element 120 and free ends 241b, 251b of first and second arms 240, 250 point in the general direction toward free end 121b of distal element 120. Thus, in some embodiments, second arm 250 may have a width configured to be positioned within the concavity of distal element 120 and secure to proximal engagement surface 125. In other embodiments, a second arm 250′ of an alternative gripping device 214′ may not be concave and may instead have a planar surface corresponding to a planar engagement surface 125′ of an alternative distal element 120′ and secured thereto, as illustrated in FIG. 6C. In further embodiments, distal element 120 may include a recess or pocket for receipt and securement of second arm 250, such as in a press-fit manner, for example. Second arm 250 may be secured to distal element 120 in any number of ways, such as via one or more sutures, welding, press-fit, fastener (e.g., rivet or screw) or the like. For example, a rivet, screw, or suture may pass through one or more openings 257 in second arm 250 and into distal element 120. A tissue fixation device, such tissue fixation device 112, may include a pair of gripping devices 214 with one coupled to each distal element 120 as mentioned above.

Arm bend feature 260 may be coupled to a fixed end 241 of first arm 240 and a fixed end 251a of second arm 250 such that first and second arms 240, 250 extend in the same general direction and may form a V-shape when first arm 240 is in an exemplary open or raised position, as illustrated in FIGS. 6B and 6C. As shown, arm bend feature 260 may form a living hinge about which first arm 240 may bend relative to second arm 250. In this regard, arm bend feature 260 may be integral with first arm 240 and second arm 250 so as to form a monolithic structure and may bias first arm 240 to a relaxed position. Such relaxed position may include second arm 250 extending through space 248 between elongate members 247 of first arm 240 to form an X-shape. However, it should be noted that such position can generally only be achieved when gripping device 214 is not coupled to distal element 120 as the presence of distal element 120 would prevent second arm 250 from passing into space 248. It should also be appreciated that in some embodiments of gripping device 214, arm bend feature 260 may be a spring loaded or otherwise biased hinge coupling separately formed first and second arms 240, 250.

Fixation device 114 may also have a covering 117, as shown in FIG. 4B. As depicted, covering 117 may encapsulate distal elements 120 and actuation mechanism 113. Thus, engagement surfaces 125 may be covered by covering 117 which may help minimize trauma on tissues and enhance primary fixation via additional friction to assist in grasping. Additionally, covering 117 on engagement surfaces 125 may facilitate tissue ingrowth to provide for secondary fixation to ensure long-term security. Covering 117 may be loosely fitted and/or may be flexible such that device 112 can freely move to various positions all the while covering 117 conforms to the contours of the device 112 and remains securely attached thereto. It may be appreciated that the covering 117 may cover specific parts of fixation device 112 while leaving other parts exposed. For example, proximal elements 140 may be exposed, while distal elements 120 and actuation mechanism 113 may be covered. However, in some embodiments, proximal elements 140 may be covered with covering 117 to enhance grip and tissue ingrowth following implantation. Preferably, when a covering 117 is used in combination with frictional elements 145 or other frictional features, such as those extending from proximal elements 140, such features may protrude through such covering 117 so as to contact any tissue engaged by proximal elements 140.

Covering 117 may be comprised of any biocompatible material, such as polyethylene terepthalate, polyester, cotton, polyurethane, expanded polytetrafluoroethylene (ePTFE), silicon, or various polymers or fibers and have any suitable form, such as a fabric (woven or unwoven), mesh, textured weave, felt, looped or porous structure. Generally, covering 117 has a low profile so as not to interfere with delivery through an introducer sheath or with grasping and coapting of leaflets or tissue. Covering 117 may alternatively be comprised of a polymer or other suitable materials dipped, sprayed, coated, or otherwise adhered to the surfaces of the fixation device 112. Optionally, a polymer coating may include pores or contours to assist in grasping the tissue and/or to promote tissue ingrowth. Any of the coverings 117 may optionally include drugs, antibiotics, anti-thrombosis agents, or anti-platelet agents such as heparin, COUMADIN® (Warfarin Sodium), to name a few. These agents may, for example, be impregnated in or coated on the coverings 117. These agents may then be delivered to the grasped tissues surrounding tissues and/or bloodstream for therapeutic effects.

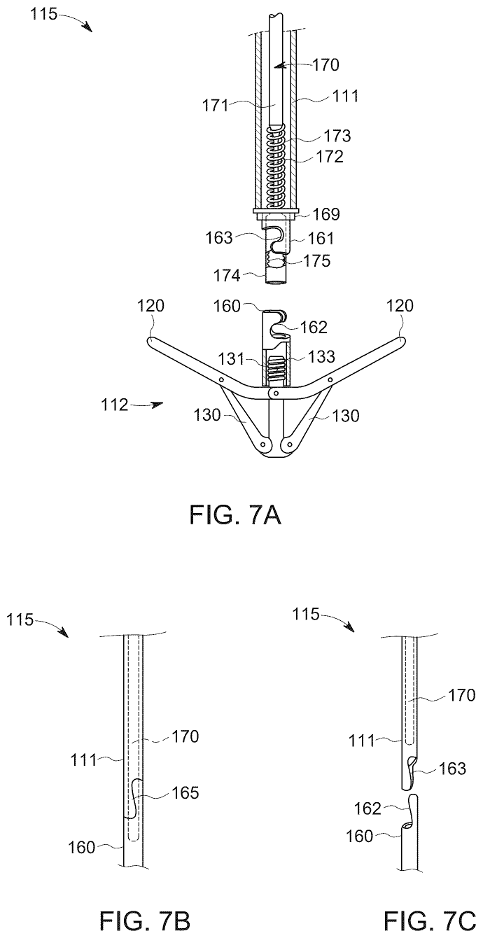

FIGS. 7A-7C depict an exemplary coupling system 115 between fixation device 112 and delivery system shaft 111. As mentioned above, once the leaflets of a target valve are coapted in the desired arrangement, fixation device 112 may then be detached from delivery system 1000 and left behind as an implant to hold the leaflets together in the coapted position. Such detachment may occur between coupling member 160 of fixation device 112 and a distal end of delivery shaft 111. Thus, coupling member 160 may be configured to be releasably coupled to shaft 111. Coupling member 160 may be disposed at a center of fixation device 112 and may extend proximally along it's the longitudinal axis of fixation device 112. In the coupling system 115 depicted, shaft 111 may form a tubular upper shaft with a first mating surface 163 formed at a distal end thereof, and coupling member 160 may form a detachable lower tubular shaft with a second mating surface 162 formed at a proximal end thereof. Mating surfaces 162, 163 may be correspondingly shaped so that they interlock and form a joining line 165 when merged together, as shown in FIG. 7B. In this regard, mating surfaces 162, 163 may have any shape or curvature which allows or facilitates interlocking and later detachment. For example, in the depicted embodiment, mating surfaces 162, 163 define a joining line 165 with an S-shaped curvature.

Coupling system 115 may also include actuator rod 170 and stud 131 (or alternatively base 139) such that fixation device 112 may also be releasably coupled to delivery system 1000 via connection between actuator rod 170 and stud 131. When shaft 111 is coupled to coupling member 160, they may collectively form an axial channel. Actuator rod 170 may pass through this channel to bridge the joining line 165, as shown in FIG. 7B. Actuator rod 170 may comprise a proximal extremity 171, a distal extremity 172, and a joiner 174. Distal extremity 172 may be smaller in diameter than proximal extremity 171 and may be optionally surrounded by a coil 173 which may serve to bias joiner 174 in a proximal direction. However, in some embodiments, actuator rod 170 may not have coil 173 or proximal and distal extremities 171, 172 of differing diameters. Joiner 174 may be removably coupled with stud 131 of fixation device 112 via any one of various possible release mechanisms. For example, in the embodiment depicted, joiner 174 may be threadedly connected to stud 131 of fixation device 112. In this regard, joiner 174 may have internal threads 175 which mate with external threads 133 on stud 131. Alternatively, joiner 174 may have external threads which mate with internal threads of stud 131. As described previously, stud 131 may be connected with distal elements 120 so that advancement and retraction of stud 131, by means of actuator rod 170, manipulates distal elements 120. It is also contemplated that joiner 174 may be directly threadedly engaged with base 139 where no stud 131 is provided. Once detachment of fixation device 112 is desired, actuator rod 170 may be rotated until threads 175 of joiner 174 disengage threads 133 of stud 131. Actuator rod 170 may then be retracted to a position above mating surfaces 162, 163 which in turn allows coupling member 160 to separate from shaft 111 along joining line 165, as illustrated in FIG. 7C.

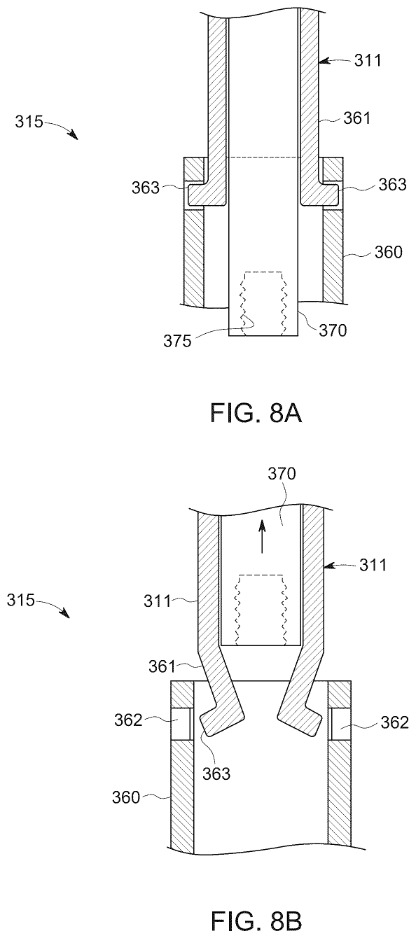

FIGS. 8A and 8B illustrate an alternative example of a coupling system. In this exemplary coupling system 315, shaft 311 of the delivery system (e.g., delivery system 1000) may be releasably coupled with coupling member 360 via a detent mechanism, for example. In this regard, shaft 311 may form an upper tubular shaft with detent mechanism features and coupling member 360 may form a lower tubular shaft with detent mechanism features configured to releasably connect with the detent mechanism features of shaft 311. In the embodiment depicted, the detent mechanism may include one or more spring arms 361 integrally formed on shaft 311 and one or more receptacles 362 sized to receive spring arms 361 within coupling member 360. However, shaft 311 may include receptacles 362, while coupling member 360 may include spring arms 361, for example. As shown, spring arms 361 may have a flange-like engagement element 363 at a distal end thereof and are preferably biased inwardly, i.e., toward an interior shaft 311, as shown in FIG. 8B. Receptacles or apertures 362 may be configured to receive and mate with respective engagement elements 363 of spring arms 361, as shown in FIG. 8A. Receptacles 362 may extend all the way through the wall of coupling member 360 and may be sized to snuggly fit both engagement elements 362. A snuggly fitting rod (such as actuator rod 370) may extend through shaft 311 and coupling member 360 and may outwardly deflecting the inwardly biased spring arm(s) 361 such that the engagement elements 363 are pushed into respective engagement with a corresponding receptacle 362 thereby coupling the shaft 311 to coupling member 360, as shown in the example of FIG. 8A. When desirable to detach fixation device 112 from delivery system 1000, actuator rod 370 may be retracted to a position above spring arm(s) 361 and engagement features 363 thereof. This allows the inwardly biased spring arms 361 and corresponding engagement elements 363 to disengage from receptacles 362 thereby detaching shaft 311 and coupling member 360. As mentioned above, actuator rod 370 may be threadedly engaged to stud 131. Thus, actuator rod 370 may first be rotated to unthread its threads 375 from stud 131 and then retracted to release coupling member 360 according to an example of the disclosure.

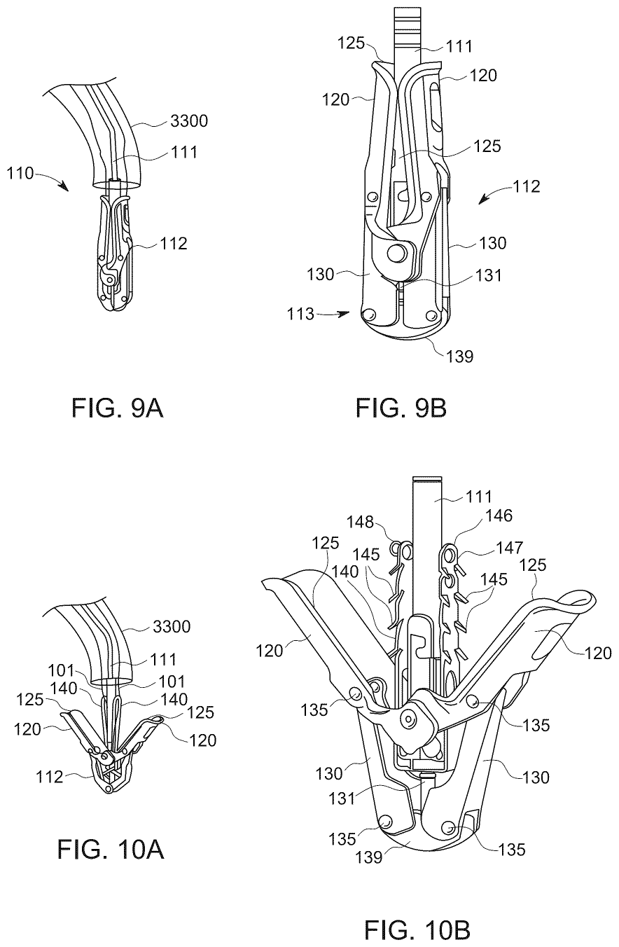

As mentioned above, fixation device 112 may, in one example, be actuated through multiple positions within a mammalian body during a transcatheter procedure such as by extending and retracting actuator rod 170 when coupled to stud 131 and/or base 139. FIGS. 9A-9B, 10A-10B, 11A-11B, 12A-12B, and FIGS. 13A-13B illustrate several of these possible positions and in a sequence that may be utilized during a transcatheter procedure.

FIGS. 9A and 9B depict fixation device 112 in an example of a closed position or delivery position. Fixation device 112 may assume the closed position when being delivered through a guide catheter or sheath 3300 of steerable guide system 5, as shown in FIG. 9A. In the closed position, the opposed pair of distal elements 120 may be positioned so that engagement surfaces 125 thereof face each other. The cupped or concave shape of each arm 121 in this example allows arms 121 to surround shaft 111 and optionally contact each other on opposite sides of shaft 111. This provides a low profile for fixation device 112 so that it is readily passable through catheter 3300 and through any anatomical structures, such as those within the cardiovascular system.

FIGS. 10A-10B depict fixation device 112 in an example of an open position. Fixation device 112 may assume the open position for capturing and grasping leaflets of a heart valve. In an open position, distal elements 120 may be rotated so that engagement surfaces 125 thereof face a first direction such that engagement surfaces 125 are disposed at an acute angle relative to shaft 111. For example, the acute angle formed between each engagement surface 125 and shaft may be 45 degrees to 90 degrees. Stated differently, in the open position, engagement surfaces 125 of distal elements 120 may be oriented 90 degrees to 180 degrees relative to each other. However, it is generally preferable for arms to be positioned 120 degrees relative to each other (and 60 degrees relative to shaft 111) for capturing leaflets. Movement of fixation device 112 from the closed position to the open position may be achieved by advancing stud 131 distally relative to coupling member 160 by distally advancing actuator rod 170. Conversely, fixation device 112 may be moved from the open position to the closed position by retracting actuator rod 170 and retracting stud 131 proximally, according to one example of the disclosure.

As shown in FIG. 10B, proximal elements 140 (or proximal elements 240) may be in a raised or insertion position when fixation device 112 is in the open position to facilitate insertion of leaflets between distal and proximal elements 120, 140 for their capture. Proximal elements 140 are, in one example, biased toward distal elements 120. In this regard, proximal elements 140 may be moved inwardly toward shaft 111 and held against shaft 111 with the aid of proximal element lines 101 which can be in the form of sutures, wires, nitinol wire, rods, cables, polymeric lines, or other suitable structures, as shown in FIG. 10A. Thus, FIGS. 10A and 10B depict fixation device 112 in an insertion configuration in which proximal elements 140 are in a raised position and distal elements 120 are in an open position.

Once fixation device 112 has been positioned in a desired location against the valve leaflets, the leaflets may then be captured between proximal elements 140 and distal elements 120. FIGS. 11A and 11B illustrate fixation device 112 in an example of such a position. Here, proximal elements 140 are lowered toward engagement surfaces 125 so that proximal elements 140 are in a lowered or capture position, and the leaflets are held between distal and proximal elements 120, 140. Proximal elements 140 are, in one example, lowered into the lowered position while distal elements 120 remain in the open position. Thus, fixation device 112, as shown in FIGS. 11A and 11B is in an example of a capture configuration which may be similar to the insertion configuration of FIGS. 10A and 10B, but with the difference being that proximal elements 140 are now lowered toward distal elements 120 by releasing tension on proximal element lines 101 to compress the leaflet tissue therebetween. At any time, the proximal elements 140 may be raised and the distal elements 120 adjusted or inverted to reposition fixation device 112 if regurgitation is not sufficiently reduced according to one example of the disclosure.

FIGS. 12A-12B depict an example of an inverted position of fixation device 112. Fixation device 112 may assume the inverted position to aid in repositioning or removal of fixation device 112. In one example of the inverted position, distal elements 120 may be further rotated from the open position, which may be achieved by advancing stud 131 further relative to the open position, so that the engagement surfaces 125 of distal elements 120 face outwardly, and free ends 121b point distally. Additionally, in some examples, engagement surfaces 125 of each arm 121 may form an obtuse angle relative to shaft 111. For example, the obtuse angle formed between each engagement surface 125 and shaft 111 may be 135 degrees to 180 degrees. Stated differently, in the inverted position, engagement surfaces 125 of distal elements 120 may be oriented 270 degrees to 360 degrees relative to each other.

Also, as shown in FIG. 12B, in one example proximal elements 140 are in their raised position against shaft 111 while distal elements 120 are in the inverted position by exerting tension on the proximal element lines 101. Thus, a relatively large space may be created between proximal and distal elements 140, 18 for repositioning. In addition, the inverted position allows withdrawal of the fixation device 112 through the valve while minimizing trauma to the leaflets. Engagement surfaces 125 provide an atraumatic surface for deflecting tissue as the fixation device is retracted proximally. It should be further noted that tines 145 of proximal elements 140 may, in some examples, be angled slightly in the distal direction (away from the free ends of the proximal elements 140), reducing the risk that tines 145 will catch on or lacerate tissue as fixation device 112 is withdrawn and while proximal elements 140 are in the raised position.

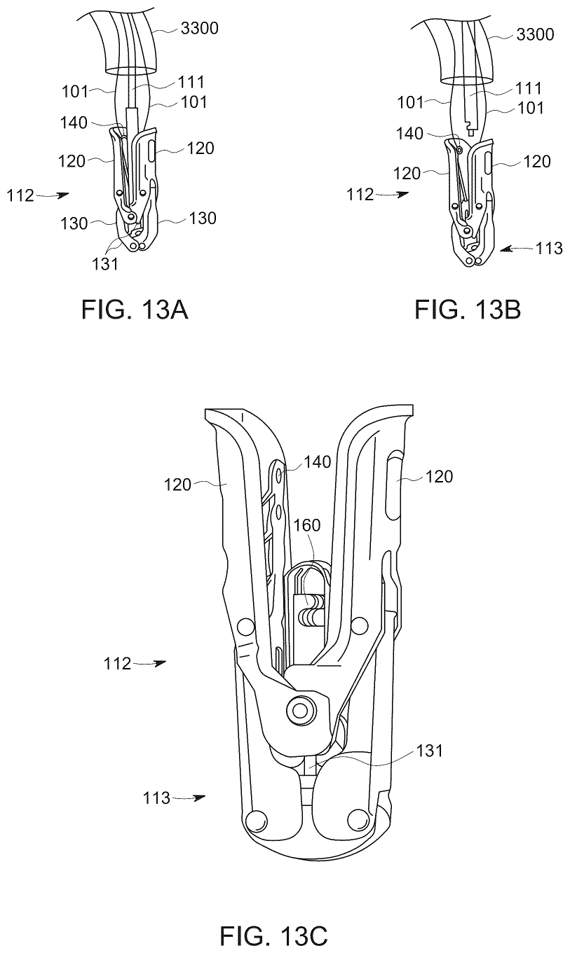

After the leaflets have been captured between distal and proximal elements 120, 140, distal elements 120 may be returned to or toward the closed position where they may be locked in place. An example of such locking is described further below. FIG. 13A illustrates fixation device 112 in the closed position wherein the leaflets (not shown) are captured and coapted. In one example, this is achieved by retraction of the stud 131 proximally relative to coupling member 160 so that the legs 130 of the actuation mechanism 113 apply an upwards force to distal elements 120 which in turn rotate distal elements 120 so that engagement surfaces 125 again face one another, similar to that of FIGS. 9A and 9B, and so that distal elements 120 rotate proximal elements 140 in a direction toward shaft 111. However, because the leaflets are captured between distal and proximal elements 120, 140, it may be desirable to keep distal elements 120 at about 20 degrees to 60 degrees relative to each other so as to limit the amount of tension and stress on the native tissue. Thus, while fixation device 112 may be returned to the closed position, such closed position may not be as closed as in the initial delivery position.

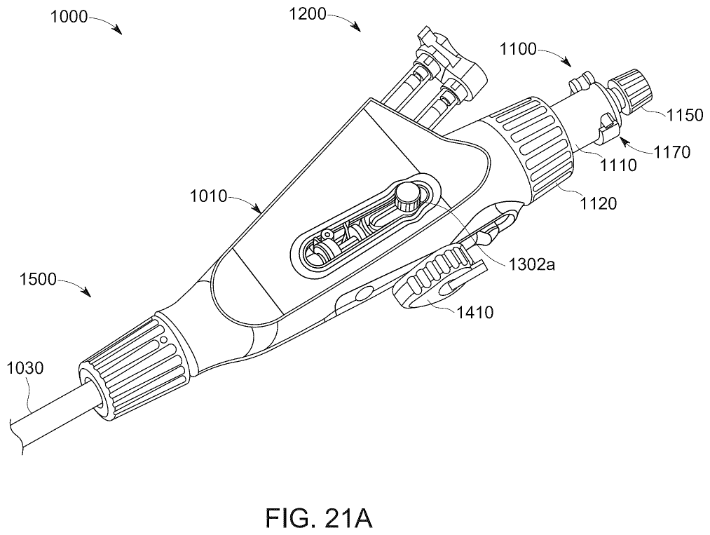

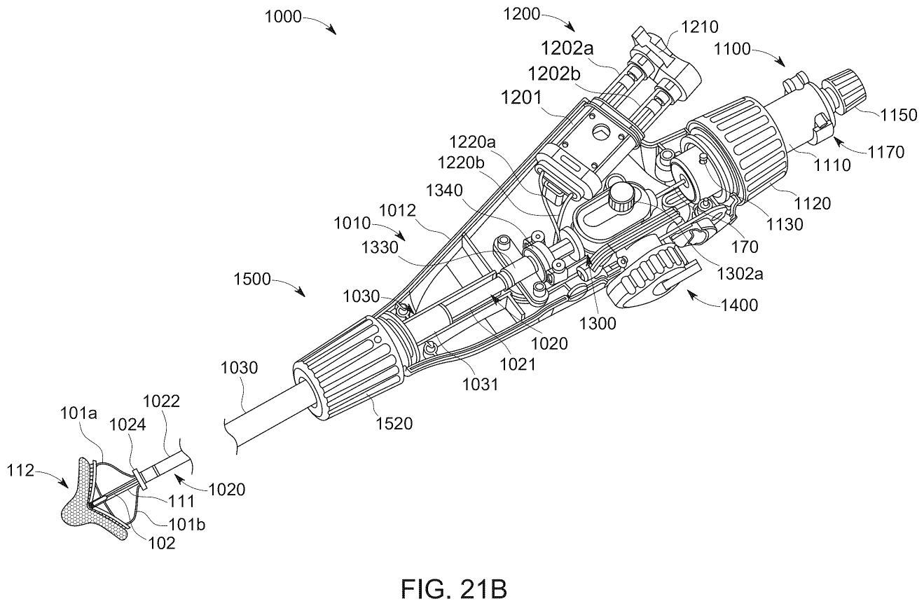

As shown in FIG. 13B, fixation device 112 may then be released from shaft 111 of delivery system 1000 while in the closed position. As mentioned, fixation device 112 may be releasably coupled to delivery system 1000 via a coupling system (e.g., coupling system 115 or 315). When the coupling structures of such coupling system are released, proximal element lines 101 may remain attached to proximal elements 140 following detachment to function as a tether to keep the fixation device 112 connected with the delivery catheter 1020 (see FIG. 21B) for reconnection and repositioning. However, in other embodiments, proximal elements lines 101 may be released prior to release of fixation device 112 or concurrently with the release of fixation device 112, as described in more detail below.

FIG. 13C illustrates a released fixation device 112 in an example of a closed position. As shown, coupling member 160 remains separated from shaft 111 of delivery system 1000, and proximal elements 140 are deployed so that tissue (not shown) may reside between proximal elements 140 and distal elements 120.

As mentioned above, proximal element lines or actuators 101 may be releasably coupled to proximal elements 140. In some examples, proximal element lines 101 may pass through an opening in proximal elements 140, such as openings 146 and 246 in the case of proximal element 240. In other examples, eyelets, which may be formed from one or more lengths of suture, may be coupled to proximal elements 140 and proximal element lines 101 may pass through such eyelets. Thus, proximal element lines 101 may be released from proximal elements 140 prior to, concurrent with, or after release of fixation device 112 from delivery system 1000 according to various examples.

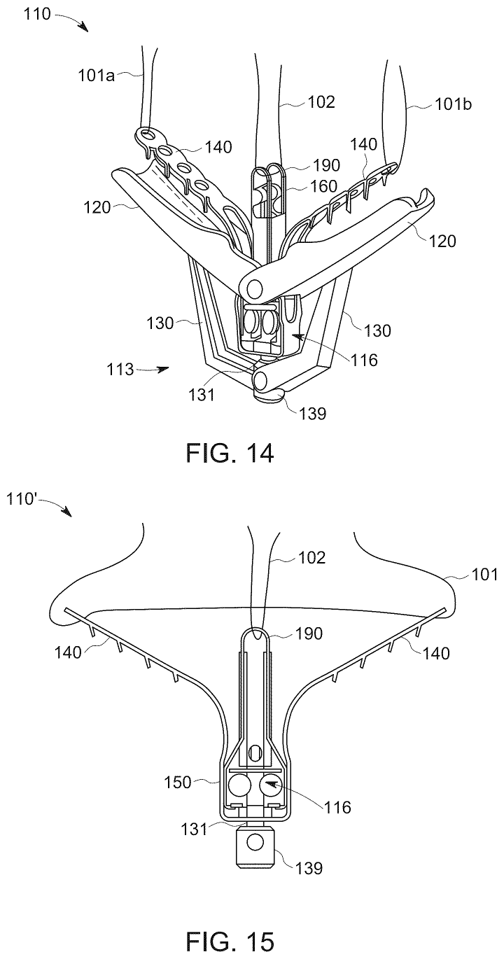

In an exemplary embodiment of interventional tool 110, as shown in FIG. 14, a plurality of proximal element lines 101a, 101b may be coupled to proximal elements 140 of fixation device 112. Each of proximal element lines 101a and 101b may be elongated flexible threads, wire, cable, sutures, or lines extending through shaft 111, looped through proximal elements 140, and extending back through shaft 111 to a delivery device handle 1010 (see FIG. 21A) of delivery system 1000. When detachment is desired, one end of each proximal element line 101a, 101b may be released from delivery system 1000, and the other end pulled to draw the free end distally through shaft 111 and through proximal element 140 thereby releasing it. Also, in this arrangement, proximal element lines 101a and 101b may be independently or concurrently manipulated so as to independently or concurrently raise and lower proximal elements 140, respectively.

In another example, interventional tool 110′ may be configured, as shown in FIG. 15 with respect to certain components thereof, such that proximal elements 140 may alternatively be supported by a single proximal element line 101 which may extend through both of the proximal elements 140. In this arrangement both proximal elements 140 may be raised and lowered concurrently by action of a single proximal element line 101. Whether proximal elements 140 are manipulated individually by separate proximal element lines 101 or jointly by a single proximal element line 101, the proximal element lines 101 may extend directly through openings (e.g., openings 146, 246) of the proximal elements 140 and/or through a layer or portion of a covering 117 on proximal elements 140, or through a suture loop/eyelet above or below a covering 117, for example.

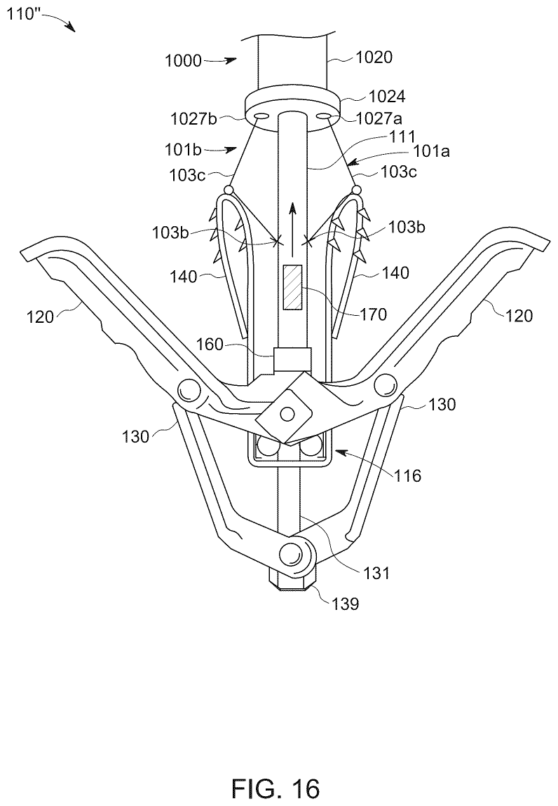

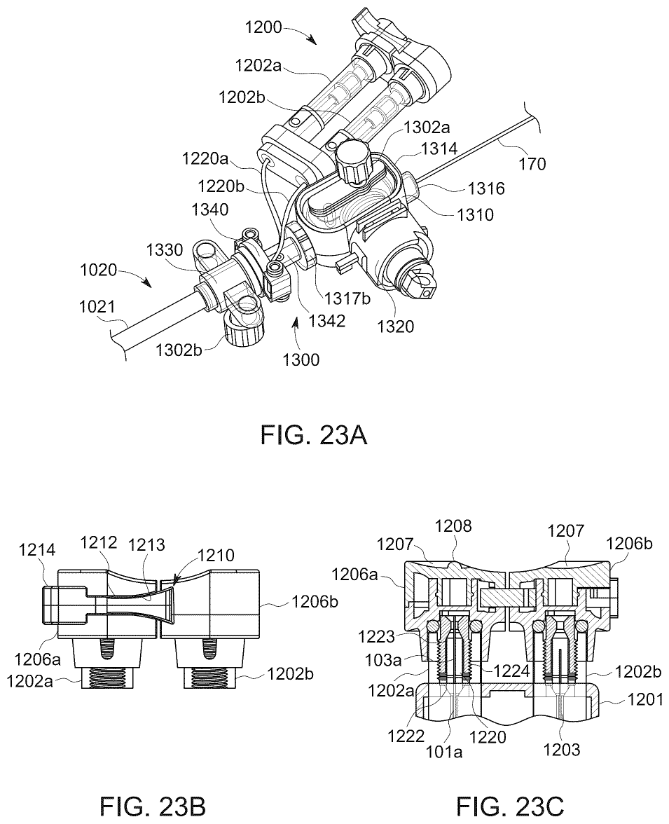

In a further example, interventional tool 110″ may be configured, as shown in FIG. 16, such that each proximal element line 101a, 101b may be releasably engaged with structures that are activated by removal of the actuator rod 170 that passes through coupling member 160 and shaft 111 such that release of proximal element lines 101a, 101b occurs concurrently with the release of fixation device 112 from delivery system 1000. Thus, in one example, which is depicted in FIG. 16, each proximal element line 101a, 101b may have a first end portion 103a (e.g., proximal end portion), which may be coupled to an actuator of a delivery system handle 1010 (see e.g., FIG. 23C), a second end portion 103b (e.g., distal end portion) which may be releasably engages to shaft 111 via actuator rod 170, and an intermediate portion 103c which may be coupled to a proximal element 140. As described above and as illustrated in FIG. 7A, stud 131 may be releasably attached to actuator rod 170 which passes through coupling member 160 and shaft 111 of delivery system 1000. In this way, actuator rod 170 is connectable with fixation device 112 and acts to manipulate fixation device 112 so as to move it through its various positions, which are described above. After the leaflets have been coapted, actuator rod 170 may be removed proximally from stud 131 which may thereby also release coupling member 160 from shaft 111, as described with respect to FIGS. 7A-7C and also FIGS. 8A and 8B with respect to coupling system 315. This action of actuator rod 170 may be utilized to release distal end portion 103b of each of proximal element lines 101a, 101b.

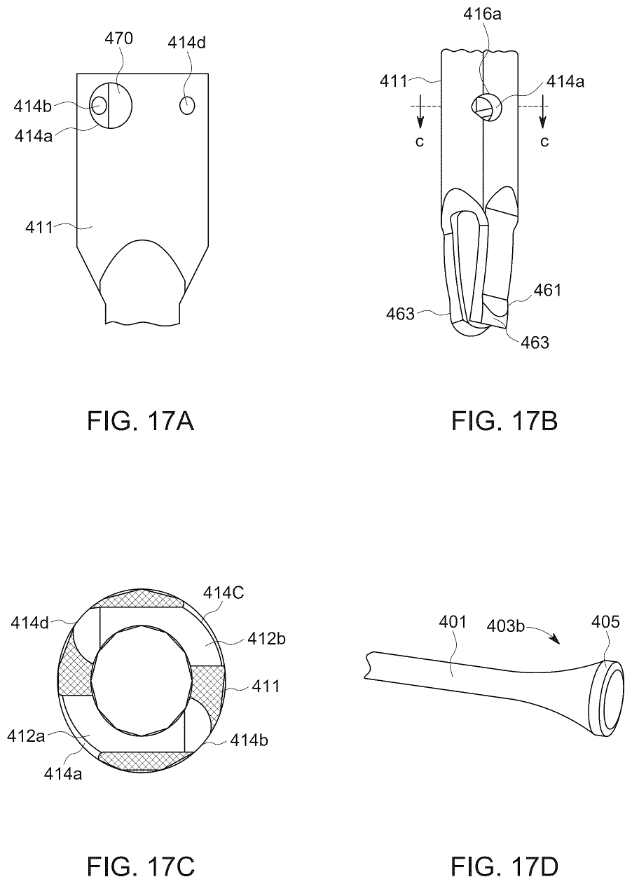

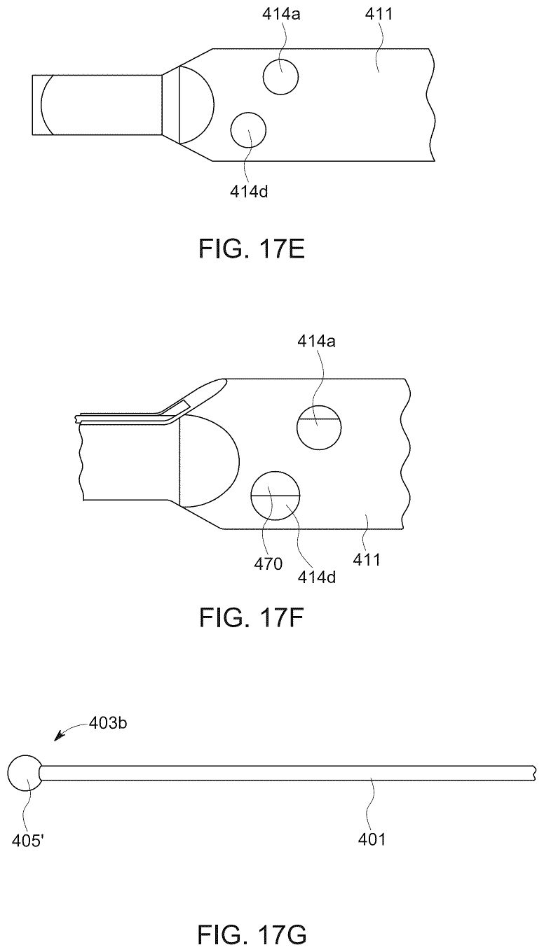

Exemplary features which may be implemented in interventional tool 110″ to facilitate release of proximal element lines 101a, 101b in this manner are shown in FIGS. 17A-17G. As depicted, an actuator rod 470 may be used as an anchor to restrict proximal movement of one or more proximal element lines 401. Proximal element line 401 has a distal end portion 403b which may include a catch element 405, for example a trumpet 405 having a cone shape (see FIG. 17D) or other shapes, such as a ball 405′ having a spherical shape (see FIG. 17G), which can be sized to be received within shaft 411. As shown in the example of FIG. 17B, shaft 411 may have spring arms 461 like that of the coupling system 315 of FIGS. 8A and 8B for releasing device 112 from shaft 411. However, shaft 411 may also have mating surfaces of FIGS. 7A and 7C. In any event, a portion of shaft 411 proximal of spring arms 461 (or mating elements 463), may have two slots 412a and 412b defined therein. Slot 412a can define holes 414a and 414b and slot 412b can define holes 414c and 414d. Holes 414a and 414c can be sized to receive catch element 405 of a pair of proximal element lines 401, respectively, therethrough and into slots 412a and 412b, respectively. Holes 414b and 414d can be sized to prevent catch element 405 of proximal element lines 401, respectively, from extending beyond slots 412a and 412b, respectively. The configuration of slots 412a and 412b and holes 414a-414d can allow for easier manufacture of the features in shaft 411. Slots 412a and 412b can be drilled to ensure that slots 412a and 412b do not pass the entire way through shaft 411. In this example configuration, catch elements 405 of proximal element lines 401 can be maintained within shaft 411 to manage the slack of proximal element lines 401.

In one example, catch element 405 of proximal element line 401 can be inserted into slot 412a through hole 414a beyond a longitudinal axis of shaft 411 and toward hole 414b, and catch element 405 of proximal element line 401 can be inserted into slot 412b through hole 414c beyond the longitudinal axis of shaft 411 and toward hole 414d prior to the insertion and coupling of the actuator rod 470 (which passes through shaft 411) with stud 131 of fixation device 112. With actuator rod 470 extending through shaft 411, actuator rod 470 may directly engage catch elements 405 of lines a plurality of proximal element lines 401 thereby preventing their movement back out along the path through which they were inserted. For example, trumpets 405 can be inhibited from being advanced through holes 414b and 414d, respectively, and can be prevented from being pulled past actuator rod 470 and through holes 414a and 414c, respectively. Accordingly, the second end portions 403b of proximal element lines 401 can be held in place relative to shaft 414. Once the actuator rod 470 is decoupled from stud 131 and subsequently retracted, movement of catch elements 405 at the distal end portions of proximal element lines 401 is no longer restricted and proximal element lines 401 are free to move. Upon proximal retraction, proximal element lines 401 can thread through holes 414a and 414c, respectively, and decouple from the proximal elements 140.

In accordance with one example of the disclosed subject matter, slots 412a and 412b can be drilled at an angle towards the distal end of shaft 411 (see FIGS. 17E and 17F), e.g., with hole 414b formed distal to hole 414a on one side, and hole 414d formed distal to hole 141c on the other side. This example configuration of slots 412a and 412b can provide easier deployment of a plurality of proximal element lines 401 and can reduce friction.

Prior to securing second end portion 403b of each proximal element line 401 with the shaft 411, each proximal element line 401 can be coupled with a respective proximal element 140, such as in the manner described above with respect to FIG. 16. Thus, when proximal element lines 401 are actuated proximally, proximal element lines 401 can move proximal elements 140 relative to distal elements 120, thereby moving proximal elements 140 between their respective raised and lowered positions.

As mentioned above, fixation device 112 optionally includes a locking mechanism (e.g., locking mechanism 116) for locking device 112 in a particular position, such as in any one of the aforementioned open, closed, and inverted positions or any position therebetween. It may be appreciated that according to various examples, locking mechanism 116 may be configured for both locking and unlocking which correspondingly allows device 112 to be both locked and unlocked. As described in more detail below with respect to various locking mechanism examples, such locking mechanisms may have components disposed between coupling member 160 and base 139 which may be configured to selectively arrest proximal-distal movement of stud 131/base 139 which consequently arrests movement of distal elements 120. Such locking mechanisms may help provide end user control of the final arm angle of fixation device 112 for tailored and optimal results for each patient. Additionally, such locking mechanisms may bring the leaflets and annulus together which may result in beneficial dimensional changes of the target valve which can prevent adverse remodeling of the heart, particularly for patients with heart failure.

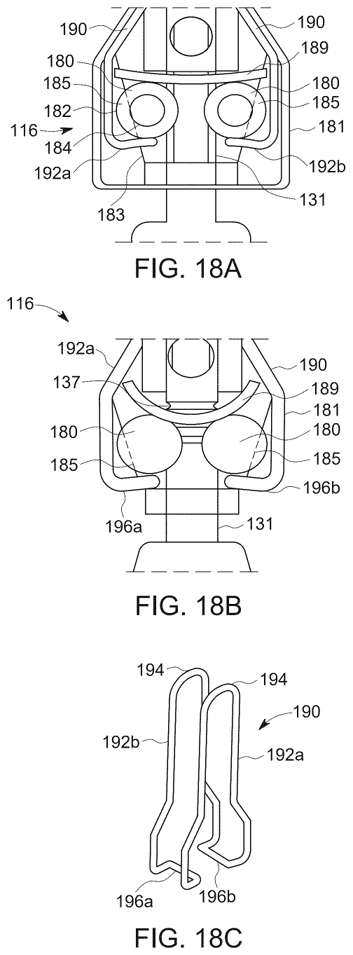

FIGS. 14, 15, and 16A-16C illustrate an embodiment of the locking mechanism 116. Locking mechanism 116 generally includes a housing 181, one or more wedging elements 180, a release harness 190, and a biasing member 189. Housing 181 may be positioned distal to coupling member 160 and may be free-floating, coupled to, or integral with coupling member 160, such as at a distal end thereof. Housing 181 may form a window 183 which may be defined at opposite sides with sloping or tapered surfaces 185 which slope inwardly toward stud 131 in a proximal to distal direction. Wedging elements 180 may be in the form of rolling elements, such as a pair of barbells, disposed on opposite sides of stud 131 and between sloping surfaces 185, as shown in FIGS. 18A and 18B. Each barbell 180 may have a pair of generally cylindrical caps 182 and a shaft 184 therebetween, as illustrated in the barbell cross-section of FIG. 16A. Barbells 180 and stud 131 are preferably comprised of cobalt chromium or stainless steel, however any suitable material may be used. Biasing member 189 may be a spring, such as a leaf spring, for example, and may be positioned at a proximal end of housing 181 between sloping surfaces 185 and proximal to barbells 180 such that spring 189 bears on barbells 180 and biases them in a distal direction. Thus, when barbells 180 are pushed distally by spring 189, they are correspondingly pushed inwardly and wedged against stud 131 by sloping surfaces 185, as illustrated by FIG. 18A, which depicts barbells 180 in a proximal and unlocked position, and FIG. 18B, which depicts barbells 180 in a distal and locked position.

As shown in FIGS. 14, 15, and 18C, release harness 190 may be in the form of a ridged wire or rod that may extend proximally from stud 131 toward a proximal end of fixation device 112 and at opposite sides thereof. In this regard, release harness 190 may form a first portion or front portion 192a and a second portion or rear portion 192b. Each of first and second portions 192a, 192b may include a crest or closed proximal end 194 through which a lock line 102 may be threaded and engaged, as described below. Release harness 190 may also form hooked distal ends 196a, 196b which may extend between first and second portions 192a, 192b and between sloping surfaces 185 and stud 131, as shown in FIGS. 18A and 18B. Thus, hooked ends 196a and 196b may be moveable proximally-distally within window 183 formed between sloped surfaces 185 and stud 131. Additionally, hooked ends 196a and 196b may be positioned distal of barbells 180 such that pulling up on harness 190 moves hooked ends 196a, 196b proximally so as to push the respective barbells 180 against the bias of spring 189 and move them to their unlocked position.

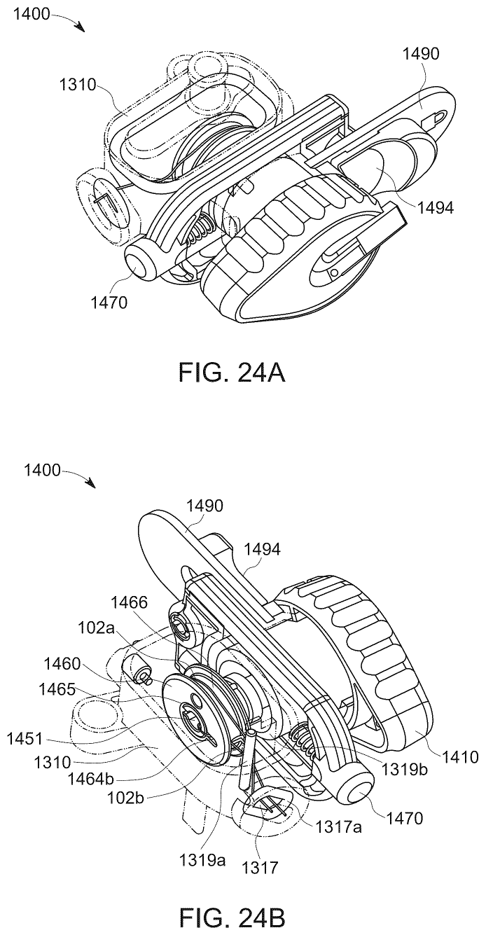

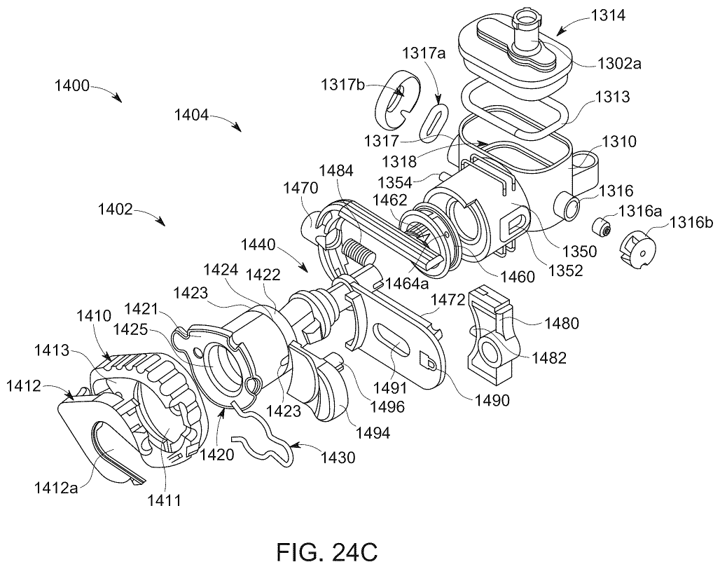

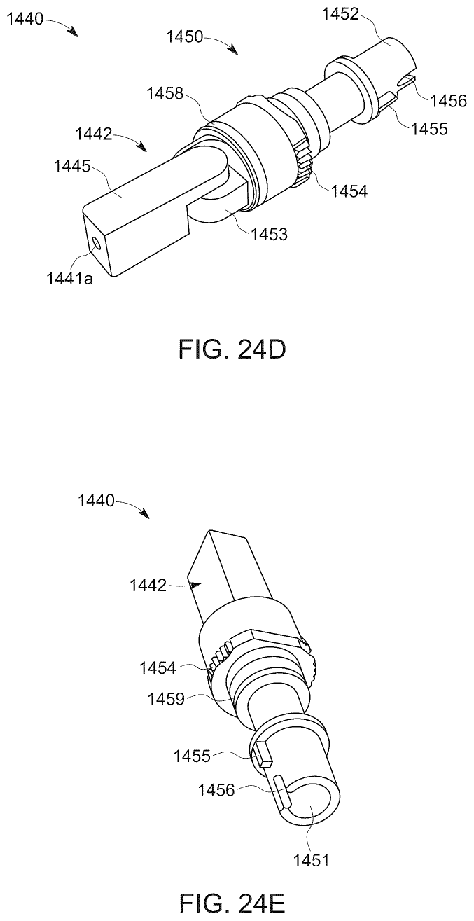

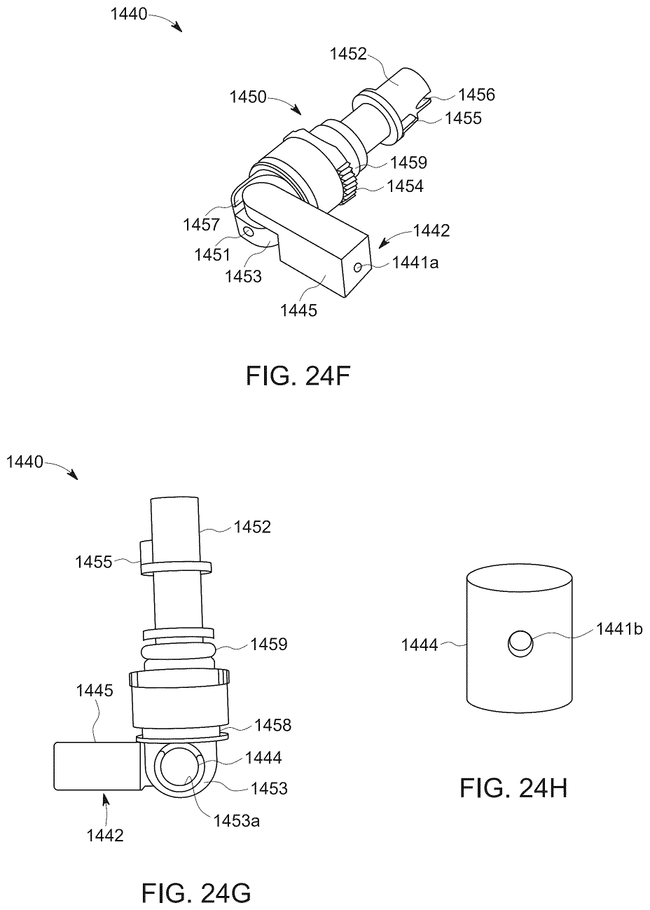

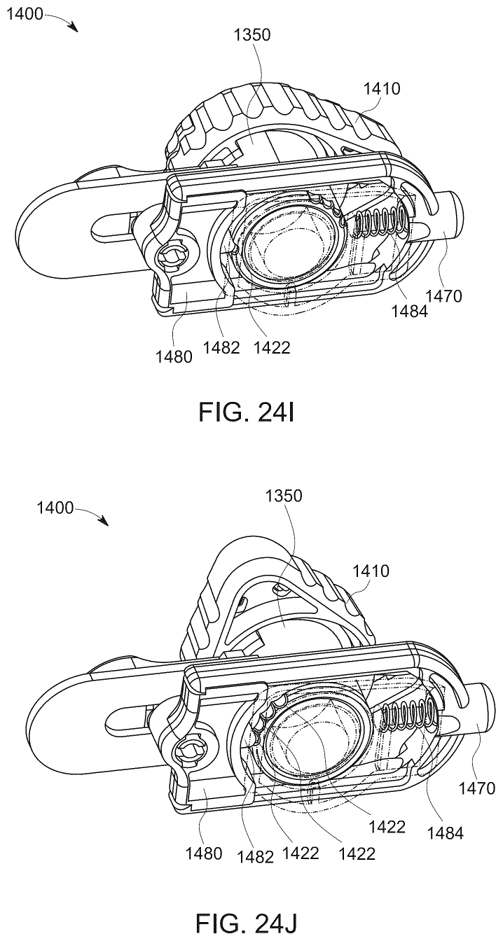

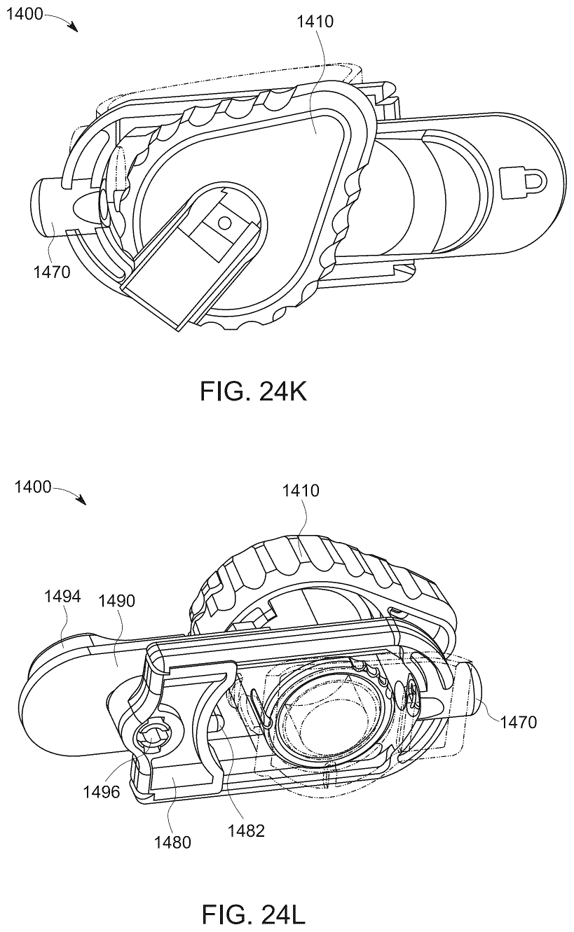

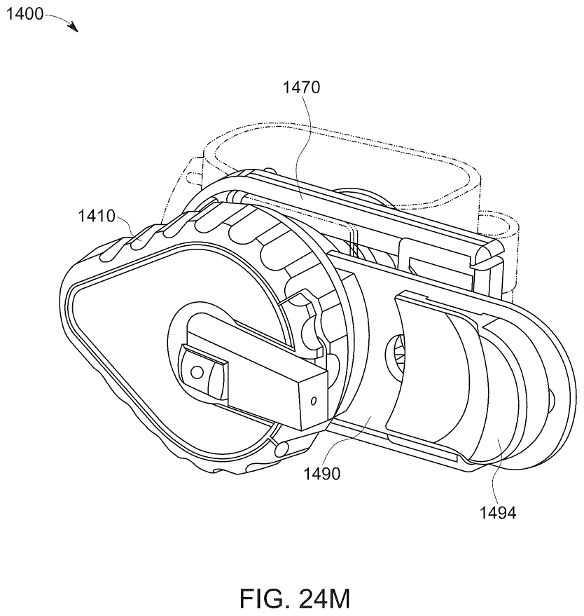

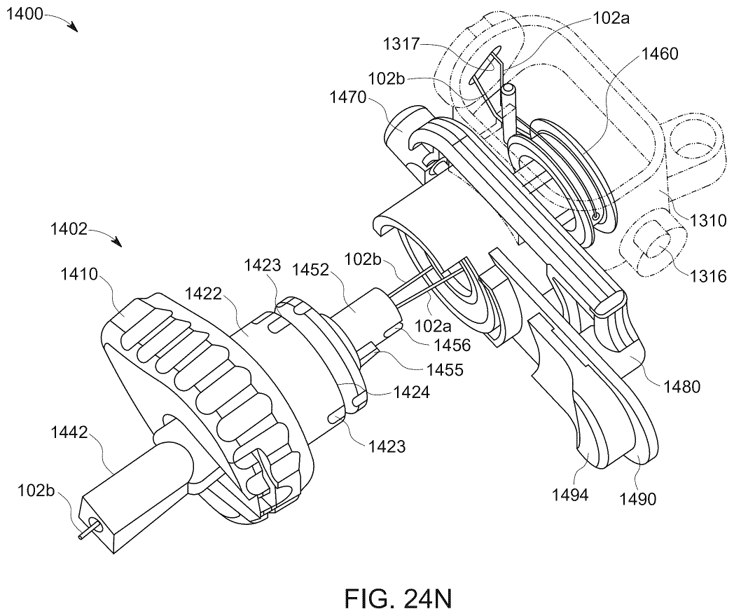

Movement of harness 190 may be performed by one or more lock line 102 which may be coupled to harness 190 by such as by threading lock line 102 through and engaging one or more of proximal ends 194 of first and second portions 192a, 192b thereof, as shown in FIGS. 14 and 15. Such lock line 102 may have a first end 102a fixedly secured to delivery system handle 1012 of delivery system 1000 and a second end 102b releasably secured to delivery system handle 1012 (see e.g., FIG. 24N), as described in more detail below. In this regard, tension can be selectively applied to lock line 102 to unlock and lock locking mechanism 116. Also, lock line 102 can be released from release harness 190 prior to, concurrently with, or after release of fixation device 112 from delivery system 1000 which may be achieved by releasing the second end 102b from delivery system handle 1010 and pulling lock line 102 and its second end through shaft 111. Lock line 102 may be comprised of any suitable material, typically wire, nitinol wire, cable, suture, or thread, to name a few. In addition, lock line 102 may include a coating, such as parylene. Parylene is a vapor deposited pinhole free protective film which is conformal and biocompatible. It is inert and protects against moisture, chemicals, and electrical charge.

When an upwards force is applied to harness 190 by the lock line 102, hooked ends 196a, 196b may raise barbells 180 against spring 189, as shown in FIG. 18A. This may draw barbells 180 up along sloping surface 185 which unwedges barbells 180 from against stud 131. In this position, stud 131 is free to move. Thus, when lock line 102 is tensioned to raise or lift harness 190, locking mechanism 116 is in an unlocked position wherein stud 131 is free to move actuation mechanism 113 and therefore distal elements 120 to any desired position. Releasing tension in lock line 102 may, on the other hand, transition the locking mechanism 116 to a locked position, as shown in FIG. 18B. Thus, by releasing the upwards force on barbells 180 by hooked ends 192a, 192b, spring 189 forces barbells 180 downwards and wedges barbells 180 between a sloping surface 185 and stud 131. This restricts motion of stud 131, which in turn locks actuation mechanism 113 and therefore distal elements 120 in place. In addition, stud 131 may include one or more grooves or indentations 137 which may receive shaft 184 of each barbell 180. This may provide more rapid and positive locking by causing barbells 180 to settle in a definite position, increase the stability of locking mechanism 116 by further preventing movement of barbells 180, as well as tangible indication to the user that each barbell 180 has reached a locking position. In addition, grooves 137 may be used to indicate the relative position of distal elements 120, particularly the distance between distal elements 120. For example, each groove 137 may be positioned to correspond with a 0.5- or 1.0-mm decrease in distance between distal elements 120. As stud 131 is moved, barbells 180 may contact grooves 137, and by counting the number of grooves 137 that are felt as stud 131 is moved, the user can determine the distance between distal elements 120 and can provide the desired degree of coaptation based upon leaflet thickness, geometry, spacing, blood flow dynamics and other factors. Thus, grooves 137 may provide tactile feedback to the user.

Locking mechanism 116 allows fixation device 112 to remain in an unlocked position when attached to delivery system 1000 during grasping and repositioning and then maintain a locked position when left behind as an implant. It may be appreciated, however, that locking mechanism 116 may be repeatedly locked and unlocked throughout the placement of the fixation device 112 if desired. Once the final placement is determined, lock line 102 may be removed and fixation device 112 may be left behind.

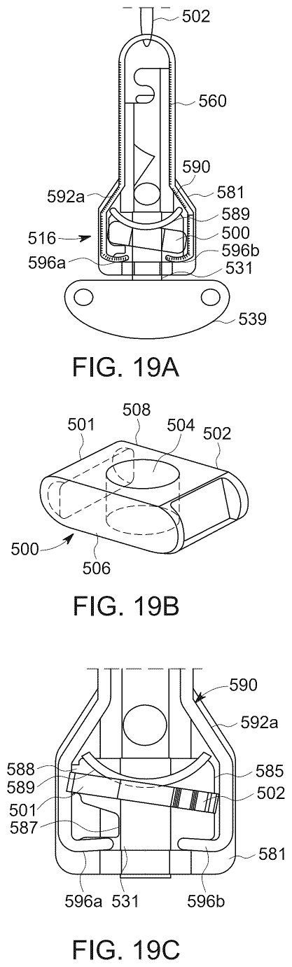

FIGS. 19A-19C depict a locking mechanism 516 according to another embodiment of the present disclosure that may be incorporated into a fixation device 112 of the disclosure. In this embodiment, locking mechanism 516 also includes a housing 581, a spring 589, a release harness 590, and a wedging element 500. However, instead of sloping surfaces 185 as present in the example of locking element 116, housing 185 may include generally parallel sidewalls 585 and may include a finger or protrusion 587 extending from one of sidewalls 585 toward stud 131, as best shown in FIG. 19C. Such finger 587 may slope in a distal direction and may define a proximal notch 588. Also, as shown in FIG. 19C, first hooked end 596a of release harness 590 may be positioned distal of finger 587.

Furthermore, wedging element may comprise a binding lever or binding plate 500. As shown in FIG. 19B, binding plate 500 may have an oblong shape that may extend lengthwise between a first end 501 and a second end 502 thereof. An aperture 504 may be formed between first and second ends 501, 502 and may extend from a top planar surface 508 through a bottom planar surface 506 of binding plate 500. Binding plate 500 may be positioned between sidewalls 406 so that stud 131 passes through aperture 504 and so that first end 501 of binding plate 500 is positioned within notch 588 proximal of finger 587, as best shown in FIG. 19C. Thus, finger 587 may be positioned between first end 501 of binding plate 500 and first hooked end 596a of released harness 590. Also, spring 589 may be positioned proximal to binding plate 500 and provide downward or distal bias thereto. Binding plate 500 and stud 131 may be comprised of any suitable material. In some embodiments, binding plate 500 may have a higher hardness than stud 131. In other embodiments, binding plate 500 may be comprised of a flexible or semi-flexible material. Such flexibility may allow slight movement of stud 131 in the proximal and distal directions, therefore allowing slight movement of distal elements 120 when locking mechanism 516 is in the locked position. This may allow fixation device 112 to adjust in response to dynamic cardiac forces.

FIGS. 19A and 19C illustrate binding plate 500 in a locked position or configuration. In this regard, spring 589 pushes binding plate 500 in a distal direction. However, because first end 501 of binding plate 501 is positioned within notch 588, axial movement of first end 501 toward a distal end of housing 581 is prohibited while axial movement of second end 502 of binding plate 500 is permitted. Thus, finger 587 obstructs first end 501 from axial movement and creates a lever type movement of binding plate 500. Moreover, finger 587 obstructs first hooked end 596a of release harness 590 from axial movement resulting in a side-to-side pivoting of release harness 590 upon tension of lock line 102. This pivoting movement correspondingly results in second hooked end 596b of release harness moving proximally and controlling movement of second end 502 of binding plate 500. As such, when an upwards force is applied to harness 590 by lock line 102, second hooked end 596b of release harness 590 raises second end 502 of plate 500 against spring 589 so that planar surfaces 506, 508 of binding plate 500 become oriented substantially perpendicular to stud 131. This aligns aperture 504 with stud 131 allowing free movement of stud 131 in the proximal-distal direction. Thus, in this state, locking mechanism 516 is unlocked wherein stud 131 is free to move actuation mechanism 113 and therefore distal elements 120 to any desired position.

Release of harness 590 by lock line 102 transitions locking mechanism 516 back to the locked position. By releasing the upwards force on second end 502 of binding plate 500, spring 589 forces second end 502 of biding plate 500 downwards, which misaligns aperture 504 relative to stud 531, and correspondingly wedges binding plate 500 against stud 131, as best shown in FIG. 19C. This arrests movement of stud 131, which in tum locks actuation mechanism 113 and therefore distal elements 120 in place. It may be appreciated that binding plate 500 may have any suitable form to function as described above. For example, plate 500 may have a variety of shapes with or without planar surfaces 506, 508 and/or the aperture 504 may be of a variety of shapes and positioned in a variety of locations, to name a few. For example, binding plate 500 may not have a through-hole, like that of aperture 504, but may rather have a notch such that binding plate 500 does not encircle stud 131 but rather partially surrounds it. Further, it may be appreciated that any number of binding plates 500 may be present. Each binding plate 500, in this regard, may provide an additional binding location which may enhance lock performance.

While the above-described nonlimiting examples of fixation device 112 may utilize a push-to-open, pull-to-close mechanism for opening and closing distal elements 120, it should be understood that a pull-to-open, push-to-close mechanism may alternatively be provided. For example, distal elements 120 may be coupled at their proximal ends to stud 131 rather than to coupling member 160, and legs 130 may be coupled at their proximal ends to coupling member 160 rather than to stud 131. In this example, when stud 131 is pushed distally relative to coupling member 160, distal elements 120 may close, while pulling on stud 131 proximally toward coupling member 160 may open distal elements 120. Regardless, the aforementioned locking mechanism examples may be configured to arrest stud to lock distal elements 120 in the desired position, as described.

It is to be understood that the fixation devices and components thereof described above are provided as examples are not to be considered as limiting to fixation devices suitable for use with other aspects of the disclosure.

FIG. 20 depicts various components of an exemplary interventional system 3 which may be utilized to implant a fixation device (e.g., fixation device 112) within a target valve. Such components may include delivery system 1000, steerable guide system 5, and stabilizer 4000. As described in more detail below, delivery system 1000 may generally include a delivery catheter handle 1010 and delivery catheter 1020. Delivery catheter handle 1010 may include a plurality of controls for steering fixation device 112 to a target valve and for controlling various features of fixation device 112, such as distal and proximal elements 120, 140, and for releasing fixation device 112 from delivery catheter 1020, for example. Additionally, steerable guide system 5 may generally include a plurality of guide catheter assemblies 2000, 3000 which may be configured to provide a conduit to help guide delivery catheter 1020 and fixation device 112 to a target valve, such as a mitral valve or a tricuspid valve, for example, and for optimal positioning of the same above a valve plane of the target valve. Stabilizer 4000 may support delivery system 1000 and steerable guide system 5 and may facilitate stable and controlled movement of fixation device 112 in-situ.

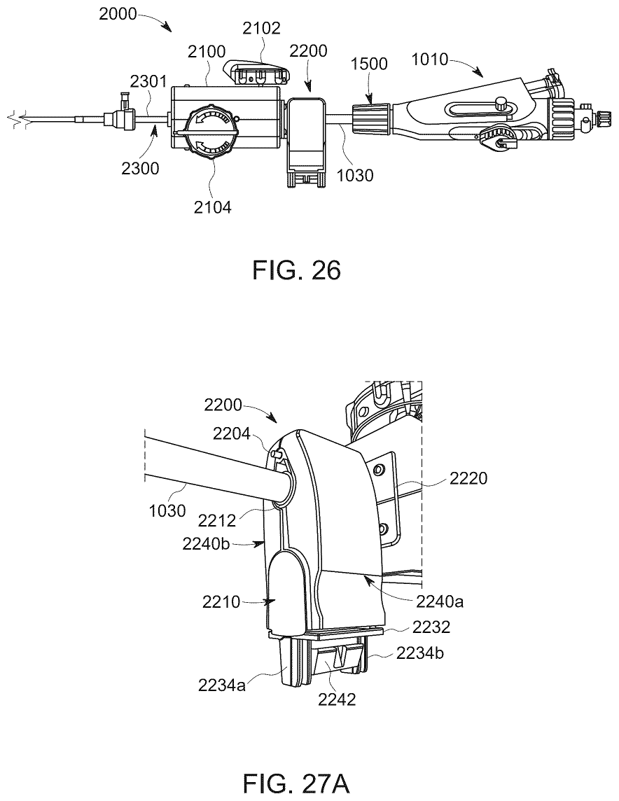

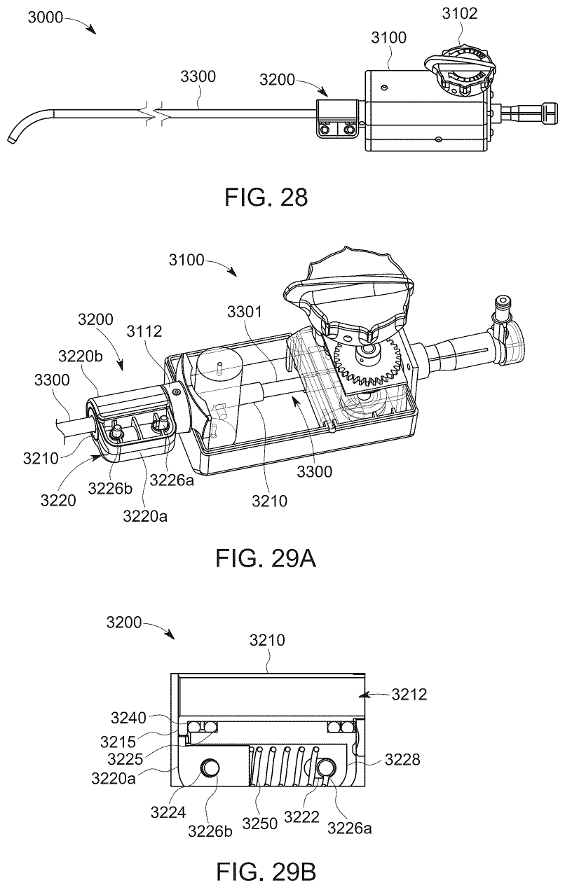

FIGS. 21A-29B depict delivery system 1000 according to an embodiment of the present disclosure. Delivery system 1000 may include a delivery catheter handle 1010 and a delivery catheter 1020 which may extend distally from delivery catheter handle 1010. The components of delivery system 1000 are configured to cooperatively function as an integrated unit for controlled and precise delivery of fixation device 112 to a target valve. Handle 1010 incorporates a plurality of control assemblies that provide the operator with tactile feedback and precise control over the positioning, deployment, and release of fixation device 112. Delivery system 1000 may also include an actuator rod 170, one or more proximal element lines 101, such as proximal element lines 101a and 101b, and one or more lock lines 102 which may extend from respective controls in handle 1010 and through delivery catheter 1020 to a fixation device 112 coupled to delivery catheter 1020, as shown in FIG. 21B and as described in more detail below.

According to one example of delivery catheter handle, delivery catheter handle 1010 may include a housing or main body 1012, a positioner assembly 1100, a gripper control assembly 1200, a fluid management assembly 1300, a lock control assembly 1400, and a delivery catheter fastening assembly 1500. Actuator rod 170 may extend from positioner assembly 1100, and positioner assembly 1100 may be configured to actuate actuator rod 170 in a proximal-distal direction for moving distal elements 120 between open, closed, and inverted positions, examples of which are described above. Positioner assembly 1100 may also be configured to release actuator rod 170 from fixation device 112 for deployment of fixation device 112. Delivery device handle 1010 may also include a deployment control system 1170 that may be coupled to positioner assembly 1100 for prevention of inadvertent deployment of fixation device 112 until desired. Proximal element lines 101a, 101b may extend from gripper control assembly 1200, and gripper control assembly 1200 may be configured to actuate proximal elements lines 101a, 101b to move proximal elements 140 between their raised and lowered positions, as described above. Lock line 102 may extend from lock control assembly 1400, and lock control assembly 1400 may be configured to actuate lock line 102 and correspondingly locking mechanism 116 or 516 of fixation device 112 between the locked and unlocked configuration, as described above. Additionally, lock control assembly 1400 may be configured to release lock line 102 from fixation device 112. Actuator rod 170, proximal element lines 101a, 101b, and lock line 102 may be directed into the fluid management assembly 1300 which itself may be coupled to delivery catheter 1020 for providing sterile fluid through one or more lumens of delivery catheter 1020. During a transcatheter procedure, it may be desirable to advance or retract delivery catheter 1020 relative to guide catheter system 5 to help position fixation device 112 within a target valve. Delivery catheter fastening assembly 1500 may be configured to selectively secure and unsecure delivery catheter 1020 so that it can be freely advanced or retracted relative to steerable guide system 5 and secured in a desired position so that delivery catheter 1020 is constrained from axially translating relative to steerable guide system 5. Each of these features are described in more detail below.

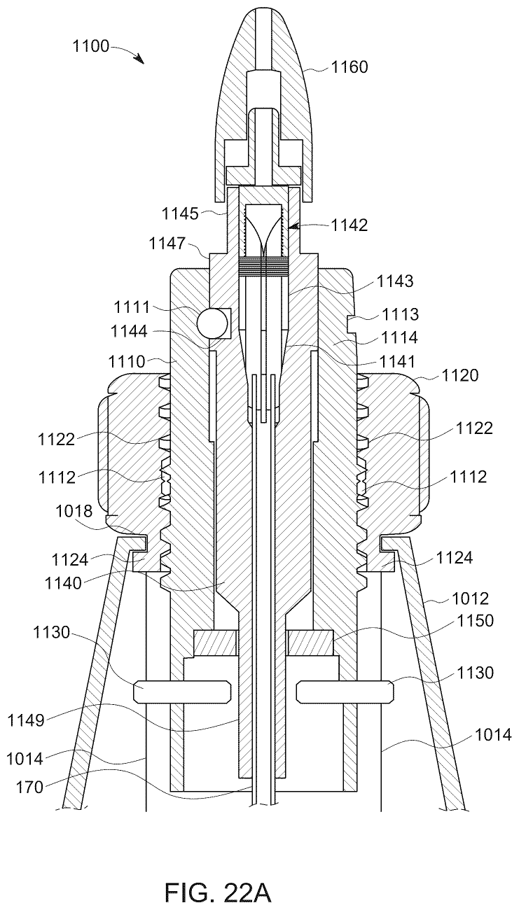

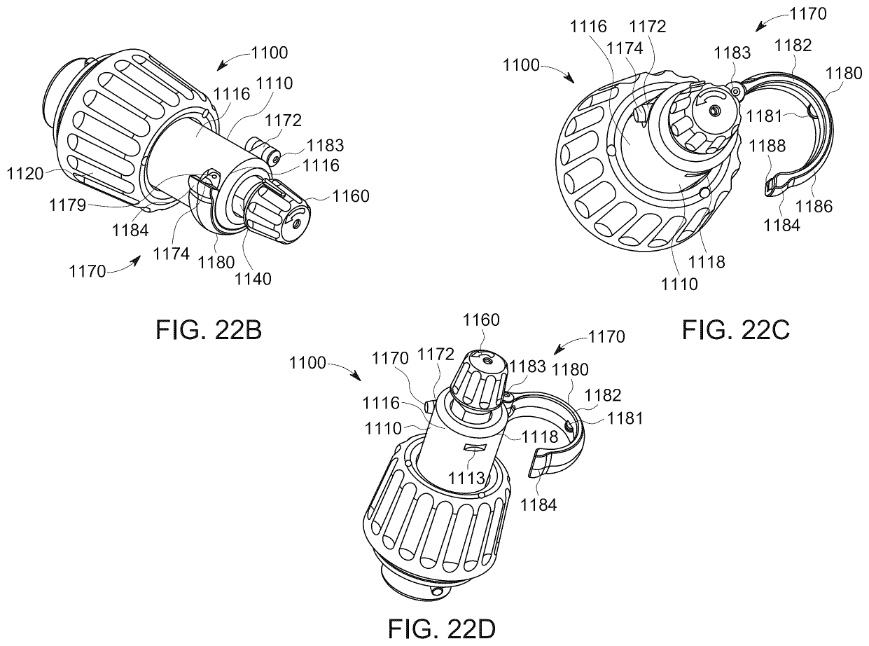

FIGS. 21B and 22A-22D depict positioner assembly 1100 according to an embodiment of the present disclosure. In one example, positioner assembly 1100 may generally include a slider 1110, an actuator knob 1120, one or more guide pins 1130, an actuator shaft 1140, a bearing 1150, and an actuator rod handle 1150.

According to various examples, slider 1110 may be substantially cylindrical and may have an axial channel 1114 that extends entirely along its length from a proximal end to a distal end thereof. A transverse opening 1111 may extend through slider 1110 near its proximal end such that it extends through opposing sides of slider 1110 and intersects channel 1114. In one example, transverse opening 1111 is offset relative to a longitudinal axis of slider 1110. Nonetheless, in such embodiment, transverse opening 1111 may be oriented generally perpendicular (±5 degrees) relative to the longitudinal axis of slider 1110. Also at the proximal end of slider 1110, a notch 1113 may extend radially inwardly into slider 1110. Such transverse opening 1111 may be configured to releasably receive a deployment pin of a deployment system, and notch 1113 may be configured to receive a protuberance of such deployment system, as described further below. At a distal end of slider 1110 in one example, a pair of guide pins 1130 may extend radially outwardly therefrom and may correspondingly engage grooves 1014 within delivery handle housing 1012. This pin 1130 and groove 1014 engagement may rotationally constrain slider 1110 relative to housing 1012 while permitting translation along housing 1012. Such guide pins 1130 may, in some examples, be orthogonal to the longitudinal axis of slider 1110 and may be press-fit to slider 1110, threaded to slider 1110, or integral with slider 1110, for example. Slider 1110 may also include external threads 1122 that extend helically along an outer surfaces thereof and that extend along at least a portion of its length, as best shown in FIG. 22A. In this regard, threads 1112 may extend along a length of slider 1110 disposed between transverse opening 1111 and guide pins 1130.

Actuator knob or actuator control 1120 may be annular such that it has a generally cylindrical exterior and interior. However, actuator knob 1120 can have other shapes, such as a conical shape, for example. As shown in FIG. 22A, in one example, actuator knob 1120 may extend circumferentially about slider 1110 and may have internal threads 1122 that engage external threads 1112 of slider 1110 such that rotating actuator knob 1120 in a first direction translates slider 1110 in a proximal direction, and rotating actuator knob 1120 in a second direction translates slider 1110 in a distal direction. Actuator knob 1120 may be rotatably connected to a proximal end of delivery handle housing 1012 while being translationally constrained. For example, actuator knob 1120 may include a circumferential lip 1124 that defines a circumferential groove that receives a corresponding lip 1018 of housing 1012, as shown in FIG. 22A. Thus, with actuator knob 1120 translationally constrained, and slider 1110 rotationally constrained via pins 1130 and grooves 1014, slider 1110 is free to translate relative to actuator knob 1120 upon its rotation.

According to one example, actuator shaft 1140 may be positioned within axial channel 1114 of slider 1110, as shown in FIG. 22A, and may be rotatable and slidable therein. Actuator shaft 1140 may be connected directly or indirectly to a proximal end of actuator rod 170. In this regard, actuator shaft 1140 may include a connection feature for connecting to actuator rod 170 in a secure manner such that it prevents relative rotation between actuator shaft 1140 and actuator rod 170. For example, actuator shaft 1140 may have a central opening that receives the proximal end of actuator rod 170 and a collet 1142 disposed within the central opening. Such collet 1142 may be configured to grasp or otherwise engage and secure actuator rod 170 to actuator shaft 1140. For example, collet 1142 may be threaded to actuator shaft 1140 and have fingers 1143 that are cammed inwardly by sloping or tapered inner surfaces 1141 of actuator shaft 1140 as collet 1142 is advanced distally within the central opening. Alternative connection features are also contemplated, such as a crimp ring and direct threaded engagement between actuator shaft and actuator rod, for example.

Actuator shaft 1140 may also have a proximal portion 1145, a distal portion 1149, and an intermediate portion 1147. In some examples, an actuator rod handle 1160 may be connected to proximal portion 1145 to help facilitate manual manipulation of actuator rod 170 when desired to release fixation device 112. In this regard, proximal portion 1145 may be positioned at least partially outside of axial channel 1114 of slider 1110. On the other hand, intermediate portion 1147 may be at least partially disposed within channel 1114. Intermediate portion 1147 may have a larger cross-sectional dimension than proximal and distal portions 1145, 1149 and may include a notch 1144 extending radially inwardly. Such notch 1144 may be configured to align with transverse opening 1111 of slider 1110, as shown in FIG. 22A, so that a deployment pin 1172 (FIGS. 22B-22D) extending through transverse opening 1111 and notch 1144 prevents actuator shaft 1140 from rotating and translating relative to slider 1110, as discussed in more detail further below.

In various examples, a bearing 1150 may be disposed within axial channel 1114 of slider 1110, and distal portion 1149 of actuator shaft 1140 may be removably received within bearing 1150, as shown in FIG. 22A. Bearing 1150 may be secured to slider 1110 via a press-fit or threaded connection, for example. Bearing 1150 may be a one-way bearing such that, when distal portion 1149 of actuator shaft 1140 is received within bearing 1150, actuator shaft 1140 is rotatable in a first direction but prevented from rotation in a second direction, for example. As mentioned above, in one example, actuator rod 170 may be released from fixation device 112 by rotating actuator rod 170 in the first direction which may unthread a distal end of actuator rod 170 from stud 131 or base 139 of fixation device 112. Thus, in some examples, bearing 1150 may allow actuator rod 170 to be rotated for release but prevent rotation in the opposite direction which may help prevent inadvertent overtightening. An exemplary positioner assembly with one-way bearing is disclosed in U.S. Pat. No. 10,660,625, the disclosure of which is hereby incorporated by reference herein in its entirety.

Actuator shaft 1140 may be coupled to slider 1110 via a deployment system 1170, for example. An exemplary deployment system 1170 is depicted in FIGS. 22A-22D and may include one or more of transverse opening 1111 of slider 1110, notch 1144 of actuator shaft 1140, and a deployment pin 1172 which may be releasable received within transverse opening 1111 and notch 1144. Deployment system 1170 may also include a handle 1170. Handle 1180 may connect to the deployment pin 1172 via a hinge 1183 in one example. In one example, handle 1180 may be pivotable relative to slider 1110 when pin 1172 is disposed therein so as to pivot between a locked configuration (shown in FIG. 22B) and an unlocked configuration (shown in FIGS. 22C and 22D). In the depicted embodiment, handle 1180 may extend around a portion of slider 1110 (e.g., about substantially one half of the slider 1110 or more). Handle 1180 may, for example, comprise a curved profile 1182 that corresponds to a curved profile 1116 of slider 1110, which advantageously minimizes a profile of the deployment system 1170 in the engaged configuration. This permits removal of pin 1172 when handle 1180 is rotated to the unlocked configuration and prevents removal of pin 1172 when handle 1180 is in the locked configuration. Handle 1180 may, for example, include at least one protuberance 1181 extending from an interior surface 1182 of handle 1180 which may cooperate with indentation or notch 1113 in slider 1110. Protuberance 1181 and notch 1113 can prevent handle 1180 from inadvertently rotating off of slider 1110. Handle 1180 may also include a pin cover 1184 in one example. Pin cover 1184 may be configured to cover and retain a pin tip 1174 when handle 1180 is in the locked configuration, which can in turn retain pin 1172 within slider 1110, as shown in the example of FIG. 22B. Pin cover 1184 may have a groove 1188 which can provide a space for the pin tip 1174. When provided, such covering 1184 helps prevent inadvertent contact with pin 1172 that could result in premature disengagement. A spring biased ball 1179 may also be included in pin 1172 to help further secure pin 1172 from being inadvertently removed from slider 1110 in some examples.

In operation, positioner assembly 1100 may be utilized to move distal elements 120 between the open, closed, and inverted positions. In one example, rotating actuator knob 1120 in the first direction may translate slider 1110 and consequently actuator rod 170 proximally which may move distal elements 120 from the closed to open position and from the open position to the inverted position. On the other hand, rotating actuator knob 1120 in the second direction may advance slider 1110 and consequently actuator rod 170 distally which may move distal elements 120 from the inverted position to the open position and from the open position to the closed position. When it has been determined that the valve leaflets have been sufficiently grasped by fixation device 112, fixation device 112 may be released from delivery catheter 1020. In one example, this may be achieved by releasing handle 1180 from slider 1110 and removing pin 1172 from slider 1110 which frees actuator shaft 1140 from slider 1110. Once pin 1172 has been removed, actuator shaft 1140 and actuator rod 170 may be rotated via actuator handle 1160 in the first direction thereof to thereby release actuator rod 170 from stud 131 or base 139. Once released, actuator rod 170 can be pulled proximally relative to slider 1110 which retracts actuator rod 170 and positions it proximal to the coupling interface between shaft 111 and coupling member 160, as described above with respect to coupling system 115 of FIGS. 7A and 7B and also similarly described with respect to coupling system 315 of FIGS. 8A and 8B. Other exemplary deployment control systems and positioner assemblies that may be used in delivery system 1000 are disclosed in U.S. Pub. No. 2021/0353419 (“the '419 Publication”), the disclosure of which is hereby incorporated by reference herein in its entirety.

FIGS. 21B and 23A-23C depict gripper control assembly 1200 according to an embodiment of the present disclosure. Examples of the gripper control assembly 1200 may generally include one or more of a first element line handle 1202a, a second element line handle 1202b, and an interlock 1210 selectively coupling first and second element line handles 1202a, 120b to each other. In one example, first proximal element line 101a may be coupled to first gripper element line handle 1202a, and second proximal element line 102b may be coupled to second gripper element line handle 1202b. Each of these handles 1202a, 1202b may separately and independently actuate first and second proximal element lines 101a, 101b so as to separately and independently raise and lower proximal elements 140 between their raised and lowered positions, according to examples of the disclosure. However, when interlock 1210 couples first and second element line handles 1202a, 1202b together, first and second proximal element lines 101a, 101b and, consequently, proximal elements 140 may be actuated concurrently in some examples. In the embodiment depicted, first and second proximal element line handles 1202a, 1202b can be aligned in parallel which helps facilitate concurrent actuation. Although two proximal element line handles 1202a, 1202b are depicted, one proximal element line handle may be provided where a single proximal element line 101 is coupled to both proximal elements 140, such as in the interventional device 110′ arrangement depicted in FIG. 15. It should also be understood that a third proximal element line handle can also be provided where fixation device 112 may include a third proximal element 140 in some examples.

First and second proximal element line handles 1202a, 1202b may each include a connection mechanism for connecting to respective proximal element lines 101a, 101b. In the embodiment depicted, each element line handle 1202a, 1202b may connect to a proximal end portion 103a (see FIG. 23C) of its respective proximal element line 101a, 101b, while a second end portion 103b of each proximal element line 101a, 101b may be releasably coupled to shaft 111, such as described with respect to FIGS. 16 and 17A-17F. Thus, in the embodiment depicted, the connection mechanism of each proximal element line handle 1202a, 1202b may be configured to connect to first end portion 103a of a respective proximal element line 101a, 101b. For example, the connection mechanism can include a collet 1220 that may securely grasp proximal end portion 103a of proximal element lines 101a, 101b. As shown, first proximal element line handle 1202a may have a lumen 1203 with a central axis, and a screw 1224 therein. Screw 1224 can have a lumen 1223 with collet 1220 therein, collet 1220 may have fingers 1222, for example. First end 93a of first proximal element line 101a can be disposed within with a recess defined by fingers 1222 according to one example. When screw 1224 is tightened, collet fingers 1222 may tighten down on and grasp proximal element line 101a. In this regard, proximal element line 101a may be fixed in a tensioned or non-tensioned state. Proximal element line 101b may be coupled to proximal element line handle 1202b using a similar collet system (i.e., connection mechanism). Although the depicted embodiment includes a connection mechanism including a collet 1220, it should be understood that other connection mechanisms may be utilized for securing a first end 93a of a proximal element line 101a, 101b to a proximal element line handle 1202a, 1202b, such as a swage or a knot, for example.

One or more of proximal element line handles 1202a and 1202b may also include respective end caps 1206a, 1206b disposed at their proximal ends in some examples. Each of the end caps 1202a and 1206b can, for example, include finger grip portions, such as one or more top concave portions 1207. In various examples, one or more of end caps 1206a and 1206b may include a bump 1208 which may provide tactile feedback to the surgeon as to which handle 1206a, 1206b and corresponding proximal element 140 is being actuated. Other tactile features can be utilized such as protrusion, ridge, or roughened surface, for example.

Interlock 1210 may, for example, be moveable between an unlock position in which the first and second proximal element line handles 1202a and 1202b are independently actuatable and a locked position in which first and second proximal element line handles 1202a and 1202b are coupled together to be actuatable together. Interlock 1210 can be fixedly or moveably coupled to first proximal element line handle 1202a and releasably coupled to second proximal element line handle 1202b. Interlock 1210 can include, for example, a latch 1212 and recess 1213. Latch recess 1213 can receive latch 1212, such that proximal element line handles 1202a and 1202b are operably connected for concurrent actuation. In one example of the unlocked position, latch 1213 can be removed from recess 1213, such that the proximal element line handles 1202a and 1202b are not operatively connected and are independently actuatable. Latch 1213 can be configured to be moved linearly, rotationally, or in any suitable motion to lock and unlock the interlock 1210 as described. Also, as shown in FIG. 23B, latch 1213 and recess 1213 can have complementary dovetail shapes in various examples. The dovetail shapes can limit the proximal element line handles 1202a and 1202b from pulling away from one another when the latch 1212 is engaged with recess 1213. The dovetail shapes can also prevent interlock from switching between the lock and unlock positions. A locking lever 1214 can be provided to facilitate movement of latch 1212 in some examples. Actuating interlock 1210 between the locked position and the unlocked position can, for example, be performed with a single hand or a single finger. Either of latch 1212 and recess 1213 can be disposed in either of first proximal element line handle 1202a and second proximal element line handle 1202b. Other interlock examples that may be used in gripper control assembly 1200 are disclosed in U.S. Pub. No. 2021/0015614, the disclosure of which is hereby incorporated by reference herein in its entirety.

Proximal element line handles 1202a, 1202b may each be moveably coupled to a base 1201 which may be secured to housing 1012 of delivery system handle 1010 in one example. Base 1201 provides structural support for gripper control assembly 1200 and helps maintain proper alignment of each proximal element line handle 1202a, 1202b during actuation. In this regard, each proximal element line handle 1202a, 1202b and their respective connection mechanisms may be moveable in a proximal-distal direction between a first position and a second position relative to base 1201, according to one example. The movement between positions helps provide consistent tensioning of the proximal element lines 101a, 101b and corresponding movement of proximal elements 140 regardless of variations in operator technique. One example of a first position is shown in FIG. 21B and is a tensioned position in which proximal element lines 101a, 101b are tensioned so that proximal elements 140 are in their raised position. Pushing proximal element line handles 1202a, 1202b in a distal direction moves handles to their second position (see FIG. 23C) which releases the tension and correspondingly lowers proximal elements 140. Base 1201 and proximal element line handles 1202a, 1202b may together form a ball-detent mechanism or a snap-fit mechanism, for example, which may releasably and respectively secure proximal element line handles 1202a, 1202b in the first and second position. The detent mechanism provides tactile feedback to the operator confirming that the handles 1202a, 1202b have reached their respective positions and helps prevent unintended movement during the procedure. As illustrated in FIG. 21B, base is disposed within housing 1012 such that, when proximal element line handles 1202a, 1202b are in an example of their second position, a majority of their respective lengths are disposed within base 1201 and handle housing 1012. This helps secure gripper control assembly 1200 and provide gripper control assembly 1200 a low profile.

A first sheath 1220a and second sheath 1220b may extend from a distal end of base 1201 and to a manifold 1320 of fluid management assembly 1300, as described in more detail below. In one example, first proximal element line 101a may extend through first sheath 1220a, and second proximal element line 101b may extend through second sheath 1220b. Sheaths 1220a, 1220b may help shield proximal element lines 101a, 101b from rubbing or snagging on other components within handle 1010 as they are actuated. Additionally, sheaths 1220a and 1220b help prevent fluid from escaping fluid management assembly 1300 and or contaminants from entering fluid management assembly 1300.

As shown in FIGS. 21B, 23A, and 24C, fluid management assembly 1300 may generally include a fluid chamber 1310, a connector 1330, and a manifold 1340 separating chamber 1310 from connector 1330 according to various examples of the disclosure. Fluid chamber 1310 may also be a lock handle housing, as described in more detail below. Fluid chamber 1310 may include a chamber volume 1318 and chamber lid 1314 covering chamber volume 1318 in one example. In one example, an O-ring 1313 seals lid 1314 to fluid chamber 1310, as best shown in FIG. 24C. Chamber lid 1314 may include a first inlet/outlet port 1302a which may define a luer lock for connection to a flush bag or the like for delivering/receiving fluid to or removing air from chamber 1310 in some examples. Chamber 1310 may also include a lock handle extension or cylindrical extension 1350 extending from a side thereof according to various examples. Such lock handle extension 1350 may be configured to receive a lock line knob, as described further below. Additionally, fluid chamber 1310 may include a first opening 1316 at proximal end and a second opening 1317 at a distal end, as shown in the example of FIG. 24C.

Connector or fluid inlet block 1330 may be disposed distal of fluid chamber 1310 and may be configured to connect to a proximal end portion 1021 of delivery catheter 1020, such as over at least a portion of proximal end portion 1021 of delivery catheter 1020, in some examples. In this regard, proximal end portion 1021 of delivery catheter 1020 may be connected to connector 1330 such that fluid may flow between connector 1330 and delivery catheter 1020 lumens to lubricate lines 101a, 101b, 102 and shaft 170 and over an exterior of delivery catheter 1020 to ensure smooth translation of delivery catheter relative to sleeve 1030, in some examples. Connector 1330 may also include a second inlet/outlet port 1302b for fluid transfer, for example. Second port 1302b, however, may extend from an opposite side of handle 1010 from first inlet/outlet port 1302a in various examples.

Manifold 1340 may be positioned between fluid chamber 1310 and connector 1330 and may be connected to a proximal end of connector 1330, as shown in the example of FIGS. 21B and 23A. Manifold 1340 may include a shaft 1342 extending proximally therefrom which may be coupled to second opening 1317 of fluid chamber 1310 in one example. Shaft 1342 may have a plurality of lumens extending therethrough each for receipt of any one of actuator rod 170, a first end portion 102a of lock line 102, and a second end portion 102b of lock line 102 (see e.g., FIG. 24M). Second opening 1317 of fluid chamber 1310 may include an O-ring 1317a and ring retainer 1317b to provide a leak free seal between fluid chamber 1310 and shaft 1342, for example. As illustrated in the example of FIG. 23A, actuator rod 170 may extend into first opening 1316 of fluid chamber 1310 and may pass through fluid chamber 1310 into shaft 1342 of manifold 1340 where it may then be directed into delivery catheter 1020. First opening 1316 of fluid chamber 1310 may include a mandrel seal 1316a and mandrel retainer 1316b (see FIG. 24C) for sealing the interface between actuator rod 170 and fluid chamber 1310 in various examples. Proximal element line sheaths 1220a, 1220b may be connected to manifold 1340 such that proximal element lines 101a, 101b extending through sheaths 1220a, 1220b are directed through manifold 1340 and into delivery catheter 1020 according to various examples.

In one example, fluid may enter fluid management system 1300 via port 1302b and into connector 1330. Fluid from connector 1330 may then travel distally into the lumens of delivery catheter 1020 to lubricate the control elements therein, such as gripper lines 101a and 101b, and lock line end portions 102a and 102b, for example. Additionally, fluid from connector 1330 may flow distally between delivery catheter 1020 and sleeve 1330 to lubricate relative translation, as described in more detail below. Fluid may also travel proximally from connector 1330 through manifold 1340 and into sheaths 1220a, 1220b to lubricate gripper lines 101a, 101b and through lock line lumens within shaft 1342 and into fluid chamber 1310. Once fluid is located within chamber 1310, it may then travel distally through a lumen for actuator rod 170 which may pass through shaft 1342 and into delivery catheter 1020. Inlet/outlet port 1302a may be used to deair the system.

The arrangement of fluid management assembly 1300, as described above and shown in the figures, significantly reduces the volume of fluid delivered to and from delivery system handle 1310 as compared to prior delivery systems and provides a single point flush and anchoring to handle 1310 while ensuring fluid runs within delivery catheter 1020 lumens and over an outer surface of delivery catheter 1020. Such reduction may be upwards of 3.7 times that of prior delivery systems and results in a more efficient deairing of system 3 with a reduction in the amount of flush bags utilized. This efficiency is particularly beneficial during time-sensitive cardiac procedures where rapid and complete deairing is essential to prevent air embolism. Fluid volume of fluid management assembly 1300 may be about 14 cubic centimeters, for example, which represents an optimal balance between adequate fluid delivery for lubrication and minimal volume for efficient deairing. The reduced fluid volume also decreases the system's overall weight enhancing operator handling and control during delicate procedures. Fluid volume of fluid management assembly 1300 may be about 14 cubic centimeters, for example. Such reduction in volume is facilitated at least by the knob configuration of lock line assembly 1400, which is described in more detail below. Fluid management assembly 1300 also decreases overall preparation time and decreases the risk that a flush line may become tangled at least due to a reduced number of flush line connections (e.g., one total flush line connection to port 1302b). The simplified connection arrangement enables faster setup and reduces the potential for operator error. Furthermore, the arrangement of fluid management assembly 1300 reduces the number of hemostatic valves that may be needed which correspondingly increases the torque transmission ratio from delivery catheter handle 1010 to fixation device 112, resulting in more responsive and precise control during valve repair procedures.

FIGS. 24A-24O depict lock control assembly 1400 according to an embodiment of the present disclosure. Lock control assembly 1400 may actuate locking mechanism 116 or 516 between its locked and unlocked configuration, for example. Lock control assembly 1400 may, for example, also be configured to release lock line 102 from fixation device 112. Lock control assembly 1400 may generally include one or more of a lock line handle 1402, a lock line handle housing 1310, a spool 1460, and a locking system 1404.

Referring to the example of FIG. 24C, lock line handle 1402 may generally include a lock knob 1410 and a lock handle shaft 1440 extending from lock knob 1410.

In one example, lock knob 1410 may be configured to be gripped by a user and pivoted relative delivery system handle 1010 to operate lock line 102 (as described in greater detail below). Lock knob 1410 can have a pear shape which can act as a flag or pointer to help indicate when lock knob 1410 is in a locked or unlocked position. Although shown as a pear shape, lock knob 1410 can have any suitable shape and/or configuration. Also, lock knob 1410 may have a recess 1413 which may also be pear shaped. However, other shapes are contemplated such as rectangular or oval, for example. Additionally, lock knob 1410 may have an opening 1411 (e.g., a cylindrical shaped opening) extending therethrough and intersecting recess 1413.

In the embodiment depicted, lock knob 1410 may include a lock knob insert 1420 and a lock knob cap 1412. Lock knob insert 1420 may include an insert body 1422 that may be cylindrical and a flange 1421 that may be connected to one end of insert body 1422. Flange 1421 may have the same (i.e., corresponding) shape as lock knob recess 1413 in various examples. Thus, where lock knob recess 1413 is pear shaped, flange 1421 may have a pear shape. Insert body 1422 may have an inner surface that may define an opening or passageway 1425 that may extend entirely through insert body 1422 in some examples. Inner surface of insert body 1422 may include a plurality of teeth or splines that may be configured to enmesh with corresponding teeth or splines 1454 on lock handle shaft 1440 (see e.g., FIG. 24F), as discussed further below. Insert body 1422 may also include a channel 1424 extending circumferentially about an exterior of insert body 1422 and may include a plurality of detents 1423 which may each extend radially inwardly and, in one example, may intersect channel 1424, for example. In various assembled examples, flange 1421 may be disposed within recess 1413 of lock knob 1410 while insert body 1422 may extend through cylindrical opening 1411 of lock knob 1410. The shape (e.g., pear) of lock knob recess 1413 and corresponding shape (e.g., pear) of flange 1421 can limit relative rotation between lock knob insert 1420 and lock knob 1410. Lock knob cap 1412 may be connected to lock knob 1410, such as via a snap-fit connection and/or threaded connection, for example. Lock knob cap 1412 may be positioned over flange 1421 of insert 1420 which may sandwich flange 1421 between lock knob 1410 and lock knob cap 1412 thereby retaining lock insert 1420. Lock knob cap 1412 may also have an opening or a recess 1412a that may align with the opening 1425 of lock knob insert 1420, as shown in the example of FIG. 24C, and may provide clearance for a lever arm 1442 of lock handle shaft 1420 to be partially positioned therein in a locked position. Although lock knob 1410, lock knob insert 1420, and lock knob cap 1412 are shown and described as being separate components, it should be understood that each of these components can be integrated together so as to form a monolithic lock knob structure, such as by an injection molding process or additive manufacturing process, for example.

FIGS. 24D-24H depict one example of lock handle shaft 1440. Lock handle shaft 1440 may generally include one or more of a shaft body 1450 and a lever arm 1442 pivotably connected to shaft body 1450. Shaft body 1450 may include a first end portion 1452 and a second end portion 1453. An opening 1451 may extend through the length of shaft body 1450 from first end portion 1453 to second end portion 1453. As shown in the example of FIGS. 24E and 24F, opening 1451 may be larger at first end portion 1452 than at second end portion 1453. First end portion 1452 of shaft body 1450 may be substantially cylindrical and may be configured to be received within a corresponding opening 1465 in spool 1460 (see e.g., FIG. 24B). A notch 1456 may be formed in first end portion 1452, and a fin or tab 1455 may extend radially outwardly from first end portion 1452 in some examples. Fin 1455 may be configured to be received within a corresponding notch 1462 in spool 1460 (see FIG. 24C), for example. In the embodiment depicted, fin 1455 and notch 1462 may be circumferentially aligned. Lock handle shaft body 1450 may, for example, also include an O-ring 1459 positioned between first and second end portions 1452, 1453 for forming a seal between lock handle shaft 1440 and lock handle extension 1350 of lock handle housing 1310 when received therein. Additionally, shaft body 1450 may include a plurality of teeth or splines 1454 which may enmesh with the teeth of lock knob insert 1420 which may rotationally constrain lock handle shaft 1440 relative to lock knob insert 1420 when disposed therein according to various examples of the disclosure. As shown in the example of FIGS. 24F and 24G, second end portion 1453 may include a post opening or hinge opening 1453a for a corresponding post 1444 of lever arm 1442. Also, second end portion 1453 may include an abutment feature 1457 extending radially outwardly therefrom which may be configured to constrain a range of motion of lever arm 1442 when coupled to body 1450, for example.

In various examples, lever arm 1442 of lock handle shaft 1440 may include a lever body 1445 and post or hinge 1444 extending from lever body 1445. Post 1444 may be rotatably received within post opening 1453a of shaft body 1450 in some examples. Lever arm 1442 may also include a first opening 1441a that passes through lever body 1442 and a second opening 1441b that passes through post 1444 (see the example of FIG. 24H). However, in some embodiments, lever arm may only include opening 1441b passing through post. First and second openings 1441a, 1441b may be axially aligned in various examples. As shown in the example of FIG. 24N, lock line 102 may include a first end portion 102a and a second end portion 102b. First end portion 102a may be fixedly secured to shaft body 1450, such as to an inner surface thereof. Prior to removal of lock line 102 from fixation device 112, second end portion 102b of lock line 102 may extend through opening 1451 in shaft body 1450, and first and second openings 1441a, 1441b of lever arm 1445. In one example, lever arm 1445 may be rotatable about an axis of post 1444 and relative to shaft body 1442 between a first position or unlocked configuration (see FIGS. 24D, 24E, and 24N) and second position or locked configuration (see FIGS. 24F and 24G). In one example of the unlocked configuration, lever arm 1442 may be axially aligned with shaft body 1445 such that first and second openings 1441a, 1441b of lever arm 1442 are coaxially aligned with opening 1451 of shaft body 1450. This may allow second end portion 102b of lock line 102 to be passed through lock handle shaft 1440 as it is pulled away from delivery system handle 1010, as best shown in FIG. 24N. In one example of the locked configuration, lever arm 1442 may be rotated such that it is angled relative to shaft body 1450. In this regard, second opening 1441b within post 1444 may be angled relative to opening 1451 in shaft body 1450 such that a segment of lock line 102 is trapped between post 1444 and shaft body 1450. For example, lever body 1445 may be rotated 90 degrees relative to shaft body so that lock line 102 may be trapped and compressed along an arc subtending an angle of about 90 degrees and second opening 1441b is oriented orthogonal to opening 1451. However, in other embodiments, lever body may be rotated to angles less than or greater than 90 degrees, such as 45 degrees to 135 degrees, for example. This may secure second end portion 102b of lock line 102 to lock handle shaft 1440 to prevent it from being decoupled from fixation device 112 until desired. Although it has been described that shaft body 1450 includes a female post opening and lever arm 1442 includes a male post, it is also contemplated that shaft body 1450 may include the male component, while lever arm may include the female component. In other words, shaft body 1450 may include a post, like that of post 1444, with a lock line opening extending therethrough, while lever arm 1442 may include a post opening, like that of opening 1444, and a lock line opening, such as opening 1441a, intersecting such post hole. Lever arm 1442 may be provided with a readily identifiable color as compared to other features of lock control assembly 1400 so as to identify lever arm 1442 with fixation device deployment in some examples. Such color may be magenta, for example.

As illustrated in the example of FIGS. 24C and 24N, lock handle shaft 1440 may extend through lock knob insert 1420 and lock knob 1410 so that lever arm 1442 extends from lock knob 1410 and first end portion 1452 of shaft body 1450 extends from insert 1420 so that it can be received with spool 1460. Teeth 1454 of shaft body 1450 may mesh with teeth of insert 1420 so as to rotationally constrain shaft body 1450 relative to insert 1420 and allow them to rotate together in some examples. Lock handle shaft 1440 may be secured to lock knob 1410 via a hairpin clip 1430 which may be inserted between knob 1410 and knob cap 1412 and engage a circumferential groove 1458 in shaft body 1450 according to various examples. In this arrangement, lever arm 1442 may be moved between the locked and unlocked configuration to correspondingly secure and release second end portion 102b of lock line 102. Abutment feature 1457 may limit rotation of lever arm 1442 so that when abutment feature 1457 stops the rotation of lever arm 1442, it is in the unlocked configuration, according to various examples of the disclosure.

Spool 1460 may have a shaft opening 1465 for receipt of first end portion 1452 of lock handle shaft 1450, as shown in the example of FIG. 24B. Additionally, spool 1460 may include a notch 1462 which may be configured to receive fin 1455 of shaft body 1450 and a first lock line opening 1464a which may be aligned with notch 1462 and may extend through a track 1466 of spool 1460, as best shown in the example of FIG. 24C. This may allow first end portion 102a of lock line 102 to pass through notch 1456 in shaft body 1450 and through first lock line opening 1464a so that it can be partially wrapped around spool 1460 and extend through opening 1317 in housing 1310 to delivery catheter 1020, as shown in FIG. 24B. In one example, spool 1460 may include a second lock line opening 1464b which may extend through track 1466 of spool 1460 and through a side of spool 1460 which may allow second end portion 102b of lock line 102 returning from delivery catheter 1020 to extend through second lock line opening 1464b out of spool 1460 and into opening 1451 of lock handle shaft 1440, as shown in FIG. 24B.

As mentioned above, lock handle housing 1310 may also be fluid chamber of fluid management assembly 1300. In some examples, spool 1460 may be disposed within internal volume 1318 of lock handle housing 1310 such that it is aligned with cylindrical extension 1350 extending therefrom, as shown in FIG. 24B. As mentioned above, spool 1460 can be rotationally fixed relative lock handle shaft 1440, for example, by a keying feature, such as fin 1455 and notch 1462. Accordingly, rotation of lock knob 1410, which can cause rotation of lock handle shaft 1440, can thereby cause rotation of spool 1460 and therefore increase or decrease tensile load on lock line 102 by way of lock line 102 spooling or unspooling around spool 1460. Lock handle housing 1310 can include a dowel pin 1319a in one example. First end portion 102a of lock line 102 can be routed around dowel pin 1319a and fin 1319b in lock handle housing 1310. This can align first end portion 102a of lock line 102 with track 1466 of spool 1460 and can manage slack in first end portion 102a of lock line 102. If slack is not managed, first end portion 102a of lock line 102 can disengage from track 1466 and from spool 1460. As noted above, first end portion 102a and second end portion 102b of lock line 102 may extend through opening 1317 in housing 1310 so as to extend to and from delivery catheter 1020, respectively. In one example, first end portion 102a can be routed around dowel pin 1319a and relative fin 1319b and routed at least partially around spool 1460 along track 1466 to first lock line opening 1464a where it passes through spool and enters opening 1451 of lock handle shaft 1140 via notch 1456. First end portion 102a may be fixedly secured to shaft body 1450 in one example. Additionally, in some examples, second end portion 102b may be routed around dowel pin 1319a, through second lock line opening 1464b in spool 1460, and through opening 1451 in lock handle shaft 1440 where it may be releasably secured via lever arm 1442.

Delivery system 1000 can be flushed, for example, via port 1302a in cover 1314 of lock handle housing 1310. For example, flush fluid can enter through port 1302a into the lock handle housing 1310 and out through opening 1317 into connector 1330. During preparation of system 1000, air can exit delivery system handle 1010 through second port 1302b of connector 1330 in some examples.

In one example, lock handle extension 1350 may rotatably receive lock handle shaft 1440 of the lock line handle 1402 and cylindrical body 1422 of lock knob insert 1420. As mentioned above, lock control assembly 1400 may include a locking system 1404 to limit movement of lock line handle 1402 relative delivery system handle 1010 and removal of lock line handle 1402 relative handle 1010. Lock system 1404 can be a latch-detent lock in one illustrative example. For example, the latch can include a distal U-lock 1470 and a proximal U-lock 1480 coupled to one another (and collectively referred to as the “U-lock” or “lock body”) and configured to surround lock handle extension 1350, as shown in FIGS. 24B and 24C. Proximal U-lock or first lock member 1480 can include a pin 1482 which can engage detents 1423 on lock knob insert 1420, when pin 1482 is insert through window 1352 disposed in lock handle extension 1350. In addition, as mentioned above, channel 1424 may intersect detents 1423. Thus, pin 1482 may also engage channel 1424 when pin 1482 is not engaging detents. This helps prevent lock knob insert 1420 and the rest of lock handle 1402 from being inadvertently removed when rotating handle 1420 between the different positions (i.e., locked, unlocked, and third position described below). Distal U-lock or second lock member 1470 can include a rail 1472 to engage channel 1424 on lock knob insert 1420 according to one example.

In one example, locking system 1404 can further include a delivery handle insert 1490 and a release slider 1494, as shown in FIG. 24C, which can be used to release lock knob 1410. Delivery handle insert 1490 can include an indication, for example, an icon (such as a lock) or writing, to indicate that the delivery handle insert 1490 and release slider 1494 are intended to lock the lock line handle 1402. For example, and not by way of limitation, release slider 1494 can include a pin 1496 that can engage proximal U-lock 1480 (for example, through a window 1491 in delivery handle insert 1490). A spring 1484 or other biasing mechanism can be supported on pin 1354 of lock handle extension 1350 and can bias the latch into the locked position, for example. That is, spring 1484 can bias the latch in a distal direction to cause pin 1482 to engage detents 1423. Engagement of pin 1482 with detents 1423 can provide feedback, for example, audible and/or tactile, for a user that lock knob 1410 has reached a specific position (e.g., lock, unlock, override (also referred to herein as the third position or troubleshooting position)). Pin 1482 and detents 1423 can be arranged such that lock knob 1410 will not move from a specific position (e.g., lock, unlock, override), however, a user can move lock knob 1410 (i.e., overcome the engagement between the pin 1482 and the detents 1423) by turning knob 1410, and without touching release slider 1494. A portion of distal U-lock 1470, for example, rail 1472 on distal U-lock 1470 and channel 1424 can be arranged such that lock line handle 1402 can be removed from delivery handle 1010 when distal U-lock 1470 is disengaged from channel 1424 of knob insert 1420. For example, a user can slide release slider 1494 to disengage distal U-lock 1470 from channel 1424 and thereby release lock line handle 1402. For example, sliding release slider 1494 in the proximal direction can disengage rail 1472 on distal U-lock 1470 from channel 1424 and thereby permit the user to pull lock line handle 1402 from delivery handle 1010.

In an exemplary operation, fixation device 112 can be delivered in a locked position (or first position). Lock knob 1410 can be in the locked position, which is shown for example, in FIGS. 21A and 24I. In the locked position, lock line 102 can have no (or relatively low) tension and lock knob 1410 can point distally. Spring 1484 can bias distal U-lock 1470 and proximal U-lock 1480 in a distal direction and therefore bias pin 1482 to engage detent 1423 of lock knob insert 1420 (FIG. 24I).

During one example of a procedure, the user can turn the lock knob 1410 to the unlocked position to apply tension (or relatively higher tension) to lock line 102. Turning lock knob 1410 can overcome the engagement of pin 1482 with detent 1423. For example, lock knob 1410 can be rotated in a clockwise rotation (as viewed in FIG. 21A) such that lock knob 1410 can point upwardly (FIG. 24J). In such a position, lock knob 1410 itself can indicate to the user that fixation device 112 is in the unlocked position. Rotating lock knob 1410 can cause spool 1460 to rotate (as described above) and can increase tension on lock line 102 as lock line 102 spools around spool 1460. Such tensioning may tension both first and second portions 102a, 102b of lock line 102 so as to have an even pull on locking mechanism 116 or 516. As noted above, increasing tension on lock line 102 can apply a force on harness 340, 540 which can unlock fixation device 112, as described above. Spring 1484 can bias distal U-lock 1470 and proximal U-lock 1480 in a distal direction and therefore bias pin 1482 to engage detent 1423 of lock knob insert 1420 (FIG. 24J) and bias rail 1472 on distal U-lock 1470 to engage channel 1424 of lock knob insert 1420. Detents 1423 (as well as labeling) can define the locked and unlocked positions. A click sound can be made as pin 1482 engages detent 1423. Detent 1423 can also prevent lock knob 1410 from returning to the locked position (until a user engages lock knob 1410).

In one example, the user can lock fixation device 112 by rotating lock knob 1410 in the counterclockwise direction (as viewed in FIG. 21A) to return lock knob 1410 to the locked position, unspool lock line 102 from spool 1460, and decrease tension on lock line 102. A click sound can be made as pin 1482 engages detent 1423. Rail 1472 can remain engaged with channel 1424 even as handle 1402 moves between the locked, unlocked, and third position according to an example of the disclosure. In one example, lock knob 1410 can be rotated beyond the unlock position (see e.g., FIG. 24K), to a third position corresponding to a third detent 1423, and to thereby further increase tension on lock line 102. This can be useful during certain procedures to ensure that fixation device 112 properly locks. In this regard, the third position can be considered an override or troubleshooting position which may be used in the event the second position corresponding to second detent 1423 is not effective to unlock lock 116 or 516. In various examples, additional detents 1423 can be provided to engage pin 1482 and hold lock knob 1410 in the third position or further rotated positions. For example, a fourth position can correspond to a fourth detent 1423 and may provide an additional override position. Rail 1472 can remain engaged with channel 1424 even as knob 1410 moves between the locked, unlocked, and third positions. Although described in a particular arrangement, the third position, and any further positions, can include any suitable arrangement.

Before fixation device 112 can be deployed from delivery system 1000, lock line 102 may be decoupled from fixation device 112. To remove lock line 102, in one example, the user can ensure that lock knob 1410 is in the locked position. The user can also rotate lever arm 1442 of lock handle shaft 1440 to the unlocked position and retract release slider 1494 (i.e., the release slider 1494 can be pulled in the proximal direction). This can pull proximal U-lock 1480 (for example via pin 1496) and distal U-lock 1470 in a proximal direction (FIGS. 24L and 24M), which can disengage rail 1472 of distal U-lock 1470 from channel 1424. The user can pull lock line handle 1402 off delivery handle 1010 (FIG. 24N). Withdrawing lock line handle 1402 while lever arm 1442 is in the unlocked position can release second end portion 102b of lock line 102 from within lock handle shaft 1440, while first end portion 102a remains fixed to lock handle shaft 1440. Accordingly, in this example, withdrawing lock line handle 1402 pulls one end portion of lock line 102 (i.e., first end portion 102a), while the other end portion (i.e., second end portion 102b) is free to travel distally along the length of shaft 1440, through fixation device 112, and proximally along the length of delivery catheter 1020. Therefore, pulling lock line handle 1402 away from delivery handle 1010 withdraws lock line 102 from delivery system 1000 and fixation device 112.

If first end portion 102a of the lock line 102 becomes stuck, the user can disassemble lock line handle 1402 (FIG. 24O). For example, the user can remove hairpin clip 1430 and remove lock knob 1410 and lock knob insert 1420 from lock handle shaft 1440. This can provide direct access to first end portion 102a which is attached to lock handle shaft 1440. The user can pull second end portion 102b of lock line 102 to pull to free the “stuck” first end portion 102a. As described, lock control assembly 1100 is configured to provide discrete binary lock and unlock positions with tactile feedback which helps the clinician know when the locked and unlocked conditions are achieved. Additionally, lock control assembly 1100 with lock knob 1410 controls and limits the forces exerted on fixation device 112 thereby protecting its components from aggressive end user use and high lock line tension loads. The rotation of lock knob 1410 from the locked to unlocked position is configured to apply a sufficient tension to lock line 102, ensuring sufficient force to unlock fixation device 112 while preventing excessive tension that could damage components. The pear shape of lock knob 1410 serves as an ergonomic grip surface and as a visual indicator that allows operators to quickly verify the lock status from various viewing angles. When positioned in the distal direction, lock knob 1410 clearly indicates the locked position, while rotation to the upward direction provides immediate visual confirmation of the unlocked state. Other exemplary lock control assemblies that may be implemented in interventional system 5 are disclosed in U.S. Pat. No. 11,622,859, the disclosure of which is hereby incorporated by reference herein in its entirety.

Delivery catheter 1020 may include a proximal end portion 1021 and a distal end portion 1022, as best shown in FIG. 21B. As mentioned above proximal end portion 1021 of delivery catheter 1020 may be connected to delivery device handle 1010 and, in particular, may be coupled to connector 1330 of fluid delivery assembly 1300. Delivery catheter 1020 may include a plurality of lumens 1027 (see FIG. 16) extending from proximal end portion 1021 to distal end portion 1022. Each lumen may receive one of first proximal element line 101a, second proximal element line 101b, actuator rod 170, and proximal and distal portions of lock line 102a, 102b which each extend through distal end 1022 of delivery catheter 1020 and to fixation device 112 where they are respectively coupled to proximal elements 140, stud 131 or base 139, and locking mechanism 116 or 516. Distal end 1022 of delivery catheter 1020 may include shaft 111 extending distally therefrom for coupling to fixation device 112. As discussed above, actuator rod 170 may extend through shaft 111 and into fixation device 112 where it may be coupled to stud 131 or base 139. Distal end 1022 of delivery catheter 1020 may optionally include a nose 1024. In the embodiment depicted, nose 1024 has a flanged shape. Such a flanged shape prevents nose 1024 from being retracted into a guide catheter of steerable guide system 5. However, it may be appreciated that nose 1024 may have any shape including bullet, rounded, blunt or pointed, to name a few.

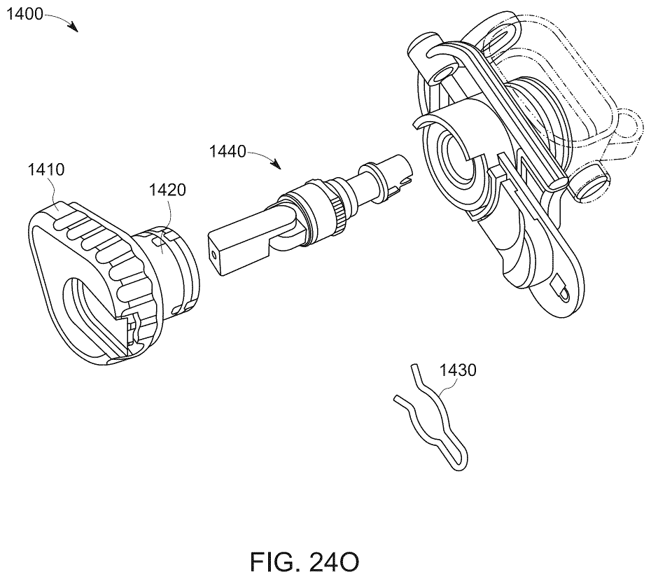

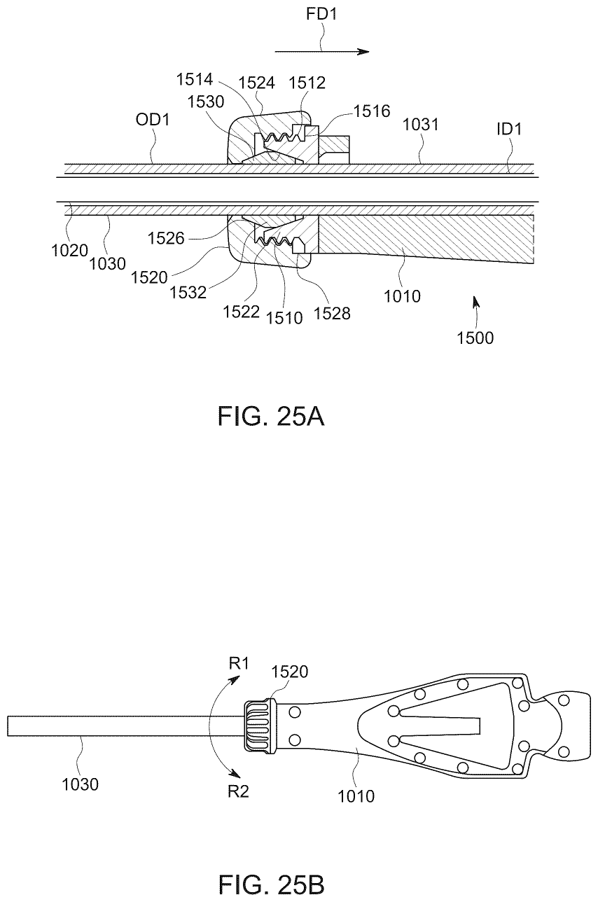

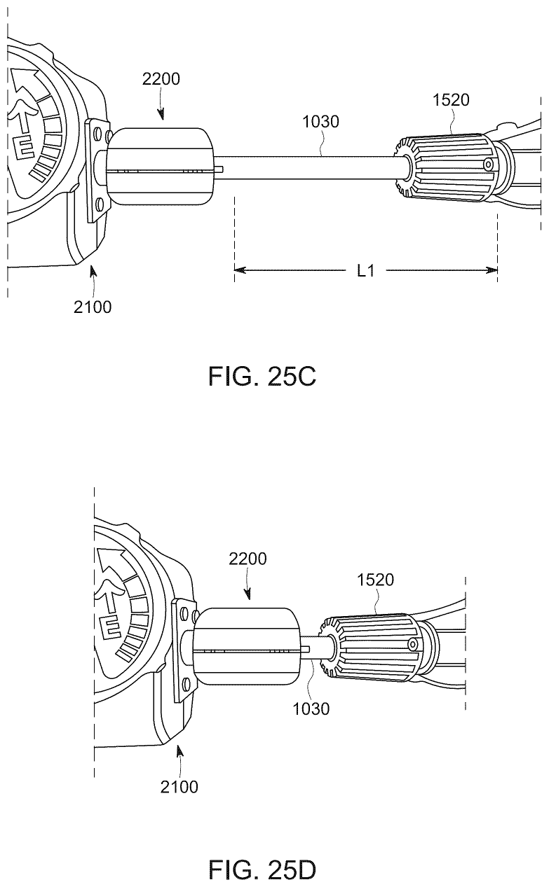

During a transcatheter procedure it may be desirable to advance and/or retract delivery catheter 1020 relative to steerable guide system 5 and to secure delivery catheter 1020 in a desired position relative to the target valve. FIGS. 21B and 25A-25D depict a delivery catheter fastening assembly 1500 according to an embodiment of the present disclosure and which may be configured to selectively secure delivery catheter 1020 from translation and release delivery catheter 1020 for proximal-distal translation. In the embodiment depicted, delivery catheter fastening assembly 1500 may generally include delivery catheter 1020, a brake shaft 1030, a distal end portion 1510 of delivery system handle 1010, a fastener knob 1520, and a compression ring 1530.

Brake shaft or sleeve 1030 may, in one example, have a first end portion or proximal end portion 1031 that that may extend through a distal opening of delivery system handle 1010 and may be slidable relative thereto, as best shown in FIGS. 21B and 25A. Brake shaft 1030 may also include a second end portion or distal end portion 1032 that may be fixedly secured to an inner guide catheter handle 2100 of steerable guide system 5, as shown in FIGS. 25C and 25D and as described in more detail below. Brake shaft 1030 may comprise an outer diameter OD1 and an inner diameter ID1. Delivery catheter 1020 may slidably and rotatably extend through brake shaft 1030 and inner diameter ID1 thereof, as shown in FIGS. 21B and 25A.

Distal end portion or static member 1510 may be disposed at a distal end of handle 1010 and may define the distal opening through which brake shaft 1030 and delivery catheter shaft 1020 extend. In this regard, proximal end portion 1031 of brake shaft 1030 may extend through distal end portion 1510 such that distal end portion 1510 circumferentially extends about outer diameter OD1, as shown in FIG. 25A. Distal end portion 1510 may include external threads 1512 and an inner engagement section 1514. Inner engagement section 1514 may be an inner sloping surface or chamfered portion that may taper radially inwardly toward brake shaft 1030 in a distal to proximal direction. Distal end portion 1510 may also have a stop surface 1516 disposed proximal of external threads 1512.

Fastener knob or dynamic member 1520 may be a threaded ring that may circumferentially extend about proximal end portion 1031 of brake shaft 1030 and may comprise internal threads 1522 that may be configured to cooperate or matingly engage with external threads 1512 of distal end portion 1510 of delivery system handle 1510, for example. Fastener knob 1520 may comprise an inner engagement section 1526 that may be an inner sloping surface or chamfered portion that tapers radially outwardly away from brake shaft 1030 in a distal to proximal direction. An outer surface 1524 of fastener knob 1520 may be tapered and/or may comprise textured or frictional features that may enhance grip. Additionally, fastener knob 1520 may have a stop-forming surface 1528 disposed proximal of internal threads and which may correspond to stop-forming surface 1516 of distal end portion 1510 which, when engaged, prevent further translation of fastener knob 1520 in a fastening direction FD1. This has the benefit of providing an indication to a practitioner that fastener knob 1520 has been adequately engaged with distal portion 1510 so as to reliably arrest translation of brake shaft 1030 and delivery catheter 1020.

Compression ring 1530 may circumferentially extend about outer diameter of brake shaft 1030 such that compression ring 1530 may come into direct contact with outer diameter OD1, as shown in the example of FIG. 25A. Compression ring 1530 may also be disposed between respective inner engagement sections 1514, 1526 of distal end portion 1510 and fastener knob 1520. Compression ring 1530 may comprise any suitable material including those with increased frictional properties. Such materials may include but are not limited to elastomeric or polymeric materials including medical-grade polymers, metal materials, ceramic materials, combinations thereof, or otherwise. Compression ring 1530 may define an outwardly bulging profile 1532 such that bulging profile 1532 generally peaks at a mid-section of compression ring 1530 and tapers inwardly towards its respective proximal and distal ends, according to one example. In this regard, with compression ring 1530 positioned between engagement sections 1514, 1526 of distal end portion 1510 and fastener knob 1520, inner sloping surfaces of distal end portion 1510 and fastener knob 1520 correspondingly engage bulging profile 1532, as illustrated in FIG. 25A.

During a transcatheter procedure it may be desirable to advance or retract delivery catheter 1030 to position fixation device 112 within a target valve. In one example, this may be achieved by unlocking fastening assembly 1500 by rotating fastener knob 1520 in a first rotation direction R1, as shown in FIG. 25B. The mechanical advantage provided by the threaded engagement between fastener knob 1520 and distal end portion 1510 allows the operator to easily overcome the compressive force on compression ring 1530 with minimal rotational effort. Rotating knob 1520 in direction R1 advances knob 1520 in a distal direction which relieves compression on compression ring 1530 and correspondingly friction on brake shaft 1030. The material composition of compression ring 1530 may include silicone, polyurethane, or other elastomeric compounds with specific durometer ratings selected to provide optimal compression characteristics and durability throughout repeated compression-decompression cycles. When the friction is sufficiently reduced, delivery system handle 1010 may be translated in a proximal or distal direction along brake shaft 1030 which correspondingly advances delivery catheter 1020 proximally or distally through brake shaft 1030, as illustrated in FIGS. 25C and 25D. The smooth internal surface of brake shaft 1030 may be coated with a low-friction material such as PTFE to further enhance the smooth translation of delivery catheter 1020. As handle 1010 is advanced distally, proximal end portion 1031 of brake shaft 1030 is moved further proximally within handle 1010. Conversely, as handle 1010 is moved proximally, proximal end portion 1031 of brake shaft 1030 moves toward distal end portion 1510 of fastening assembly 1500. As shown in FIG. 25C, the maximum travel length of handle 1010 relative to brake shaft is L1. This length L1 may be limited so as to prevent fixation device 112 from advancing too far and potentially damaging the heart. For example, in a transfemoral approach to a mitral valve or tricuspid valve, fixation device 112 may be initially positioned within an atrium and advanced toward the valve from the atrium. L1 may be limited so as to not overshoot valve and damage the chordae or ventricle. L1 may be about 3 to 4 inches, for example.

Once fixation device 112 is in a desired position relative to the target valve, fastening assembly 1500 may be locked by rotating fastener knob 1520 in a second rotation R2 direction opposite first rotation direction R1, as shown in the example of FIG. 25B. As fastener knob 1520 is rotated about distal portion 1510 of delivery handle 1010, fastener knob 1520 may translate in a longitudinal fastening direction FD1, which may be the proximal direction. As fastener knob 1520 is translated in the fastening direction FD1, engagement sections 1514, 1526 of distal end portion 1510 and fastener knob 1520 compress bulging profile 1532 of compression ring 1532 which correspondingly compresses compression ring 1532 against outer diameter OD1 of brake shaft 1030 until the friction resulting from such compression is sufficient to arrest proximal-distal translation of handle 1010 relative to brake shaft 1030. Stop-forming features 1516, 1528 of distal end portion 1510 and fastener knob 1520 may, for example, provide an indication that arrest has been achieved. By providing integrated fastening system 1500, superfluous floating components, which in practice are easily and frequently lost, are minimized, as fastener knob 1520 in cooperation with distal end portion 1510 of the delivery system handle 1010 provide a simplified, effective, and intuitive system for restricting or arresting translation of delivery catheter 1020 relative to brake shaft 1030 and correspondingly steerable guide system 5. Additionally, fastener knob 1520 improves ergonomics and ease of repeated and precise use by the practitioner by providing a conveniently sized and located component for effectively arresting translation. Other delivery catheter fastener examples that may be implemented in delivery system are disclosed in '419 Publication mentioned above and are incorporated herein by reference.

FIGS. 20, 26, 27A-27E, 28, and 29A-29B depict steerable guide system 5 according to an embodiment of the present disclosure. Steerable guide system 5 may be configured to guide delivery catheter 1020 and fixation device 112 coupled thereto percutaneously through the cardiovascular system to a position near the target valve. In the embodiment depicted, steerable guide system 5 generally includes an inner guide catheter assembly 2000 and an outer guide catheter assembly 3000. Inner guide catheter assembly 2000 may generally include one or more of an inner guide catheter handle 2100 and an inner guide catheter 2300 extending distally therefrom, and outer guide catheter assembly 3000 may generally include one or more of an outer guide catheter handle 3100 and outer guide catheter 3300 extending distally therefrom.

Outer guide catheter 3300 may have a proximal end 3301, a distal end 3302, and a central lumen extending therethrough, and inner guide catheter 2300 may have a proximal end 2301, a distal end 2302, and a central lumen extending therethrough. Inner guide catheter 2300 may be positioned coaxially within the central lumen of outer guide catheter 3300, as shown in FIG. 20. Distal ends 2302, 3302 of catheters 2300, 3300, respectively, may be sized to be passable to a body cavity, typically through a body lumen such as a vascular lumen. Outer guide catheter 3300 and/or the inner guide catheter 2300 may be precurved and/or have steering mechanisms to position distal ends 2302, 3302 in desired directions.

Steering of outer guide catheter 3300 and inner guide catheter 2300 may be achieved by actuation of one or more steering mechanisms. Actuation of the steering mechanisms may be achieved with the use of actuators which may be located on inner and outer guide catheter handles 2100, 3100. Outer guide catheter handle 3100 may be connected to proximal end 3301 of outer guide catheter 3300 and may remain outside of a patient's body during use. Outer guide catheter handle 3100 may include one or more steering actuators or steering knobs 3102 which may be used to bend, arc, or reshape outer guide catheter 3300, such as to form a primary curve. Inner guide catheter handle 2100 may be connected to proximal end 2301 of the inner guide catheter 2300 and may optionally join with outer guide catheter handle 3100 to form one larger handle. Inner guide catheter handle 2100 may include one or more steering actuator 2102 which may be used to bend, arc, or reshape inner guide catheter 2300, such as to form a secondary curve and move or sweep distal end 2302 of inner guide catheter 2300 through one or more angles of motion.

Steerable guide system 5 may have different configurations depending on the approach taken to the target valve and may differ depending on which valve is targeted. For example, approaching a tricuspid valve through the inferior vena cava may involve tighter turns than approaching a mitral valve from the inferior vena cava and across the interatrial septum. Thus, the steering mechanisms and actuators 2102, 3102 of those steering mechanisms for inner and outer guide catheter assemblies 2000, 3000 may be configured accordingly. Examples of inner and outer guide catheter assemblies, steering mechanisms, and actuators thereof which may be incorporated into interventional system 3 and utilized to target a mitral valve are disclosed in U.S. Pat. No. 7,226,467, the disclosure of which is hereby incorporated by reference herein in its entirety. Also, examples of inner and outer guide catheter assemblies, steering mechanisms, and actuators thereof that may be incorporated into interventional system 3 and utilized to target a tricuspid valve are disclosed in U.S. Pub. No. 2023/0131595, the disclosure of which is hereby incorporated by reference herein in its entirety.

During a transcatheter procedure, it may be desirable to support and stabilize steerable guide system 5 and delivery system 1000. However, it may also be desirable to move certain components thereof in a controlled manner. In this regard, interventional system 3 may include, in some examples, one or more of a proximal attachment assembly or first attachment assembly 2200 and a distal attachment assembly or second attachment assembly 3200.

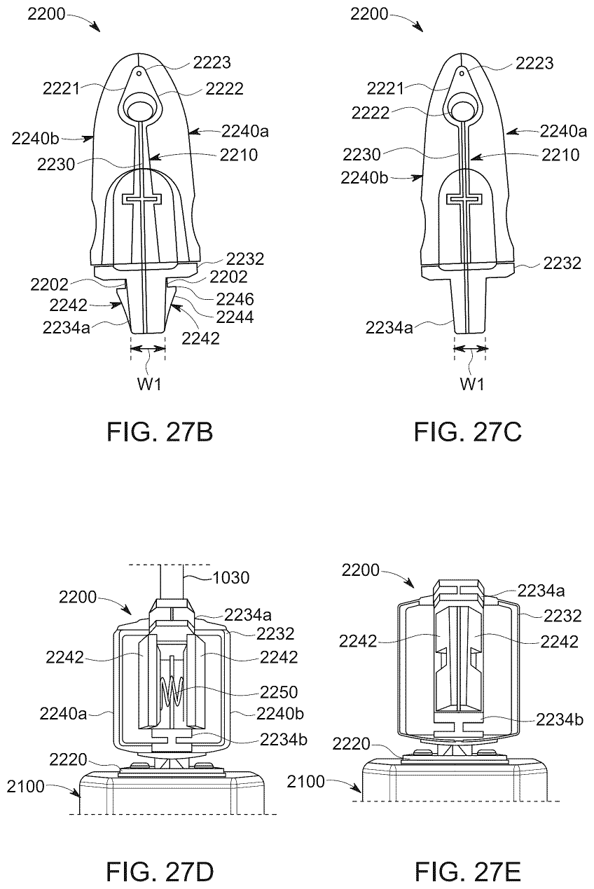

FIGS. 27A-27E depict one example of proximal attachment assembly 2200. Proximal attachment assembly 2200 may be positioned between inner guide catheter handle 2100 and delivery system handle 1010. Proximal attachment assembly 2200 generally includes one or more of a support frame 2210 and first and second engagement members 2240a, 2240b moveably attached to support frame 2210.

In the embodiment depicted, support frame or support body 2210 may include an upper portion and a lower portion extending from the upper portion. The upper portion may include a mount member 2220 which may be configured to mount or otherwise connect to inner guide catheter handle 2100, as shown in the example of FIGS. 27D and 27E. In the embodiment depicted, mount member 2220 may be in the form of a mounting plate which may be secured to inner guide catheter 2100 via one or more fasteners. However, in other embodiments, mount member 2220 may be connected to inner guide catheter handle 2100 via welding, for example, or mount member 2220 may be integral with inner guide catheter handle 2100 such as via an injection molding process, as another example.

The upper portion may, for example, also include a boss 2221 extending proximally from mount member 2220. Boss 2221 may define a first opening 2222 and a second opening 2223 which may each extend at least partially into boss 2221 and in a proximal-distal direction. First opening 2222 may be configured to receive brake shaft 1030 such that brake shaft 1030 may pass through proximal attachment assembly 2200 as it extends from delivery system handle 1010 to inner guide catheter handle 2100 where it is fixedly secured. However, in some embodiments, proximal attachment assembly 2200 may fixedly secure brake shaft 1030 to inner guide catheter handle 2100. Second opening or pin opening 2223 may be offset in an upward direction from first opening 2222 and may be generally parallel relative thereto. Second opening 2223 may be configured to receive a hinge pin 2204, as shown in the example of FIG. 27A.

The lower portion of proximal attachment assembly 2220 may include a central support wall or central member 2230, a transverse plate or transverse member 2232, and first and second fingers 2234a, 2234b. As shown in the example of FIGS. 27B and 27C, central support wall 2230 may extend downwardly from boss 2221 and may extend in a proximal-distal direction. Transverse plate 2232 may extend transverse to central support wall 2230, such as perpendicular (±5 degrees) relative thereto, and may be connected to a lower end of central support wall 2230 opposite an upper end thereof which may be connected to boss 2221, according to one example of the disclosure. First finger or first end wall 2234a and second finger or second end wall 2234b may extend from transverse plate 2232 in a downward direction and may be respectively disposed at proximal and distal ends of transverse plate 2232 so as to form a gap therebetween, for example. As shown in FIGS. 27B and 27C, fingers 2234a, 2234b each define a width W1 which extends in a direction transverse to the proximal-distal direction.

First and second engagement members 2240a, 2240b may be pivotably connected to support frame 2210 in one example. In this regard, first and second members 2240a, 2240b may, for example, be connected to each other via hinge pin 2204 which extends through boss 2221 of proximal attachment assembly, as mentioned above. Such pivotable connection may be made at or near respective first ends of engagement members 2240a, 2240b. As shown in the illustrated example, first and second engagement members 2240a, 2240b extend about boss 2221 and downwardly at opposite sides of central support wall 2230. Boss 2221 may have a tear-drop shaped profile which may correspond to a tear-drop shape opening defined by first and second engagement members 2240a, 2240b. This shape may facilitate pivotable action of first and second engagement members 2240a, 2240b about boss 2221. A spring 2250 may be positioned between first and second engagement members 2240a, 2240b such that the spring 2250 biases engagement members 2240a, 2240b toward a first position or locked configuration away from each other, as shown in FIG. 27D. In other words, when first and second engagement members 2240a, 2240b are in the first position, they may be in an expanded state. However, pressing on engagement members 2240a, 2240b against the bias of spring 2250 may move engagement members 2240a, 2240b to a second position or unlocked configuration in which they may abut each other, as shown in the example of FIG. 27E. Thus, when first and second members 2240a, 2240b are in the second position, they may be in a collapsed state according to one example of the disclosure.

Each engagement member 2240a, 2240b may, in various examples, also include a hook member or capture member 2242 extending downwardly from a second end thereof. Such capture members 2242 may each extend through transverse plate 2232 of support frame 2210 and within the gap between fingers 2234a, 2234b of support frame 2210. Thus, in one example of the first position, first and second engagement members 2240a, 2240b may engage transverse plate 2232 to limit or stop their outward expansion. Capture members 2242 may each include a sloping surface 2244 that slopes outwardly and terminates at a shoulder 2246 which faces transverse plate 2232, as best shown in the example of FIG. 27B. Each shoulder 2246 and transverse plate 2232 may define a slot or channel 2202 when first and second engagement members 2240a, 2240b are in the first position. Such slot 2202 may be configured to slidably receive a corresponding rail of stabilizer 4000, which is described in more detail below. Thus, in the example first position, capture members 2242 may extend beyond width W1 of fingers 2234a, 2234b. In other words, capture members 2242 in the first position can, in one example, collectively define a width greater than width W1 of fingers 2234a, 2234b. However, according to one example, when engagement members 2240a, 2240b are in the second position, as shown in FIG. 27C, capture members 2242 are either flush with or narrower than the width W1 of fingers 2234a, 2234b. Although it has been described that first and second engagement members 2240a, 2240b are each moveable relative to support frame 2210, it should be understood that in some embodiments one of such engagement members 2240a, 2240b may be static, while the other engagement member 2240a, 2240b may be dynamic. In other words, one of engagement members 2240a, 2240b may only be moveable relative to support frame 2210 between the first and second configurations (i.e., locked and unlocked configurations).

FIGS. 29A and 29B depict one example of distal attachment assembly 3200. Distal attachment assembly 3200 can be positioned distal of outer guide catheter handle 3100. Thus, in the embodiment depicted in FIG. 20, inner and outer guide catheter handles 2100, 3100 are positioned between proximal and distal attachments 2200, 3200. Distal attachment assembly 3200 may also have a snap-fit mechanism or the like. However, the snap-fit mechanism of distal attachment may be configured to constrain all movement when attached to stabilizer 4000. Distal attachment 3200 may generally include one or more of a bushing 3210, a housing 3220, and a connection mechanism for connecting to stabilizer 4000.

Bushing or shaft 3210 may define a central opening 3212 through which outer guide catheter 3300 may extend, as shown in the example of FIG. 29A. Bushing 3210 may extend into outer guide catheter handle 3100 and be connected to a distal end of outer guide catheter handle 3100 via a collar 3112 which may have a set screw that rotationally and translationally secures bushing 3210 according to various examples.

Housing or support body 3220 may, in one example, surround the portion of bushing 3210 extending from outer guide catheter handle 3100 and may include a lower housing portion 3220a and an upper housing portion 3220b. As shown in the example of FIG. 29B, one or more O-rings 3240 may be positioned between bushing 3210 and housing 3220. For example, distal attachment 3200 may have four O-rings 3240 with two O-rings 3240 located at a proximal location and two O-rings 3240 located at a distal position. This distribution of O-rings 3240 helps to ensure even distribution of load/compression. However, other configurations are contemplated, such as two or more O-rings 3240 distributed at even intervals along a length of bushing 3210. To help contain O-rings 3240, lower housing portion 3220a and bushing 3210 may have corresponding lips 3215, 3225 preventing proximal-distal travel of O-rings 3240. O-rings 3240 may be made from a material that enhances friction, such as silicon, for example. This arrangement helps provide rotational friction between housing 3220 and bushing 3210 and prevent unintentional rotation of outer and inner guide catheters 2300, 3300 while utilizing inner and outer guide catheter handles 2100, 3100. In other words, O-rings 3240 help provide constant friction to prevent unintentional rotation of inner and outer catheters 2300, 3300. This O-ring configuration also helps coaxially correct inner and outer guide catheters 2300, 3300 to help keep them coaxially aligned. Furthermore, O-rings 3240 help protect outer catheter 3300 by removing direct contact of catheter 3300 and by promoting handle torque transmission directly to O-rings 3240 rather than directly from handle 3100 to catheter 3300 in order to brake the system.

Lower housing portion 3220a may include a connection mechanism for connecting distal attachment 3200 to stabilizer 4000, for example. In the depicted embodiment, the connection mechanism may include a dynamic pin or first engagement member 3226a and a static pin or second engagement member 3226b, for example. Static pin 3226b may extend through a first opening 3224 in lower housing 3220a, while dynamic pin 3226a may extend through a second opening 3222 in lower housing 3220a, as best shown in the example of FIG. 29B. In one example, second opening 3222 may be an elongate opening or elongate slot allowing pin 3226a to dynamically move in a proximal-distal direction. Lower housing 3220a may include a chamber 3228 with a spring or other biasing element 3250 disposed therein, for example. Spring 3250 may bias dynamic pin 3226a in a first position or proximal position. When the spring bias is overcome, dynamic pin 3226a may move to a second position or second position which is closer to static pin 3226b than the first position. Thus, pins 3226a, 3226b have a first configuration when pin 3226a is in the first position and a second configuration 3226b when pin 3226a is in the second position, according to an example of the disclosure.

FIGS. 30A-30C depict stabilizer 4000 according to an embodiment of the present disclosure. Stabilizer 4000 may generally include one or more of a base 4010, one or more support arms 4020, a distal attachment 4030, and a proximal attachment 4040.

Base or platform 4010 may be a plate with a flat bottom for placement on a flat surface of a table or the like. Base 4010 may define a longitudinal axis A3 and may include one or more handles 4012 for manipulating stabilizer 400. For example, as shown in FIG. 30A, base 4010 may include one or more of handles 4012 at proximal and distal ends of base 4010.

Support arm 4020 may extend upwardly from base 4010 and may support both distal attachment 4030 and proximal attachment 4040, as shown in the example of FIG. 30A. However, in other embodiments, separate support arms 4020 may extend from base 4010 that may separately support distal and proximal attachments 4030, 4040.

Proximal attachment or first attachment 4040 may include one or more of a first rail or first member 4042a, a second rail or second member 4042b, a first end wall 4044a, and a second end wall 4044b. First and second rails 4042a, 4042b may extend parallel (±5 degrees) to each other between first and second end walls 4044a, 4044b and may each have a planar upper surface 4043 and planar lower surface (not shown) according to one example of the disclosure. End walls 4044a, 4044b and rails 4042a, 4042b may define an elongate slot 4046 that may be rectangular in shape, as shown in the example of FIG. 30C. However, elongate slot 4046 may also have other shapes, such as oval or pill-shaped, for example. Elongate slot 4046 may have a width slightly wider than width W1 of fingers 2234a, 2234b of proximal attachment 2220. Additionally, elongate slot 4046 may have a length L2 which may be defined from first end wall 4044a to second end wall 4044b. As explained in more detail below, this length L2 may correspond to a distance that inner guide catheter 2300 may translate relative to outer guide catheter 3300. In this regard, first and second end walls 4044a, 4044b form stop surfaces for proximal attachment assembly 2200 and correspondingly a translational limit for inner guide catheter handle 2100. Proximal attachment 2200 may also include proximal and distal tabs 4048a, 4048b extending downward therefrom, for example. Such tabs 4048a, 4048b may be used as leverage points for manual manipulation and translation of inner guide catheter handle 2100 when proximal attachment assembly 2200 is connected to proximal attachment 4040. In one example, proximal attachment 4040 extends along a longitudinal axis A4 which may form an acute angle relative to axis A3 of base 4010.

Distal attachment or second attachment 4030 may include a pair of attachment members 4032 and a recess 4031 extending therebetween. As shown in the example of FIG. 30B, each attachment member 4032 may include a first hook or first capture member 4034a and a second hook or second capture member 4034b. First capture member 4034a extends in a distal direction while second capture member 4034b extends in a proximal direction such that each capture member 4034a, 4034b defines a corresponding recess 4035a, 4035b for receipt of dynamic pin 3226a and static pin 3226b, respectively. Additionally, first capture member 4034a may include a cam surface 4038 that slopes in a proximal to distal direction. Such cam surface 4038 may be configured to deflect dynamic pin 3226a of distal attachment assembly 3200 from its first position to its second position thereby allowing dynamic pin 3226a to pass by first hook 4034a and into its corresponding recess 4035a where it returns to the first position or somewhere between the first and second positions. Each attachment member 4032 may also include an intermediate projection 4036 that defines sloped surface 4037 that is sloped toward second hook 4034b in various examples. This sloped surface 4037 may direct static pin 3226b of distal attachment assembly 3200 into recess 4035b. Attachment members 4032 may be laterally offset from each other and may extend parallel relative to each other (±5 degrees) so as to define recess 4031 which may be configured to receive lower housing portion 3220a of distal attachment assembly 3200 according to various examples of the disclosure.

Stabilizer 4000 may be made from a single sheet of metal material such that at least the base 4010, support arm 4020, and proximal attachment 4040 are stamped from the single sheet of metal material and bent into their depicted configuration according to one example of the disclosure. The single-sheet construction provides several advantages over multi-component assemblies, including enhanced structural integrity, reduced manufacturing complexity, and elimination of potential failure points at component junctions. The metal material may include, but is not limited to, stainless steel, titanium alloys, aluminum alloys, and the like. Distal attachment 4030 may be separately manufactured and connected to support arm 4020, such as by welding, for example. However, in other embodiments distal attachment 4030 may also be stamped out of the single sheet of metal material. The depicted configuration of stabilizer 4000 may reduce welding points, which may be potential points of failure, and may reduce cleaning and sterilization risks.

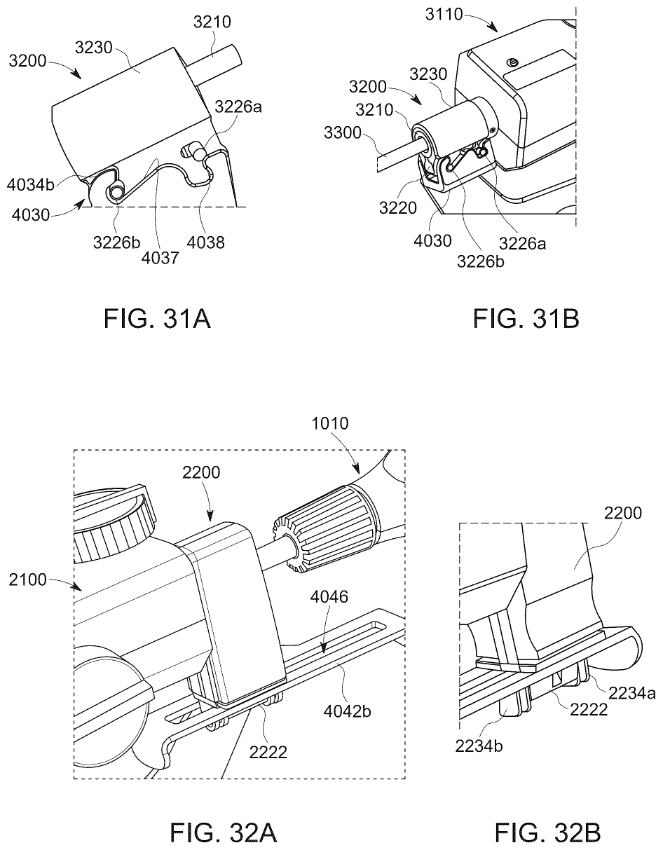

FIGS. 31A and 31B illustrate one example of the connection between distal attachment 4030 of stabilizer 4000 and distal attachment assembly 3200 of steerable guide system 5. In this regard, distal attachment assembly 3200 may be attached to distal attachment 4030 first before proximal attachment assembly 2200. This may be achieved by sliding static pin 3226b along sloped surface 4037 of intermediate projection 4036 and in the distal direction until pin 3226b is fully received within recess 4035b and captured by first hook 4034b, as illustrated in the example of FIG. 31A. At this point, distal attachment assembly 3200 may be rotated about first pin 3226b in a downward direction until dynamic pin 3226a contacts cam surface 4038. Further downward rotation causes cam surface 4038 to urge dynamic pin 3226a from its first position to its second position which clears first hook 4034a allowing dynamic pin 3226a to be received within recess 4035a. Once dynamic pin 3226a is received within recess 4035a, it returns to the first position or somewhere between the first and second positions thereby locking distal attachment assembly 3200 to distal attachment 4030 of stabilizer 4000, as illustrated in the example of FIG. 31B.

FIGS. 32A and 32B illustrate one example of the connection between proximal attachment 4040 of stabilizer 4000 and proximal attachment assembly 2200 of steerable guide system 5. In this regard, once distal attachment assembly 3200 is secured to distal attachment 4030, proximal attachment assembly 2200 may be lowered toward elongate slot 4046 of proximal attachment 4040. As proximal attachment assembly 2200 is lowered, fingers 2234a, 2234b and capture members 2242 are received within elongate slot 4046 such that rails 4042a, 4042b engage sloped surfaces 2244 of capture members 2242 and urge them inwardly from their first position to their second position in which their collective width is equal to or lesser than width of fingers 2234a, 2234b. However, since width W1 of fingers 2234a, 2234b is slightly less than that of elongate slot 4046, fingers 2234a, 2234b may advance into elongate slot 4046. Once rails 4042a, 4042b pass shoulders 2046, the bias of spring 2250 returns hook capture 2242 and corresponding engagement members 2240a, 2240b to their respective first position. In this configuration, first and second rails 4042a, 4042b are received in their corresponding slots 2022. Additionally, spring 2250 pushes capture members 2242 outwardly into contact with rails 4042a, 4042b with sufficient force so as to create friction between them which limits inadvertent movement of proximal attachment assembly 2220 along elongate slot 4046.

Although it has been described that proximal attachment assembly 2200 of the multi-catheter system includes moveable engagement members 2240a, 2240b that are configured to attach to rails 4042a, 4042b and translate within slot 4046 of proximal attachment 4040, it should be understood that this arrangement can be reversed. For example, stabilizer 4000 may have a proximal attachment with engagement members, like engagement members 2240a, 2240b, that are moveable between first and second positions, and the multi-catheter system may include a proximal attachment with rails and an elongate slot, like rails 4042a, 4042b and slot 40446, for receipt of such engagement members of the stabilizer. Similarly, the configurations of distal attachment assembly 3200 of the multi-catheter system and distal attachment 4030 can be reversed. Thus, stabilizer 4000 may have a distal attachment assembly with a static and dynamic pin, such as static and dynamic pins 3226a, 3226b, and the multi-catheter system may have a distal attachment with attachment members, like attachment members 4032 for receiving and engaging with the static and dynamic pins.

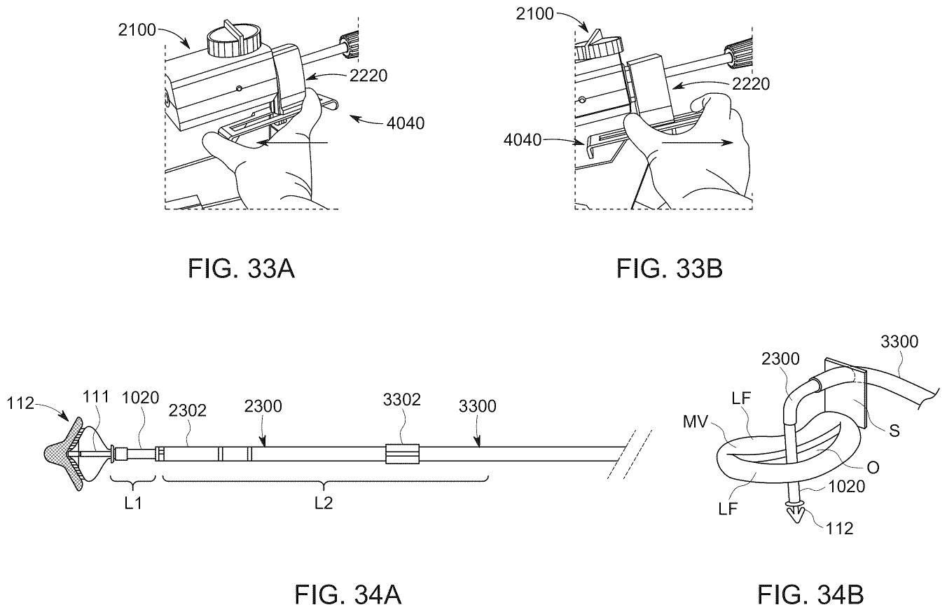

FIGS. 33A-34B illustrate the use of interventional system 5 to position fixation device 112 within a target valve, which is depicted as a mitral valve MV, but should be understood to alternatively be a tricuspid valve TV (see FIG. 1A). In a transcatheter procedure for repairing mitral valve MV, distal end 3302 of outer guide catheter 3300 is advanced through the interatrial septum S and positioned within the right atrium proximal to mitral valve MV. To help position fixation device 112 above the desired location along the line of coaptation and within the valve orifice O, inner guide catheter 2300 may be advanced from outer guide catheter 3300. This may be achieved by sliding proximal attachment assembly 2100 distally (i.e., forward) within elongate slot 4046 of proximal attachment 4040, as shown in FIG. 33A. The practitioner may utilize distal tab 4048b as a leverage point to advance proximal attachment assembly 2100. Should it be determined that it was advanced too far, the practitioner may slide proximal attachment assembly 2100 proximally (i.e., backward) optionally utilizing proximal tab 4048a as a leverage point, as shown in FIG. 33B. As illustrated in FIG. 34A, the maximum length at which inner guide catheter 2300 can advance from outer guide catheter is length L2, which corresponds to the length of elongate slot 4046. This length L2 may be determined via a statistical analysis of heart geometries of a select patient population. For example, the dimensions of the left atria of the select population may be analyzed up to the first, second, or third standard deviation such that when inner guide catheter 2300 is advanced to the maximum length L2 from outer guide catheter 3300, fixation device 112 does not bump into and damage any structures of the atrium. Thus, elongate slot 4046 of proximal attachment 4040 forms a “parking spot” or “landing zone” in which, when proximal attachment assembly 2100 is as proximal as possible (i.e., abutting first end wall 4044a), fixation device 112 is positioned within and maintained within distal end 3302 of outer guide catheter 3300. This provides the operator with visual confirmation that fixation device 112 is sheathed so that fluoroscopic confirmation is not needed which helps reduce the overall time the patient and operator are exposed to fluoroscopy and enhances patient safety. As shown in FIG. 34B, inner guide catheter 2300 may also be curved so that fixation device 112 may be oriented toward mitral valve.

Once fixation device 112 is positioned above the desired location along the line of coaptation, delivery catheter 1020 may be advanced from inner guide catheter 2300 into the valve. This may be achieved by unlocking fastening assembly 1500 by rotating fastening knob 1520 in the first rotation direction R1, according to one example. Delivery system handle 1010 and delivery catheter 1020 may then be advanced along brake shaft 1030 until the desired position within valve MV is achieved, at which point fastening assembly 1500 may be locked by rotating knob 1520 in the second rotation direction R2. The maximum length at which delivery catheter 1020 can advance from inner guide catheter is length L1, which corresponds to the maximum distance handle 1010 can advance along brake shaft 1020. Again, this length can be determined from a population level analysis of heart geometries to set the L1 limit. From there, leaflets LF may be grasped. Once the leaflets LF have been sufficiently grasped and fixation device 112 is moved to the closed position, lock line 102 may be released, as described above. Additionally, deployment system 1170 may be disengaged from slider 1110 and actuator shaft 1140. Actuator shaft 1140 may then be rotated to release actuator rod 170 from stud 131 or base 139 and retracted which releases coupling member 160 from shaft 111 and also releases proximal element lines 101a, 101b from shaft 111, as also described above.

Although interventional system 3 has been described as including fixation device 112, delivery system 1000, steerable guide system 5, and stabilizer 4000, it should be understood that interventional system 3 can include more, less, or a different arrangement of components. For example, interventional system 3 may not include stabilizer 4000, which may be appropriate for procedures where manual stabilization is preferred or where limited procedure space precludes the use of a separate stabilizing apparatus. In another example of interventional system 3, steerable guide system 5 may be integrated into delivery system 1000 such that delivery catheter 1020 may itself be steerable via controls integrated into delivery handle 1010. This integrated approach could reduce the overall system profile and simplify the procedural workflow by eliminating separate catheter systems. In an even further example of interventional system 3, steerable guide system 5 may alternatively be configured such that a single guide catheter provides all the steering inputs to guide fixation device 112 to the target valve rather than inner and outer guide catheter assemblies 2000, 3000 for performing the same. This single-catheter configuration might be advantageous for specific anatomical approaches or in pediatric applications where space constraints are significant. In a still further embodiment, a robotically guided catheter (not shown) may be provided in interventional system 3 to both stabilize and steer delivery system 1000 and fixation device 112 to the target valve. The robotic system could incorporate haptic feedback mechanisms to provide the operator with tactile sensation during the procedure while offering enhanced stability and precision. However, in such robotically assisted procedure, a practitioner may still manually grasp leaflets and deploy fixation device 112 via the controls of delivery device system 1000.

Although the subject matter disclosed herein has been described with reference to particular embodiments, it is to be understood that these embodiments are merely illustrative of the principles and applications set forth in this disclosure. It is therefore to be understood that numerous modifications may be made to the exemplary embodiments and that other arrangements may be devised, such as combining one or more features of one embodiment with another embodiment or features from a plurality of embodiments, as an example. Thus, the exemplary embodiments herein are not intended to be exhaustive or to limit the disclosed subject matter to such embodiments.

1. A delivery system for delivering a fixation device to a heart valve, comprising:
a delivery catheter having a proximal end portion and a distal end portion, the delivery catheter being configured to releasably couple to the fixation device;
a delivery handle coupled to the proximal end portion of the delivery catheter and having a lock control assembly, the lock control assembly having a lock line handle releasably coupled to the delivery handle and actuatable between a locked position and an unlocked position, the lock line handle having a handle shaft, the handle shaft having a shaft body defining a first opening and a lever arm defining a second opening, the lever arm being connected to the shaft body and being rotatable relative to the shaft body between a first position in which the first and second openings are axially aligned and a second position in which the first and second openings are angled relative to each other; and
a lock line for locking and unlocking the fixation device having a first end portion fixedly coupled to the lock line handle and a second end portion extending into the first and second openings of the lock line handle;
wherein, when the lever arm is in the first position, the second end portion of the lock line is free to travel through the first and second openings, and when the lever arm is in the second position, the second end portion of the lock line is secured to the handle shaft such that it is prevented from moving through the first and second openings.

2. The system of claim 1, wherein:
the lever arm includes a lever body and a post extending from the lever body, the post defining the second opening through which the lock line extends, and
the shaft body defines a post opening configured to receive the post of the lever arm and the first opening intersects the post opening.

3. The system of claim 2, wherein the lever body includes a third opening, the third opening being axially aligned with the second opening such that the second end portion of the lock line extends into the first, second, and third openings.

4. The system of claim 1, wherein:
the shaft body includes a post extending from the shaft body, the post defining the first opening through which the lock line extends, and
the lever arm defines a post opening configured to receive the post of the shaft body and the second opening intersects the post opening.

5. The system of claim 1, wherein the lock line handle includes a lock knob and the handle shaft extends from the lock knob, the lock knob having an opening extending therethrough, and the handle shaft extends through the opening of the lock knob such that the lever arm is positioned at one side of the lock knob and the shaft body is at least partially positioned at another side of the lock knob.

6. The system of claim 5, wherein the lock line handle includes a visual indicator that visually indicates when the lock line handle is in the locked position or the unlocked position.

7. The system of claim 6, wherein the visual indicator comprises the lock knob which has a pear shape that acts as a direction-indicating pointer, the lock knob changing orientation between a first position where the pointer indicates a distal direction corresponding to the locked position and a second position where the pointer indicates an upward direction corresponding to the unlocked position.

8. The system of claim 5, wherein the lock line handle includes a cylindrical body extending from the lock knob and at least partially about the handle shaft, the cylindrical body having a plurality of detents extending into an exterior thereof.

9. The system of claim 8, wherein the lock control assembly includes a locking system having a lock body coupled to the delivery handle, the lock body having a pin configured to independently engage the detents in the cylindrical body, wherein the detents include at least a first detent representing an unlocked position in which the lock line is tensioned to unlock the fixation device, and a second detent representing a locked position in which tension on the lock line is released to lock the fixation device, wherein the pin and detents together provide tactile feedback to a user regarding the tension applied to the lock line when the lock handle is rotated between positions.

10. The system of claim 9, wherein the cylindrical body includes a circumferential channel intersecting the plurality of detents, the pin is configured to engage the circumferential channel when not engaged with any of the detents, and the lock body is moveable between a locked position in which the pin engages one of the channel and the detents and an unlocked position in which the pin is disengaged from the channel and the detents which permits the lock line handle from being released from the delivery handle.

11. The system of claim 10, wherein the lock control assembly includes a lock handle housing, the lock handle housing having a lock handle extension configured to receive the cylindrical body of the lock knob assembly, and the lock body is coupled to the lock handle extension.

12. The system of claim 11, wherein the locking system includes a biasing member positioned between the lock body and the lock handle extension, the biasing member urging the lock body toward the locked position thereof.

13. The system of claim 11, wherein:
the lock handle housing includes a fluid volume and an inlet/outlet port in fluid communication with the fluid volume, and
the lock control assembly further includes a spool, the spool being received within the fluid volume of the lock handle housing and having a spool track along which the first end portion of the lock line extends, and wherein the spool is releasably coupled to the lock handle shaft and is configured to rotate with the lock line handle such that rotating in a second direction releases tension on the lock line.

14. The system of claim 13, wherein:
the spool includes a first lock line opening and a second lock line opening,
the first end portion of the lock line extends through the first lock line opening of the spool and into the first opening of the shaft body of the lock handle shaft where the first end portion of the lock line is fixedly secured, and
the second end portion of the lock line extends through the second lock line opening in the spool, the first opening in the shaft body, and the second opening in the lever arm.

15. The system of claim 13, wherein the delivery handle includes an actuator rod for moving the fixation device between a plurality of positions, the actuator rod extending through the lock handle housing and into the delivery catheter.

16. An interventional system for repairing a heart valve, comprising:
a multi-catheter system comprising:
a plurality of catheters,
a plurality of catheter handles coupled to respective ones of the plurality of catheters,
a first attachment assembly having a support body coupled to one of the catheter handles and first and second engagement members connected to the support body, at least one of the first and second members being moveable relative to the support body such that the first and second engagement members have a first configuration and a second configuration, wherein the first and second engagement members are further apart in the first configuration than in the second configuration, and
a second attachment assembly; and
a stabilizer for supporting the multi-catheter system having a base, a first attachment coupled to the base, and a second attachment coupled to the base, the first attachment being configured to attach to the first attachment assembly and having a first rail and a second rail, the first and second rails at least partially defining a slot disposed therebetween, and the second attachment being configured to attach to the second attachment,
wherein, the first and second engagement members are receivable within the slot while in the second configuration and engage the rails when in the second configuration so as to retain the first attachment assembly within the slot.

17. The system of claim 16, wherein the first attachment assembly is slidable along the first and second rails when the first and second engagement members are disposed within the slot and in the first configuration.

18. The system of claim 16, wherein the first attachment assembly includes a biasing member, the biasing member urging the first and second engagement members toward the first configuration.

19. The system of claim 16, wherein the first and second engagement members each include a capture member, the capture member of the first engagement member having a shoulder at least partially defining a first channel, and the capture member of the second engagement member having a shoulder at least partially defining a second channel, the first and second channels being configured to respectively receive the first and second rails when the first and second engagement members are in the first configuration.

20. An interventional system for repairing a heart valve, comprising:
a multi-catheter system comprising:
a plurality of catheters,
a plurality of catheter handles coupled to respective ones of the plurality of catheters,
a first attachment assembly, and
a second attachment assembly having a support body coupled to one of the catheter handles and first and second engagement members connected to the support body, the first engagement member being moveable relative to the second engagement member between a first position and a second position; and
a stabilizer for supporting the multi-catheter system having a base, a first attachment coupled to the base, and a second attachment coupled to the base, the first attachment being configured to couple to the first attachment assembly, and the second attachment having a first capture member defining a first recess configured to receive the first engagement member and a second capture member defining a second recess configured to receive the second engagement member,
wherein, when the second engagement member is received within the second recess, moving the first engagement member from the first position to the second position allows it to be received within the first recess.
